(19) United States
(12) Patent Application Publication
(10) Pub. No.: US 2022/0304950 A1
(43) Pub. Date: Sep. 29, 2022

(54) ESKETAMINE FOR THE TREATMENT OF PATIENTS WITH MAJOR DEPRESSIVE DISORDER, INCLUDING SUICIDALITY

(71) Applicant: JANSSEN PHARMACEUTICALS, INC., Titusville, NJ (US)

(72) Inventors: Carla M. CANUSO, Titusville, NJ (US); Dong-Jing FU, Titusville, NJ (US); Dawn F. IONESCU, Titusville, NJ (US); Roseanne LANE, Titusville, NJ (US)

(21) Appl. No.: 17/638,388

(22) PCT Filed: Aug. 28, 2020
(86) PCT No.: PCT/IB2020/058030

§ 371 Date: Feb. 25, 2022

Related U.S. Application Data
(60) Provisional application No. 62/892,841, filed on Aug. 28, 2019.
(60) Provisional application No. 62/897,593, filed on Sep. 9, 2019.

(51) Int. Cl. A61K 31/135; A61K 9/00; A61K 9/08; A61P 25/24; A61K 45/06

(52) U.S. Cl. CPC A61K 31/135; A61K 9/0043; A61K 9/08; A61P 25/24; A61K 45/06

(57) ABSTRACT

The disclosure relates to methods for reducing symptoms of major depressive disorder, including suicidality, in a human patient assessed to be at imminent risk for suicide comprising the administration of esketamine in addition to standard of care treatment. In certain embodiments, the methods comprise determining if the patient has previously attempted suicide, and, if so, treating such patient with a standard of care treatment and a therapeutically effective amount of esketamine, whereas patients determined not to have previously attempted suicide are administered standard of care treatment without treating the patient with esketamine.

### CROSS-REFERENCE TO RELATED APPLICATIONS

This application claims the benefit of U.S. Provisional Patent Application No. 62/892,841, filed Aug. 28, 2019, and U.S. Provisional Patent Application No. 62/897,593, filed Sep. 9, 2019, the disclosures of which are incorporated herein by reference in their entireties.

### TECHNICAL FIELD

The present invention relates to treatments for reducing the symptoms of major depressive disorder (MDD), including suicidality, in patients assessed to be at imminent risk for suicide.

### BACKGROUND

Suicide is among the leading causes of death worldwide. MDD is the condition most frequently associated with suicide. Patients with MDD presenting with active suicidal ideation with intent are at imminent risk for suicide and constitute a psychiatric emergency that requires immediate intervention. There are, however, no approved treatments for the rapid reduction of the symptoms of MDD with suicidal ideation, and these patients are typically hospitalized.

While currently available antidepressants are effective in treating depressive symptomatology, their onset of effect takes 3 to 6 weeks. This delay is potentially dangerous, especially since suicide risk is highest early in treatment. Indeed, the incidence of attempted suicide in adult patients with MDD ranges between 10% to 20%, and the reported prevalence of suicidal ideation is as high as 60% in this population. Moreover, those treated for depression as inpatients following suicide ideation or suicide attempts are about three times as likely to die by suicide (6%) as those who were only treated as outpatients.

Further, patients with significant suicidal ideation and behavior are typically excluded from antidepressant treatment trials. Given the seriousness of this disease and the great unmet clinical need for efficacious treatments, improved methods for treating patients assessed to be an imminent risk for suicide are needed.

### SUMMARY

In some embodiments, the disclosure relates to methods for reducing symptoms of major depressive disorder, including suicidality, in a human patient assessed to be at imminent risk for suicide comprising the administration of esketamine in addition to standard of care treatment. In certain embodiments, the methods comprise determining if the patient has previously attempted suicide, and, if so, treating such patient with a standard of care treatment and a therapeutically effective amount of esketamine. In other aspects, to the extent a patient is determined not to have previously attempted suicide, the patient is administered standard of care treatment without treating the patient with esketamine.

### BRIEF DESCRIPTION OF THE DRAWINGS

FIG. 1 is the study design for Example 1.

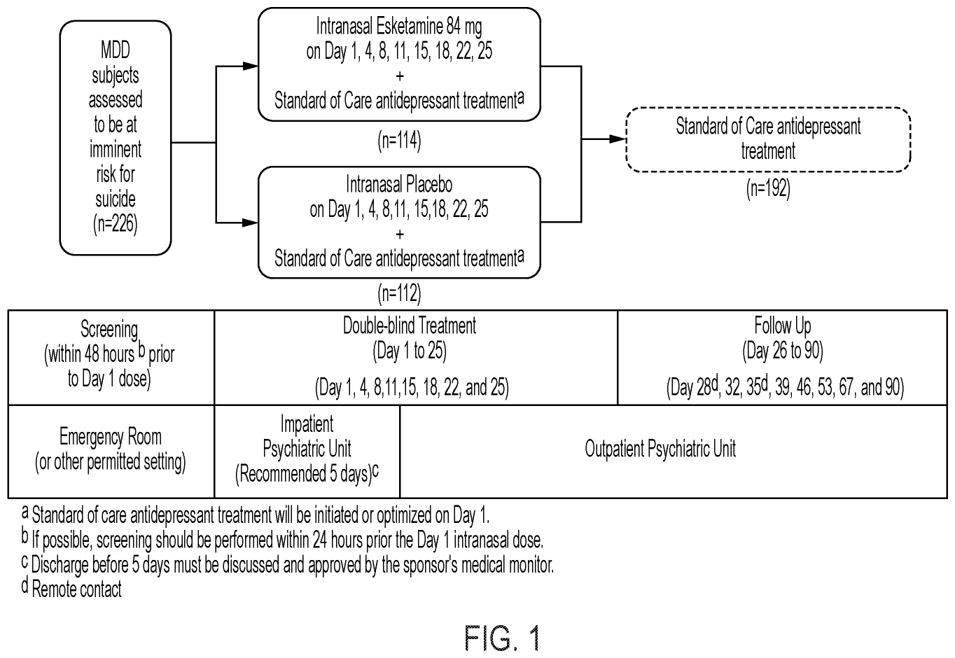

FIG. 2 is a line graph showing Montgomery-Asberg Depression Rating Scale (MADRS) Total Score: LS Mean (+/−SE) of Changes Over Time-ANCOVA LOCF; Double-blind Treatment Phase for the full efficacy analysis set. The LS mean and SE were based on analysis of covariance (ANCOVA) model with treatment (placebo, esketamine 84 mg), analysis center, standard of care antidepressant treatment as randomized (antidepressant monotherapy, antidepressant plus augmentation therapy) as factors and baseline value as a covariate. A negative change in score indicates improvement.

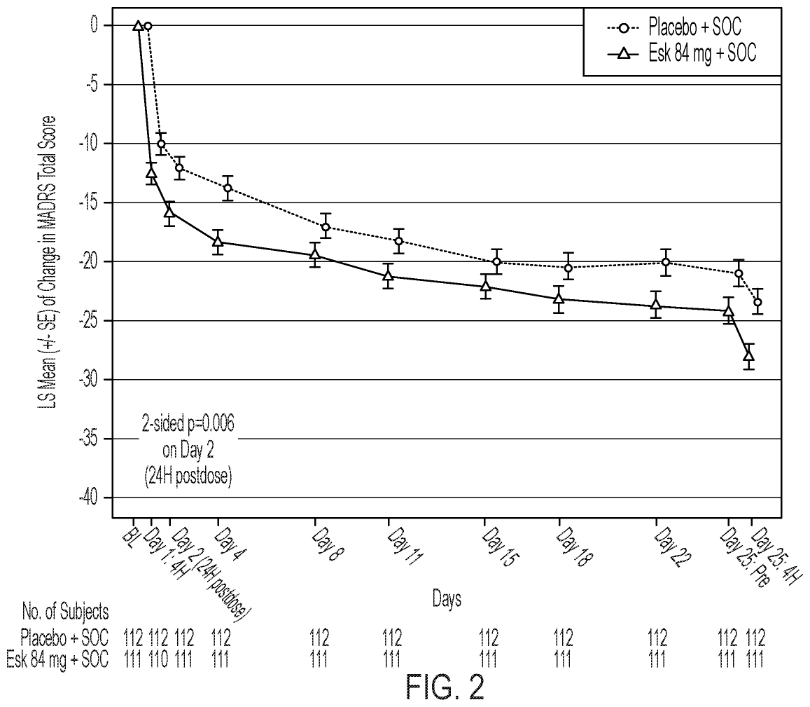

FIG. 3 is a bar graph showing the Clinical Global Impression-Severity of Suicidality-Revised (CGI-SS-R) Score: Frequency Distribution at Baseline, 4 Hours Post First Dose, 24 Hours Post First Dose and Day 25; LOCF; Double-blind Treatment Phase for the full efficacy analysis set.

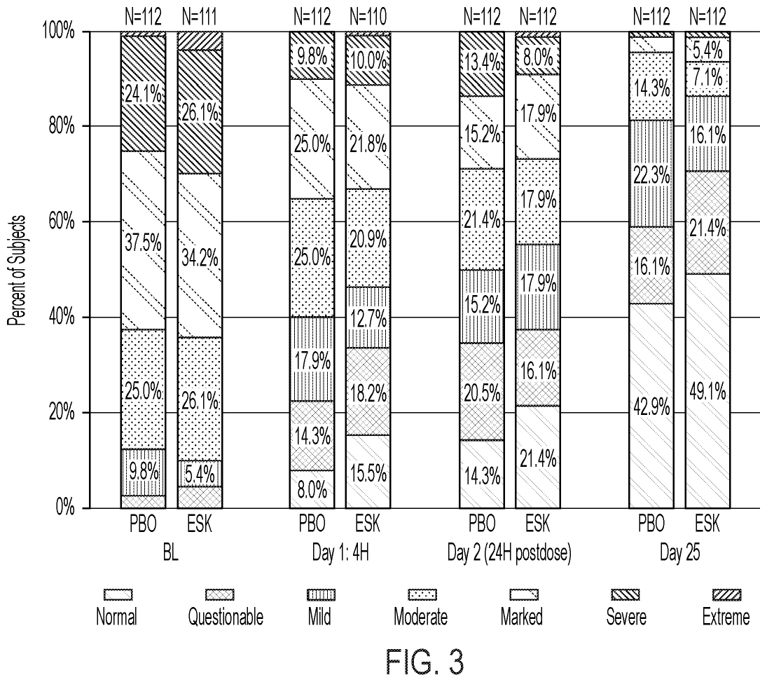

FIG. 4 is a line graph showing the Montgomery-Asberg Depression Rating Scale (MADRS) Total Score: LS Mean (+/−SE) of Changes Over Time-MMRM Observed Case; Double-blind Treatment Phase for the full efficacy analysis set. The LS mean and SE were based on MMRM analysis with treatment (placebo, esketamine 84 mg), time, analysis center, standard of care antidepressant treatment as randomized (antidepressant monotherapy, antidepressant plus augmentation therapy), time by treatment interaction as factors and baseline value as a covariate. A negative change in score indicates improvement.

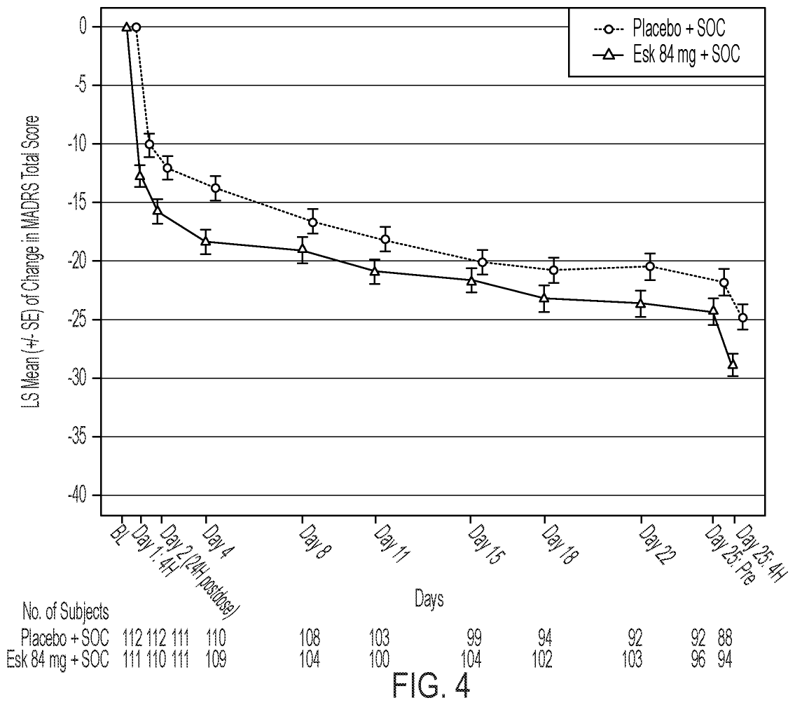

FIG. 5 is the study design for Example 2.

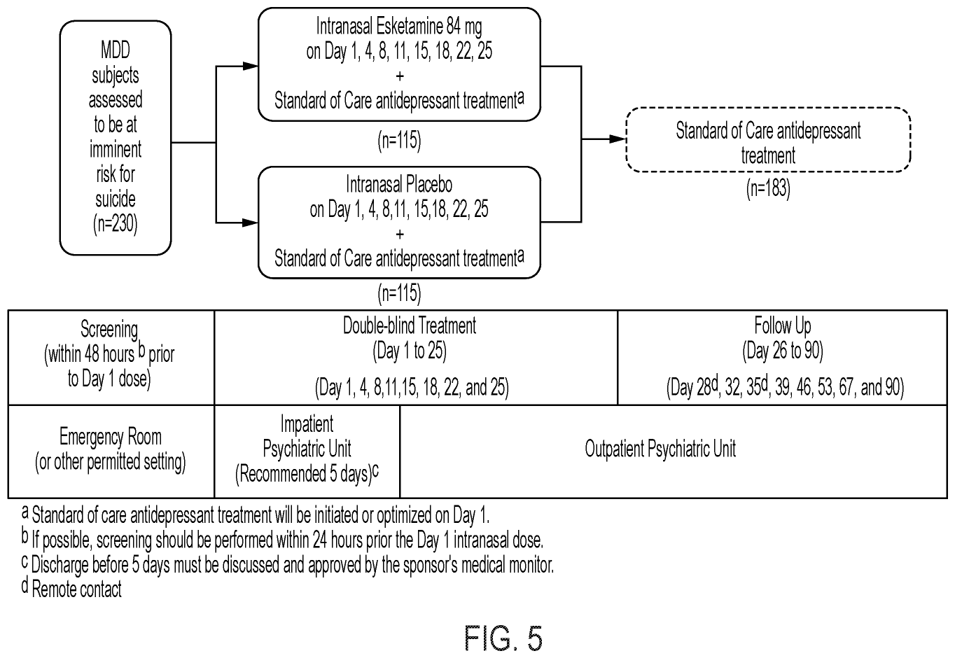

FIG. 6 is a line graph showing the Montgomery-Asberg Depression Rating Scale (MADRS) Total Score: LS Mean (+/−SE) of Changes Over Time-ANCOVA LOCF; Double-blind Treatment Phase for the full efficacy analysis set. LS mean and SE were based on analysis of covariance (ANCOVA) model with treatment (placebo, esketamine 84 mg), analysis center, standard of care antidepressant treatment as randomized (antidepressant monotherapy, antidepressant plus augmentation therapy) as factors and baseline value as a covariate. A negative change in score indicates improvement.

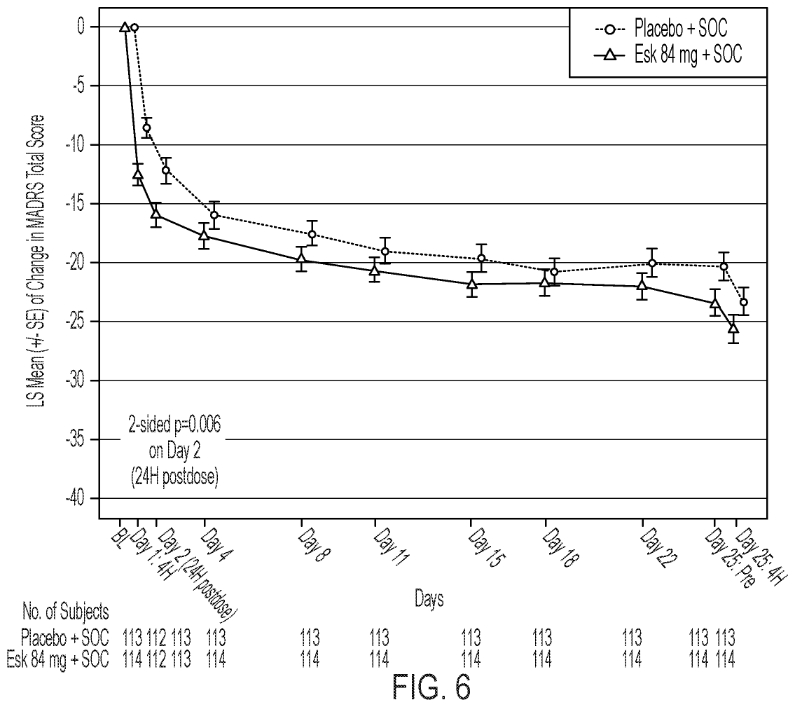

FIG. 7 is a bar graph showing the Clinical Global Impression-Severity of Suicidality-Revised (CGI-SS-R) Score: Frequency Distribution at Baseline, 4 Hours Post First Dose, 24 Hours Post First Dose and Day 25; LOCF; Double-blind Treatment Phase for the full efficacy analysis set.

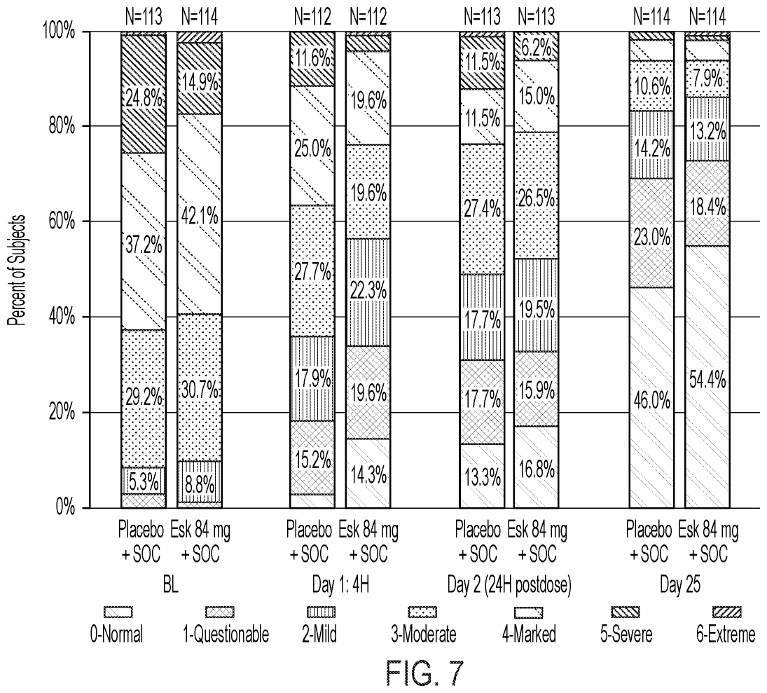

FIG. 8 is a line graph showing the Montgomery-Asberg Depression Rating Scale (MADRS) total score: LS Mean (+/−SE) of changes over time; the MMRM observed case; double-blind treatment phase for the full efficacy analysis set. LS mean and SE were based on MMRM analysis with treatment (placebo, esketamine 84 mg), time, analysis center, standard of care antidepressant treatment as randomized (antidepressant monotherapy, antidepressant plus augmentation therapy), time by treatment interaction as factors and baseline value as a covariate; Negative change in score indicates improvement.

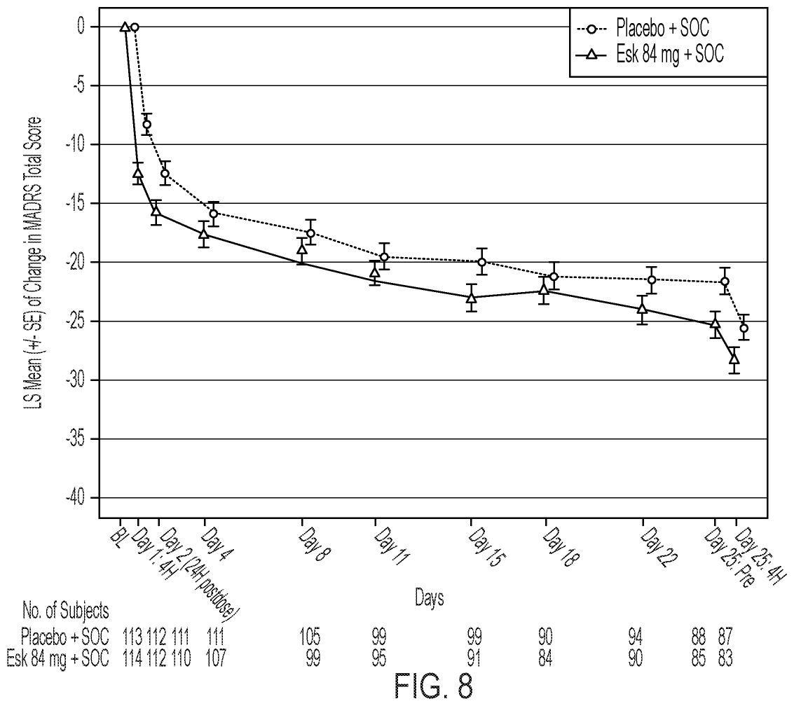

FIG. 9 is a forest plot for the MADRS-LS mean treatment difference of change from baseline to 24 hours post first dose for Example 4.

FIG. 10 is a forest plot for the MADRS-LS mean treatment difference of change from baseline to 24 hours post first dose for Example 5.

FIG. 11 is a forest plot for the MADRS at 24 hours post first dose: subgroup analyses pooled analysis for Examples 4 and 5.

FIG. 12 is a forest plot for the CGI-SS-R-treatment difference of change from baseline to 24 hours post-first dose for Example 4.

FIG. 13 is a forest plot for the CGI-SS-R-treatment difference of change from baseline to 24 hours post-first dose for Example 5.

FIG. 14 is a forest plot for the CGI-SS-R at 24 hours post first dose: subgroup analyses for Examples 4 and 5.

FIG. 15 is a bar graph showing MADRS remission by prior suicide attempt status—pooled analysis for Examples 4 and 5 for patients with a history of suicide attempts (MADRS remission refers to a MADRS total score ≤12).

FIG. 16 is a bar graph showing MADRS remission by prior suicide attempt status—pooled analysis for Examples 4 and 5 for patients without a history of suicide attempts (MADRS remission refers to a MADRS total score ≤12).

FIG. 17 is a forest plot for the prior suicide attempt sub-group analysis using CGI-SS-R for Examples 4 and 5.

FIG. 18 is a bar graph showing MADRS remission rates (MADRS total score ≤12) over time during the DB treatment phase (fully efficacy analysis set) for Example 4.

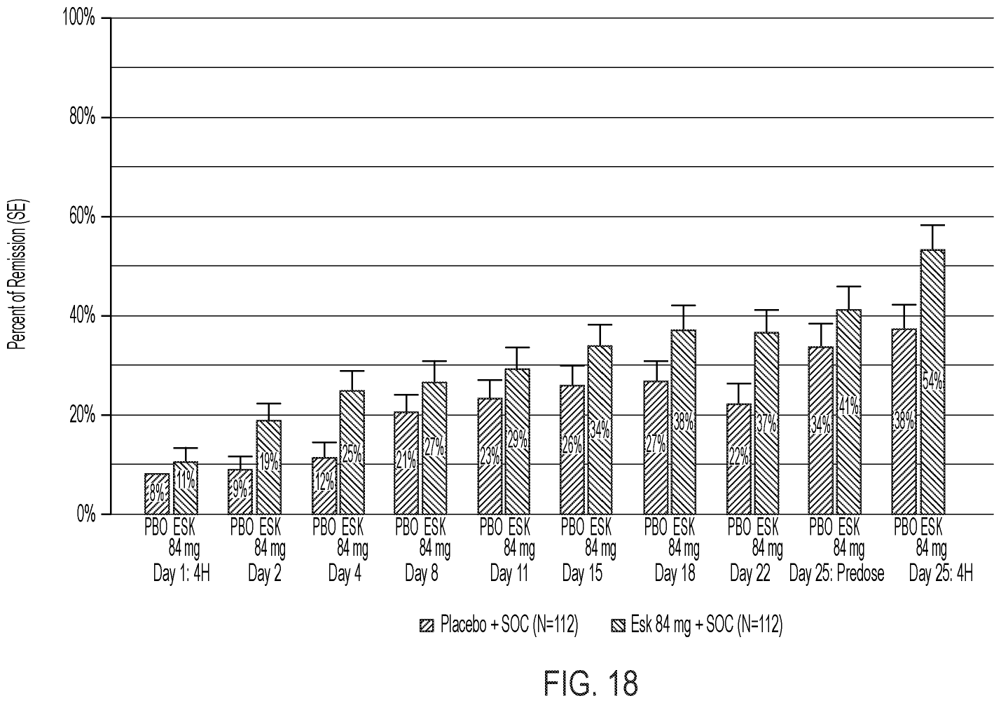

FIG. 19 is a bar graph showing MADRS remission rates (MADRS total score ≤12) over time during the DB treatment phase (fully efficacy analysis set) for Example 5.

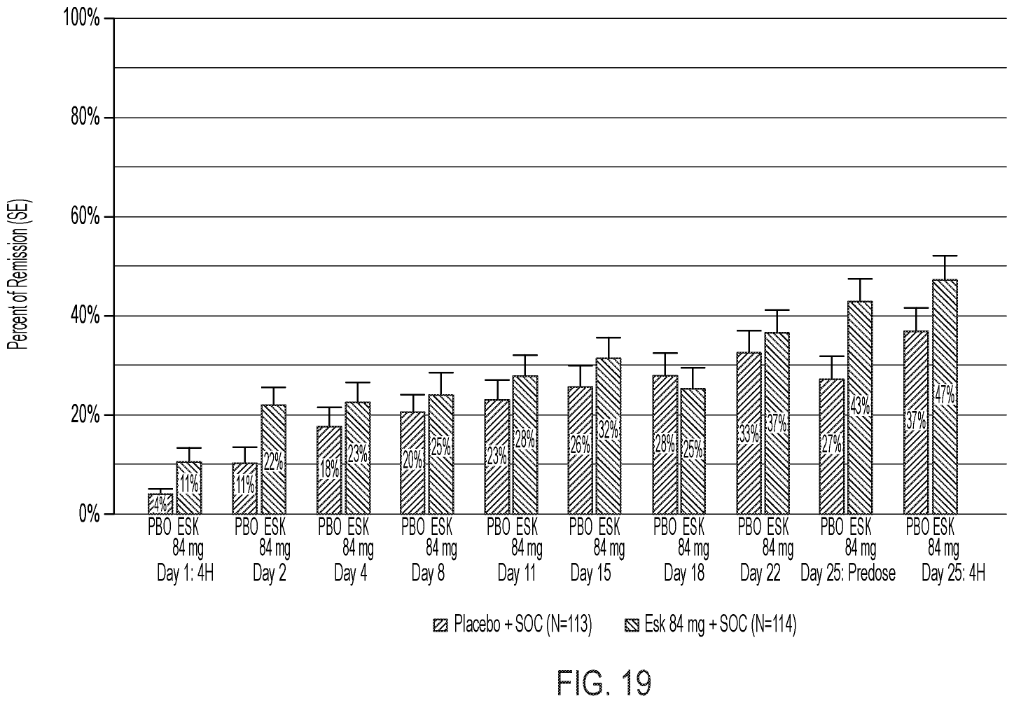

FIG. 20 is a forest plot showing the odds ratios for improved scores on the CGI-SS-R and other suicidality indices at 4 hours, 24 hours post first dose and Day 25 (IRT; LOCF; DB treatment phase (full efficacy analysis set) for Example 4. Findings of all indices of suicidality (CGI-SS-R, the MADRS suicidal thoughts item, CGI-SR-I, clinician-rated FoST, and patient-reported FoST) at 4 hours and 24 hours post first dose and Day 25, based on the IRT model are provided.

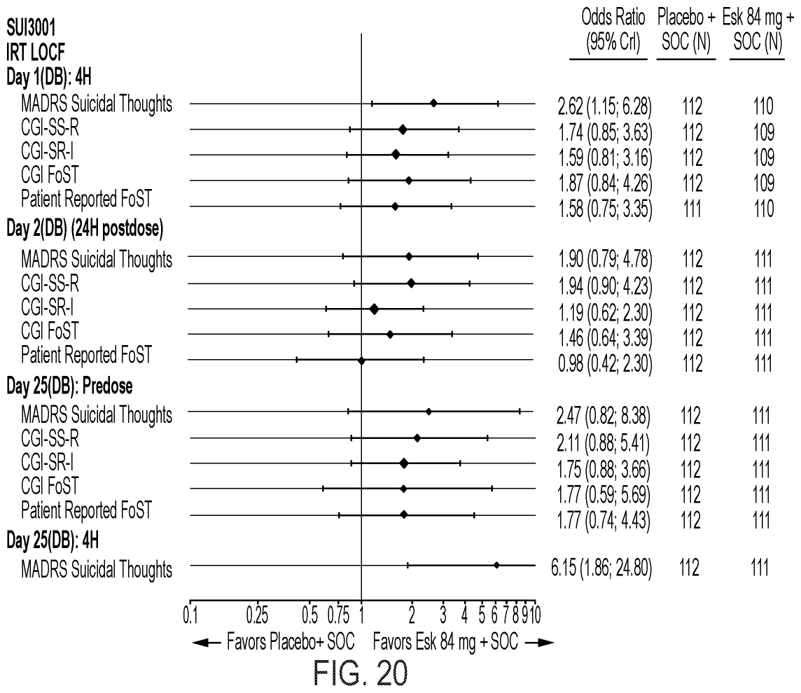

FIG. 21 is a forest plot showing the odds ratios for improved scores on the CGI-SS-R and other suicidality indices at 4 hours, 24 hours post first dose and Day 25 (IRT; LOCF; DB treatment phase (full efficacy analysis set) for Example 5. Findings of all indices of suicidality (CGI-SS-R, the MADRS suicidal thoughts item, CGI-SR-I, clinician-rated FoST, and patient-reported FoST) at 4 hours and 24 hours post first dose and Day 25, based on the IRT model are provided.

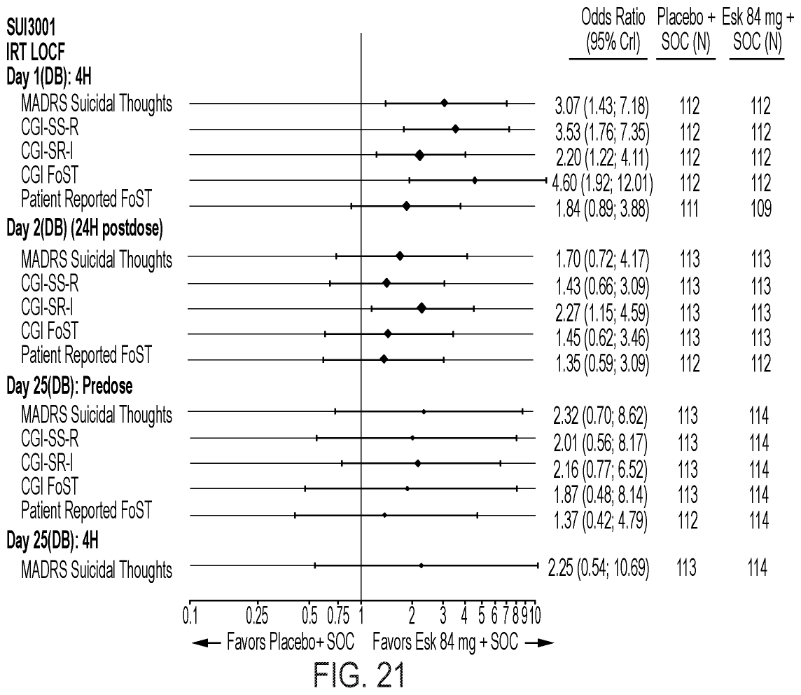

### DETAILED DESCRIPTION OF ILLUSTRATIVE EMBODIMENTS

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “esketamine” shall mean the (S)-enantiomer of ketamine, a compound of formula (I):

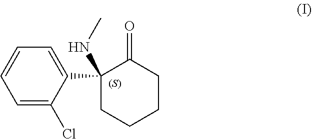

also known as (S)-2-(2-chlorophenyl)-2-(methylamino)cyclohexanone. “Esketamine” shall also mean a salt, e.g., a chloride salt such as the hydrochloride salt, of the (S)-enantiomer of ketamine, i.e., a compound of formula (II):

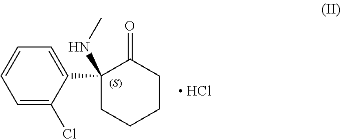

also known as (S)-2-(2-chlorophenyl)-2-(methylamino)cyclohexanone hydrochloride.
In some embodiments, the esketamine is substantially free of the (R)-enantiomer of ketamine, i.e. a compound of formula (III):

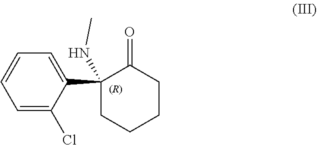

In other embodiments, the esketamine contains less than about 10% by weight, based on the weight of the esketamine sample, of the (R)-enantiomer of ketamine. In further embodiments, the esketamine contains less than about 10, 9, 8, 7, 6, 5, 4, 3, 2, 1, 0.5, 0.1, 0.005, or 0.001% by weight, based on the weight of the esketamine sample, of the (R)-enantiomer of ketamine. In yet other embodiments, the esketamine contains about 0.001 to about 10% by weight, based on the weight of the esketamine sample, of the (R)-enantiomer of ketamine. In still further embodiments, the esketamine contains about 0.001 to about 10%, about 0.001 to about 5%, about 0.001 to about 1, about 0.001 to about 0.5, about 0.001 to about 0.1, about 0.1 to about 5, about 0.1 to about 1, about 0.1 to about 5, or about 0.5 to about 5% by weight, based on the weight of the esketamine sample, of the (R)-enantiomer of ketamine.

The term “esketamine” may also include other pharmaceutically acceptable salts thereof, which may readily be selected by those skilled in the art. A “pharmaceutically acceptable salt” is intended to mean a salt of esketamine that is non-toxic, biologically tolerable, or otherwise biologically suitable for administration to the subject. See, generally, G. S. Paulekuhn, “Trends in Active Pharmaceutical Ingredient Salt Selection based on Analysis of the Orange Book Database”, J. Med. Chem., 2007, 50:6665-72, S. M. Berge, “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for administration to patients without undue toxicity, irritation, or allergic response.

Examples of other pharmaceutically acceptable salts include sulfates, pyrosulfates, bisulfates, sulfites, bisulfites, phosphates, monohydrogen-phosphates, dihydrogenphosphates, metaphosphates, pyrophosphates, bromides (such as hydrobromides), iodides (such as hydroiodides), acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates. In particular, the salt of esketamine is a hydrochloride salt.

Unless otherwise noted, the amounts of esketamine described herein are set forth on an esketamine free base basis. That is, the amounts indicate that amount of the esketamine molecule administered, exclusive of, for example, counterions (such as in pharmaceutically acceptable salts).

In certain embodiments, the esketamine is administered intranasally. In other embodiments, the esketamine is administered intranasally as its corresponding hydrochloride salt. In further embodiments, the esketamine is administered intranasally as its corresponding hydrochloride salt in an 16.14% weight/volume solution (equivalent to 14% weight/volume of esketamine base). In yet other embodiments, the esketamine is administered intranasally as a solution comprising 161.4 mg/mL of esketamine hydrochloride (equivalent to 140 mg/mL of esketamine base), 0.12 mg/mL of ethylenediaminetetraacetic acid (EDTA) and 1.5 mg/mL citric acid, at a pH of 4.5 in water. In still further embodiments, the esketamine is administered intranasally, wherein the intranasal delivery administers 100 μL of a solution comprising 161.4 mg/mL of esketamine hydrochloride (equivalent to 140 mg/mL of esketamine base), 0.12 mg/mL of ethylenediaminetetraacetic acid (EDTA) and 1.5 mg/mL citric acid, at a pH of 4.5 in water. In other embodiments, the esketamine is delivered intranasally using a nasal spray pump, wherein the pump delivers 100 μL of a solution comprising 161.4 mg/mL of esketamine hydrochloride (equivalent to 140 mg/mL of esketamine base), 0.12 mg/mL of ethylenediaminetetraacetic acid (EDTA) and 1.5 mg/mL citric acid, at a pH of 4.5 in water.

In general, a single pump from a nasal spray device may be configured to deliver about 50 μL to about 200 μL of an esketamine solution to a nostril of the subject, including about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, and about 200 μL. Accordingly, two pumps deliver about 100 μL to about 400 μL to the subject.

As used herein, the term “depression” includes major depressive disorder, persistent depressive disorder, seasonal affective disorder, postpartum depression, premenstrual dysphoric disorder, situational depression, anhedonia, melancholy, mid-life depression, late-life depression, depression due to identifiable stressors, treatment resistant depression, or combinations thereof. In certain embodiments, the depression is major depressive disorder (see criteria for major depression as specified in the Diagnostic and statistical Manual of Mental Disorders, 5th Edition: DSM 5). In other embodiments, the major depressive disorder is with melancholic features or anxious distress. In further embodiments, the depression is treatment-resistant depression.

As used herein, “suicide”, also known as completed suicide, is the “act of taking one's own life”. See, http://en.wikipedia.org/wiki/Suicide-cite_note-7. “Attempted suicide” or non-fatal suicidal behavior is self-injury with the desire to end one's life that does not result in death. Suicide attempt is a self-initiated sequence of behaviors by an individual who at the time of initiation, expected that the set of actions would lead to his or her own death. Risk factors for suicide include, but are not limited to: previous attempt(s), anhedonia, concurrent mental disorders, substance abuse, serious or chronic health conditions, low level of social support (e.g. living alone), expressed feelings of hopelessness or triggering stressful life event (e.g. death, divorce, separation, job loss, significant financial reversal).

As used herein, “suicidality” refers to one or more of the following: recurrent thoughts of death (not just fear of dying), recurrent suicidal ideation without a specific plan, a suicide attempt, or a specific plan for committing suicide.

As used herein, “suicidal ideation” refers to thoughts about or an unusual preoccupation with suicide, or thoughts of ending one's life or not wanting to live anymore but not necessarily taking any active efforts to do so. The range of suicidal ideation varies greatly from fleeting to chronic and progress to detailed planning, role playing, and unsuccessful attempts, which may be deliberately constructed to fail or be discovered, or may be fully intended to result in death. “Suicidal ideation with intent” may be confirmed through questioning of the patient in view of the scales/tools disclosed herein, and includes thinking (even momentarily) about harming or of hurting or of injuring oneself, with at least some intent or awareness that you might die as a result; or thinking about suicide (i.e., about killing oneself, and intending to act on thoughts of killing oneself).

Certain scales/tools may be used in the evaluation of suicidality and/or suicidal ideation, including the Beck Scale for Suicide Ideation (BSS), Columbia Suicide Severity Rating Scale (C-SSRS), Suicidal Ideation and Behavioral Assessment Tool (SIBAT), the Clinical Global Impression-Severity of Suicidality-Revised (CGI-SS-R), the Mini-International Neuropsychiatric Interview (MINI), and the Frequency of Suicidal Thinking (FoST).

As used herein “imminent risk of suicide” refers to a patient with high levels of suicidal ideation, intent to act on their suicidal ideation and current suicidal capabilities to do harm to themselves who would very likely do serious harm or killing him/her self in the immediate future. Immediate future is a short-defined period of time usually less than 2 weeks, less than 1 week, less than 2 days, less than 1 day or less than a few hours.

The methods described herein are appropriate for patients assessed to be at imminent risk for suicide. This assessment is typically made by a treating physician or other qualified health care professional or clinician. This assessment may be aided by use of the scales/tools noted herein and include the health care professional's overall experience with the patient and the patient's medical records and history. For example, an assessment of imminent risk may be made where the patient has regular ideations with intent or potential for impulsive actions for suicide with or without a plan or recent attempts; frequent ideations with intent and/or well worked out suicide plan with or without recent suicide attempt; or nearly constant suicidal ideations and intent and/or a well worked out plan and preparations underway or recent attempt. The assessment of imminent risk may be confirmed by asking the patent questions, such as whether or not they think about suicide and whether or not they intend to act on thoughts of suicide.

As used herein, the term “standard of care treatment” refers to a physician-prescribed treatment for a patient suffering from major depressive disorder, including patients with suicidality and/or suicidal ideation, that have been assessed to be at imminent risk of suicide. For purposes of this disclosure, unless otherwise noted, the standard of care treatment does not include esketamine.

In some aspects, the standard of care treatment comprises, consists of, or consists essentially of the standard of care treatment(s) disclosed in the examples herein. In certain aspects, the standard of care treatment includes in-patient psychiatric hospitalization and the initiation or optimization of standard antidepressant medication (determined by the treating physician based on clinical judgment and practice guidelines). The standard of care may also include other concomitant medication, e.g., benzodiazepines, without prohibiting any psychotherapies. In other embodiments, the standard of care further includes outpatient treatment following discharge from the initial hospitalization. The frequency and duration of outpatient treatment may be proscribed by a treating physician or other healthcare professional, and includes, for example, once weekly, twice weekly, thrice weekly, or more visits, for up to 1 week, up to two weeks, up to three weeks, up to four weeks, or up to five weeks or more in duration. As disclosed in the examples, such outpatient treatment may occur twice weekly, for two-plus hours each, in an out-patient facility or program. Outpatient psychiatric care may include one or more of the following: psychiatric evaluation, medical management, group therapy, family intervention, neuropsychological testing, psychotherapy, among others.

It should be noted that the standard of care treatment disclosed in the examples constituted a clinical-trial level of care. Patients may not receive the same level of care outside of the clinical trial setting. Thus, the results of the trials reflected in the examples should be viewed in that context. For example, reported differences between treatment groups should be considered in the context of the trial, including the substantial beneficial effects of, for example, in-patient psychiatric hospitalization and/or concomitant outpatient treatment visits, in diffusing the acute suicidal crisis in the participants in the treatment groups. In studying such a high-risk patient population, clinical trial protocols are enhanced or more comprehensive to ensure ethical practice and patient safety.

Typically, the standard of care treatment is provided as proscribed by the treating physician or other health care profession and is concomitant with the esketamine treatment, e.g., standard of care treatment is provided during the same treatment period as the esketamine treatment. The standard of care treatment may also precede treatment with esketamine and may continue after treatment with esketamine is discontinued. With respect to antidepressants used in the standard of care treatment, the esketamine and the antidepressant(s) may be administered via the same or different routes of administration. Examples of suitable methods of administration include, but are not limited to, oral, intravenous (iv), intranasal (in), intramuscular (im), subcutaneous (sc), transdermal, buccal, or rectal. In preferred embodiments, esketamine is administered intranasally.

As used herein, unless otherwise noted, the term “antidepressant” shall mean any pharmaceutical agent which can be used to treat depression. Suitable examples include, without limitation, a mono-amine oxidase inhibitor, tricyclic, serotonin reuptake inhibitor, serotonin noradrenergic reuptake inhibitor, or noradrenergic and specific serotonergic agent. Other examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, clomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, citalopram, escitalopram, fluvoxamine, and the like; serotonin receptor antagonists such as nefazadone, and the like; serotonin noradrenergic reuptake inhibitors such as venlafaxine, milnacipran, desvenlafaxine, duloxetine and the like; noradrenergic and specific serotonergic agents such as mirtazapine, and the like; noradrenaline reuptake inhibitors such as reboxetine, edivoxetine and the like; natural products such as Kava-Kava, St. John's Wort, and the like; dietary supplements such as s-adenosylmethionine., and the like; and neuropeptides such as thyrotropin-releasing hormone and the like; compounds targeting neuropeptide receptors such as neurokinin receptor antagonists and the like; and hormones such as triiodothyronine, and the like. In some embodiments, the antidepressant is imipramine, amitriptyline, desipramine, nortriptyline, doxepin, protriptyline, trimipramine, maprotiline, amoxapine, trazodone, bupropion, clomipramine, fluoxetine, duloxetine, escitalopram, citalopram, sertraline, paroxetine, fluvoxamine, nefazadone, venlafaxine, milnacipran, reboxetine, mirtazapine, phenelzine, tranylcypromine, moclobemide, Kava-Kava, St. John's Wart, s-adenosylmethionine, thyrotropin releasing hormone, a neurokinin receptor antagonist, or triiodothyronine. Preferably, the antidepressant is selected from the group consisting of fluoxetine, imipramine, bupropion, venlafaxine and sertraline.

Therapeutically effective dosage levels and dosage regimens for antidepressants (for example, mono-amine oxidase inhibitors, tricyclics, serotonin reuptake inhibitors, serotonin noradrenergic reuptake inhibitors, noradrenergic and specific serotonergic agents, noradrenaline reuptake inhibitor, natural products, dietary supplements, neuropeptides, compounds targeting neuropeptide receptors, hormones and other pharmaceutical agents disclosed herein), may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http:///www.pdrel.com) or other sources.

In certain instances, antidepressant therapy can be augmented with antipsychotic drugs. As used herein the term “antipsychotic” includes, but is not limited to:

(a) typical or traditional antipsychotics, such as phenothiazines (e.g., chlorpromazine, thioridazine, fluphenazine, perphenazine, trifluoperazine, levomepromazin), thioxanthenes (e.g., thiothixene, flupentixol), butyrophenones (e.g., haloperidol), dibenzoxazepines (e.g., loxapine), dihydroindolones (e.g., molindone), substituted benzamides (e.g., sulpride, amisulpride), and the like; and

(b) atypical antipsychotics and mood stabilizers, such as paliperidone, clozapine, risperidone, olanzapine, quetiapine, zotepine, ziprasidone, iloperidone, perospirone, blonanserin, sertindole, ORG-5222 (Organon), and the like; and others such as sonepiprazole, aripiprazole, nemonapride, SR-31742 (Sanofi), CX-516 (Cortex), SC-111 (Scotia), NE-100 (Taisho), divalproate (mood stabilizer) and the like.

In an embodiment, the “atypical antipsychotic” is selected from the group consisting of aripiprazole, quetiapine, olanzapine, risperidone and paliperidone. In another embodiment, the atypical antipsychotic is selected from the group consisting of aripiprazole, quetiapine, olanzapine and risperidone; preferably, the atypical antipsychotic is selected from the group consisting of aripiprazole, quetiapine and olanzapine.

As used herein, the term “treatment-refractory or treatment-resistant depression” and the abbreviation “TRD” shall be defined as major depressive disorder in a patient that does not respond adequately to at least two different antidepressants, preferably between two and five antidepressants in the current depressive episode. In other embodiments, TRD is defined as major depressive disorder in a patient that has not responded to at least two oral antidepressants of adequate dose and duration in the current depressive episode. For those patients that do not respond to the present methods of treatment, there is an option to further monitor and treat patients with additional therapies, including therapies for treatment resistant depression. For example, esketamine has been approved for treating treatment resistant depression, and such approved methods may be utilized as proscribed by the treating physician or other health care professional. Methods for treating treatment resistant depression are disclosed, for example, in U.S. Patent Application Publication No. 2016/0074340, and International Patent Application Publication No. WO/2019/126108, both of which are incorporated by reference herein.

One skilled in the art will recognize that the failure to respond to an adequate course of a given antidepressant may be determined retrospectively or prospectively. In an embodiment, at least one of the failures to respond to an adequate course of antidepressant is determined prospectively. In another embodiment, at least two of the failures to respond to an adequate course of antidepressant are determined prospectively. In another embodiment, at least one of the failures to respond to an adequate course of antidepressant is determined retrospectively. In another embodiment, at least two of the failures to respond to an adequate course of antidepressant are determined retrospectively in a current depressive episode.

As used herein, unless otherwise noted, the terms “treating”, “treatment” and the like, shall include the management and care of a human patient for the purpose of combating a disease, condition, or disorder and includes, for example, the administration of a compound described herein to prevent the onset of one or more of the symptoms or complications, alleviate one or more of the symptoms or complications, or eliminate the disease, condition, or disorder.

The term “therapeutically effective amount” as used herein, means that amount of active compound or pharmaceutical agent that elicits the biological or medicinal response in a human that is being sought by a medical doctor or other clinician, which includes alleviation of one or more of the symptoms of the disease or disorder being treated. In some embodiments of the present disclosure, the therapeutically effective amount of esketamine is about 20 to about 100 mg. In other embodiments, the therapeutically effective amount is about 28 to about 84 mg. In further embodiments, the therapeutically effective amount is about 56 to about 84 mg. In yet other embodiments, the therapeutically effective amount is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg. In further embodiments, the therapeutically effective amount is about 28 mg. In other embodiments, the therapeutically effective amount is about 56 mg. In still further embodiments, the therapeutically effective amount is about 84 mg.

In some embodiments, the patient is an adult. The term “adult” as used herein refers to a human that is about 18 years of age or older.

### Methods

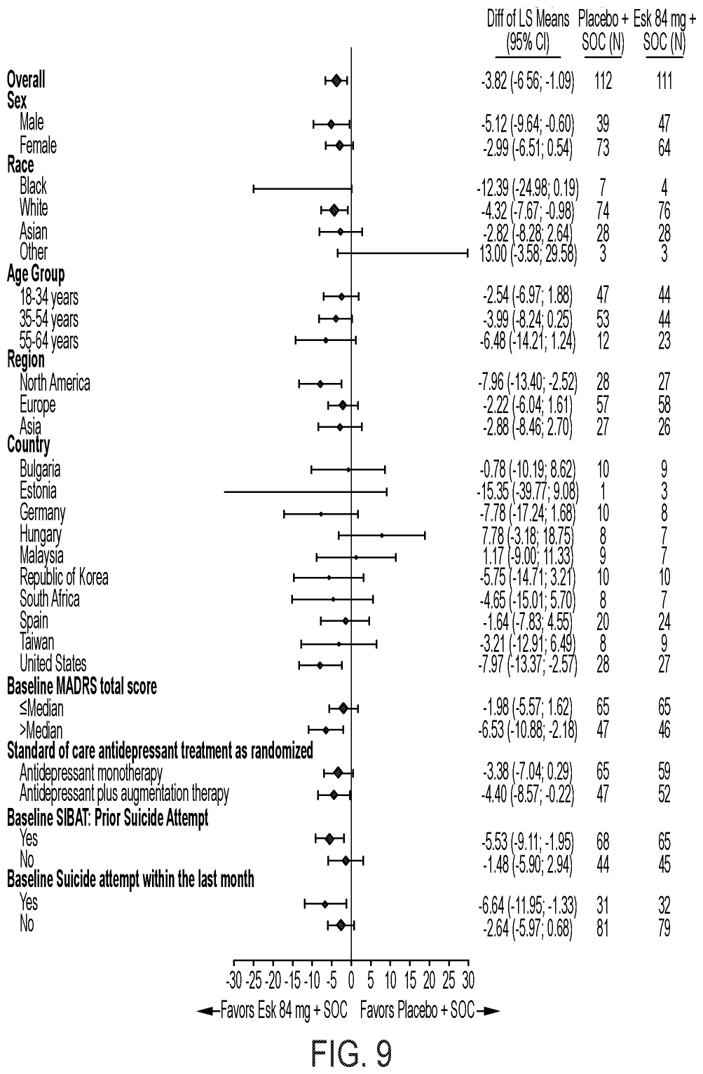

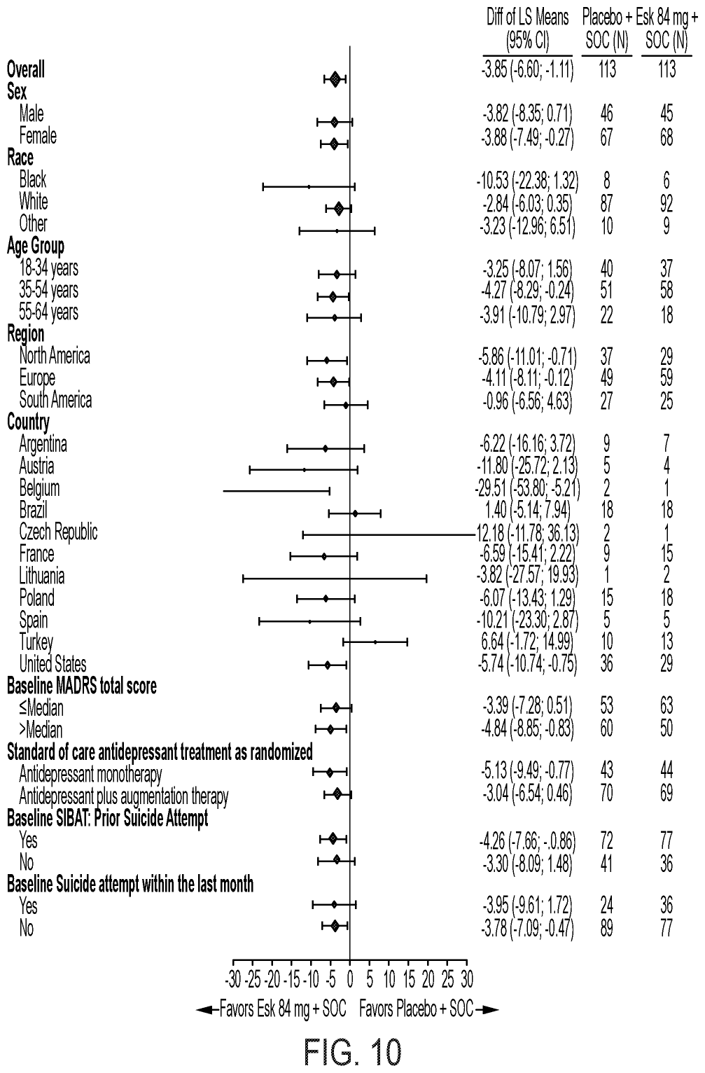

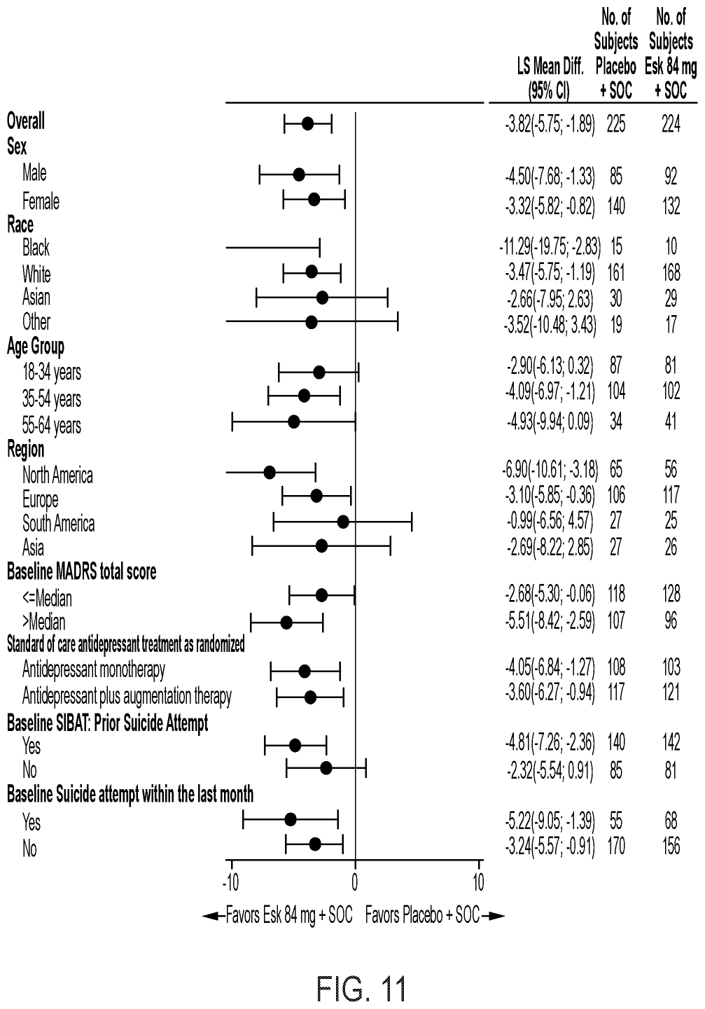

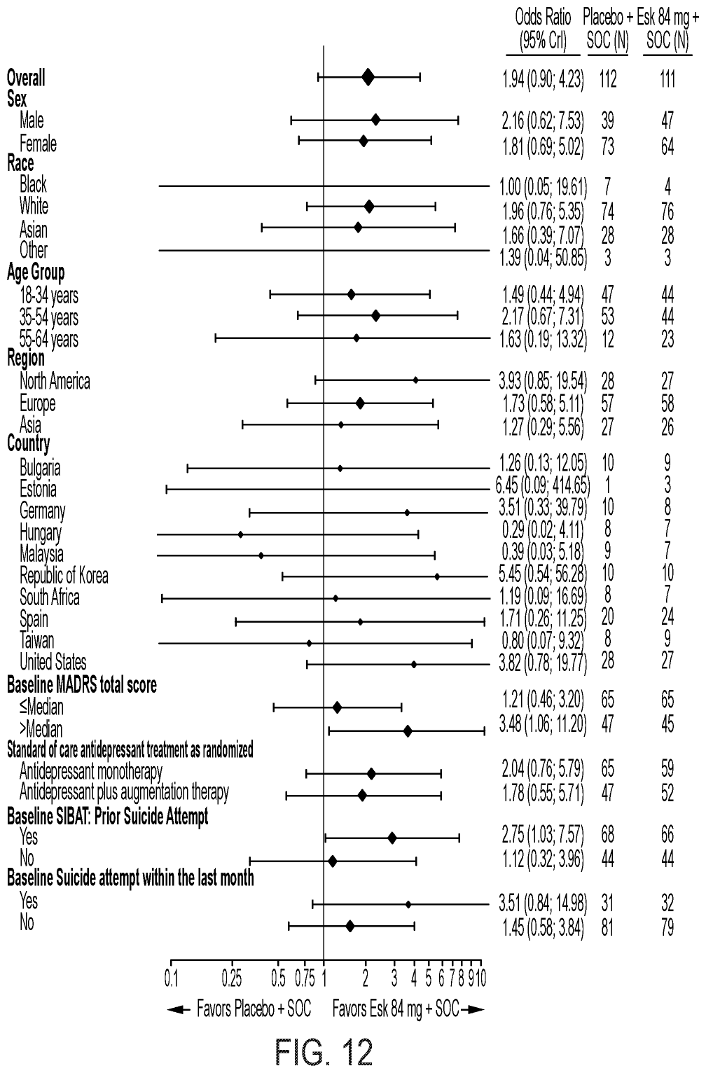

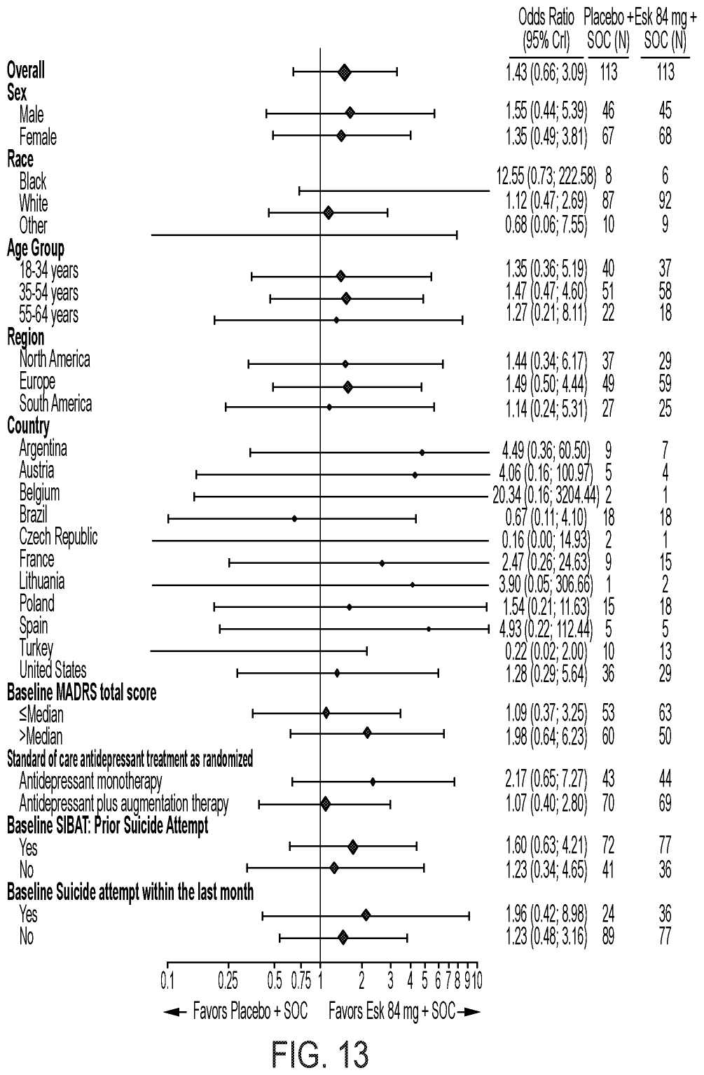

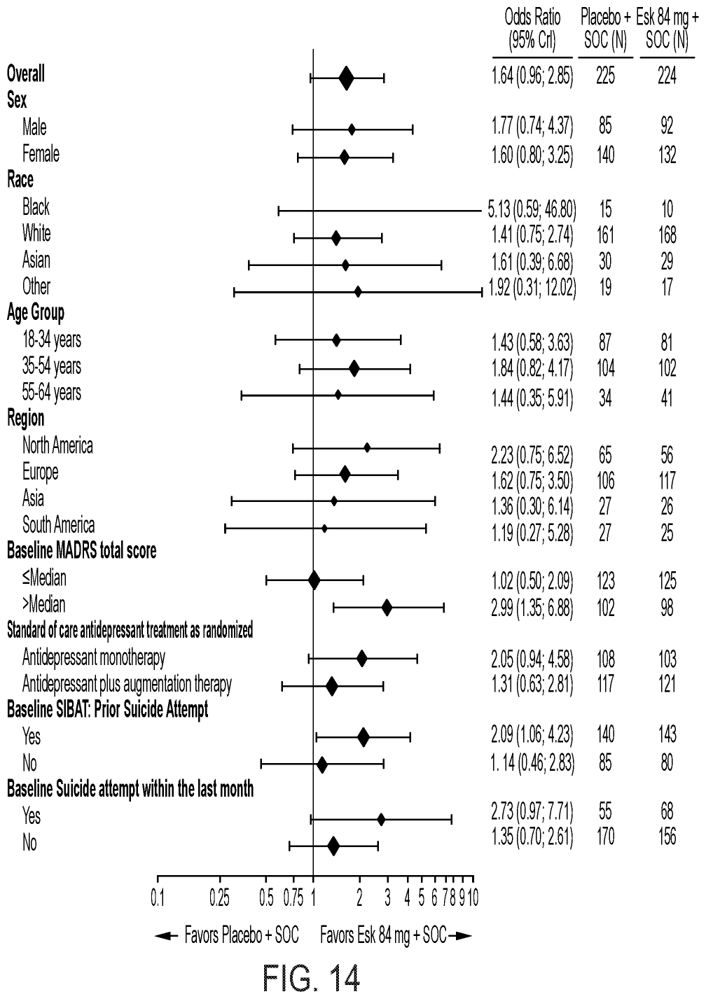

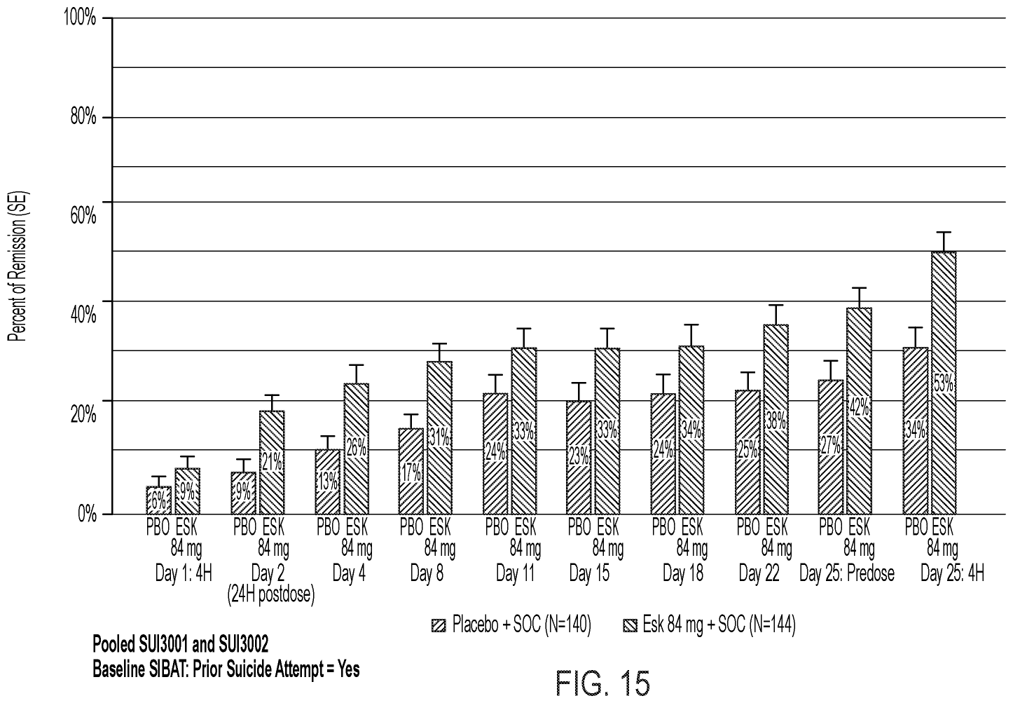

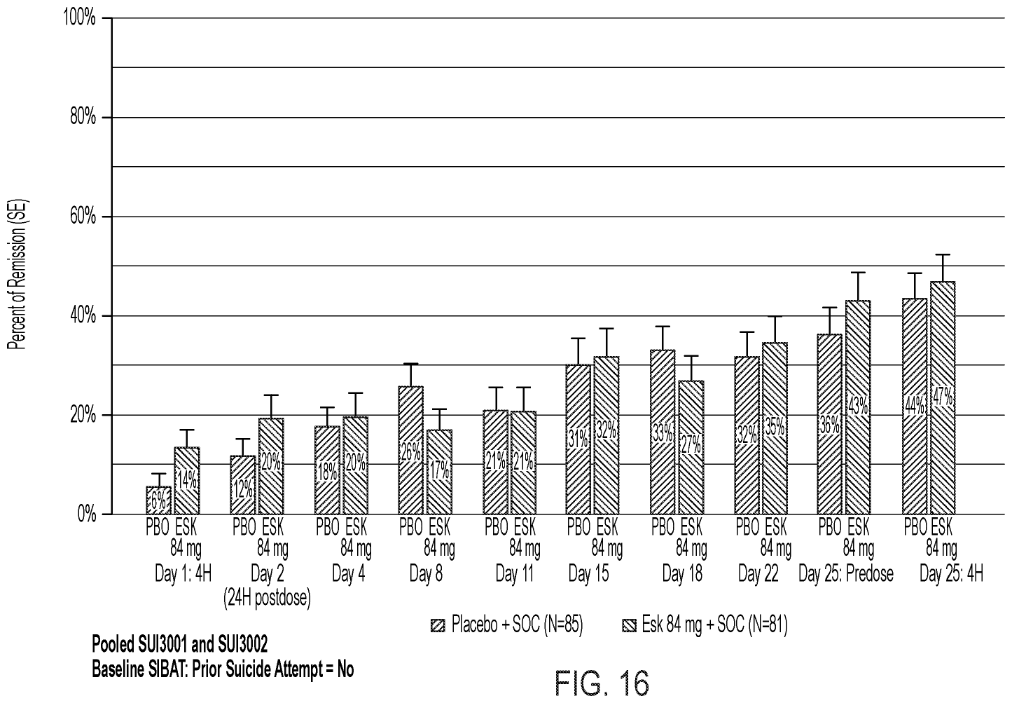

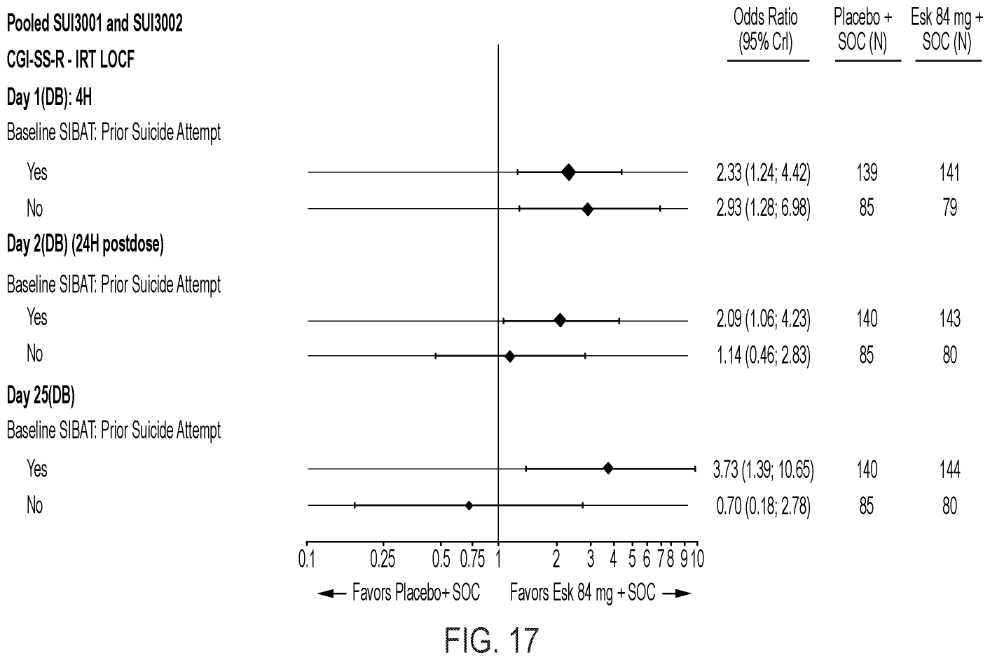

In certain aspects, the disclosure relates to methods for reducing symptoms of major depressive disorder, including suicidality, in a human patient assessed to be at imminent risk for suicide comprising the administration of esketamine in addition to standard of care treatment. As reflected in the reported data, for depressive symptoms, the treatment differences favored the administration of esketamine plus standard of care treatment over standard of care treatment alone. In addition, treatment differences between patients that previously attempted suicide (attempters) and those that had not (non-attempters) were also present (see, e.g., data reflected in FIGS. 9-17). For example, FIG. 11 shows the estimated differences (95% CI) between the esketamine+SOC and placebo+SOC treatment groups for the change in MADRS total score at 24 hours after the first dose were −4.81 (−7.26; −2.36) for the subpopulation that reported a prior suicide attempt and −2.32 (−5.54; 0.91) for the subpopulation that did not report a prior suicide attempt. FIG. 17 shows the odds ratio (95% CrI) for an improved CGI-SS-R score at 24 hours after the first dose of esketamine+SOC relative to placebo+SOC was 2.09 (1.06; 4.23) in the subpopulation of subjects with a history of prior suicide attempt and 1.14 (0.46; 2.83) for the subpopulation that did not report a prior suicide attempt. Thus, the results showed that esketamine+SOC appeared to benefit, in particular, the subpopulation of patients with prior suicide attempt. Improvement in this population is of clinical relevance considering that prior suicide attempt has been identified as the single most important predictor/risk factor for suicide in subjects with MDD.

The subpopulation analyses also indicated that attempters showed less treatment benefit in connection with standard of care treatment than did non-attempters. As a result, physicians may consider options for treating attempters in view of an enhanced benefit profile. For example, patients with MDD presenting with suicidal ideation with intent constitute a psychiatric emergency that requires immediate intervention. Given the lower treatment benefit shown with standard of care treatment alone for attempters, the benefit of including an esketamine treatment for such patients may outweigh the risks associated with esketamine. In contrast, non-attempters may not present with the same benefit profile.

We believe that in a real world clinical care setting that attempters will retain an enhanced benefit profile from the administration of esketamine. As such, it is expected that the attempter population will show greater reductions in their depressive symptoms of MDD as defined in DSM-5. Additionally, this population may have reduced levels of suicidality for example either by reduced levels of suicidal ideation and/or suicide attempts.

Accordingly, in certain embodiments, the methods comprise determining if the patient has previously attempted suicide, and, if so, treating such patient with a standard of care treatment and a therapeutically effective amount of esketamine. In other aspects, to the extent a patient is determined not to have previously attempted suicide, the patient is administered standard of care treatment without treating the patient with esketamine. Determining whether or not a patient had previous suicide attempt may be done by the treating physician or other health care professional by way of questioning or interviewing the patient or individuals familiar with the patient's medical history and/or consultation of medical records.

In embodiments including esketamine and standard of care treatment, the methods comprise administering a therapeutically effective amount of esketamine at a given frequency of at least twice a week over a treatment period in which the patient is shown to be responsive to the treatment. At timepoints therein, the patient's condition may be assessed by a treating physician or other healthcare professional. In some embodiments, the treatment period is a period of about 1 to about 4 weeks. In other embodiments, the induction phase is a period of up to about 1 week, up to about 2 weeks, up to about 3 weeks, or up to about 4 weeks.

In preferred embodiments, the methods comprise intranasally administering about 56 mg to about 84 mg of esketamine per treatment session, wherein the treatment session occurs at a frequency of twice weekly for a treatment period that has a duration of about 4 weeks. In certain embodiments, about 84 mg of esketamine is administered per treatment session. Depending on tolerability of the about 84 mg dose, the dose at subsequent treatment sessions may remain at about 84 mg or be reduced to about 56 mg. In certain aspects, the esketamine is delivered from an intranasal administration device in 2 or more sprays, more preferably 4 to 6 sprays.

A representative nasal spray device is disclosed in U.S. Pat. Nos. 6,321,942; 7,299,949; and 9,555,950; and U.S. patent application Ser. No. 16/440,570, all of which are incorporated by reference herein. For example, a disposable atomizer for discharging successive partial discharge amounts as a spray may be utilized to carry out the methods discloses herein. Typically, such devices allow a medicament to be sprayed into both nostrils of a patient in two successive strokes. The device may be ready-to-use wherein the medicament is discharged from a medium container. The device is typically able to separate a first discharge stroke from a second discharge stroke to prevent complete emptying of the medium container in a single motion. The device may take the form of a double-stroke disposable pump, which is disposed of after a single use and enables individual partial discharges with high dosing precision and reliability.

In one embodiment, the nasal spray device is a single-use device that delivers a total of 28 mg of esketamine in two sprays, one spray per nostril. The device may be operated by the patient under the supervision of a healthcare professional. With respect to dosage amounts, one device may be used for a 28 mg dose, two devices for a 56 mg dose, or three devices for an 84 mg dose. It is also preferable to have a 5-minute interval between the use of each device. As described in Tables 2 and 3 in the example section, time 0 is defined as the time of administration of the first intranasal spray to one nostril from the first intranasal device. The designated dose of esketamine is administered in a treatment session. For example, the “treatment session” for 56 mg of esketamine may include 2 sprays from a first device and 2 sprays from a second device. As another example, the “treatment session” for 84 mg of esketamine may include 2 sprays from a first device (1 spray to each nostril), 2 sprays from a second device (1 spray to each nostril), and 2 sprays from a third device (1 spray to each nostril). However, more devices may be as needed, e.g., if a device fails to operate properly and additional devices are required to administer the required dosage or amount of esketamine. The treatment session typically begins when the first spray is administered to one nostril from the first device. The treatment session ends when the last spray is administered to a nostril from the last device.

The term “twice weekly” as used herein refers to a frequency that is two times in a weekly (7-day) period. For example, “twice weekly” may refer herein to the administration of esketamine. “Twice weekly” may also refer to a frequency of monitoring a patient, including outpatient visits, as disclosed herein. In some embodiments, twice weekly refers to a frequency that is day 1 and day 2 of a week. In other embodiments, twice weekly refers to a frequency that is day 1 and day 3 of a week. In further embodiments, twice weekly refers to a frequency that is day 1 and day 4 of a week. In still other embodiments, twice weekly refers to a frequency that is day 1 and day 5 of the week. The “day 1” may be any day of the week, including, Sunday, Monday, Tuesday, Wednesday, Thursday, Friday, or Saturday. Typically, with respect to administration of esketamine, twice weekly refers to a frequency that is day 1 and day 4 of a week. To the extent there is a mis-dose, the dose may be taken as soon as possible thereafter and the prescribed regimen thereafter continued.

### Compositions and Methods for Preparing

As used herein, the term “composition” is intended to encompass a product comprising the specified ingredients in the specified amounts, as well as any product which results, directly or indirectly, from combinations of the specified ingredients in the specified amounts.

In some preferred pharmaceutical compositions, S-ketamine hydrochloride as the active ingredient is intimately admixed with a pharmaceutical carrier, preferably water, according to conventional pharmaceutical compounding techniques, which carrier may take a wide variety of forms depending of the form of preparation desired for administration. Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

One suitable aqueous formulation of S-ketamine, comprises water and S-ketamine; wherein the S-ketamine is present in an amount in the range of from about 25 mg/mL to about 250 mg/mL, preferably about 55 mg/mL to about 250 mg/mL or about 100 mg/mL to about 250 mg/mL, or any amount or range therein, based on the total volume of the pharmaceutical composition. Preferably, the S-ketamine is present in an amount in the range of from about 150 mg/ml to about 200 mg/mL, or any amount or range therein. More preferably, the S-ketamine is present in an amount in the range of from about 150 mg/mL to about 175 mg/mL, or any amount or range therein. More preferably, the S-Ketamine is present in an amount in the range of from about 160 mg/mL to about 163 mg/mL, for example, in an amount of about 161.4 mg/mL

Another suitable aqueous formulation of S-ketamine, comprising water and S-ketamine; wherein the S-ketamine is present in an amount in the range of from about eq. 100 mg/mL to about eq. 250 mg/mL, or any amount or range therein, based on the total volume of the pharmaceutical composition. Preferably, the S-ketamine is present in an amount in the range of from about eq. 125 mg/ml to about eq. 180 mg/mL, or any amount or range therein. More preferably, the S-ketamine is present in an amount in the range of from about eq. 140 mg/mL to about eq. 160 mg/mL, or any amount or range therein, for example, in an amount of about eq. 140 mg/mL.

Suitable pharmaceutical compositions for use herein are preferably an aqueous formulation. As used herein, unless otherwise noted, the term “aqueous” shall mean that the primary liquid component of the formulation is water. Preferably, water constitutes greater than about 80 wt-% of the liquid component of the pharmaceutical composition, more preferably greater than about 90 wt-%, more preferably greater than about 95 wt-%, more preferably about 98 wt-%.

In suitable pharmaceutical compositions for use herein, the water content of the composition is within the range of 85±14 wt.-%, more preferably 85±12 wt.-%, still more preferably 85±10 wt.-%, most preferably 85±7.5 wt.-% and in particular 85±5 wt.-%, based on the total weight of the composition.

In suitable pharmaceutical compositions for use herein, preferably the water content of the composition is within the range of 90±14 wt.-%, more preferably 90±12 wt.-%, still more preferably 90±10 wt.-%, most preferably 80±7.5 wt.-% and in particular 90±5 wt.-%, based on the total weight of the composition.

In another pharmaceutical composition for use herein, the water content of the composition is within the range of 95±4.75 wt.-%, more preferably 95±4.5 wt.-%, still more preferably 95±4 wt.-%, yet more preferably 95±3.5 wt.-%, most preferably 95±3 wt.-% and in particular 95±2.5 wt.-%, based on the total weight of the composition.

In a further pharmaceutical composition for use herein, the water content of the composition is within the range of from 75 to 99.99 wt.-%, more preferably 80 to 99.98 wt.-%, still more preferably 85 to 99.95 wt.-%, yet more preferably 90 to 99.9 wt.-%, most preferably 95 to 99.7 wt.-% and in particular 96.5 to 99.5 wt.-%, based on the total weight of the composition.

In yet other pharmaceutical compositions for use herein, they further comprise one or more buffers and/or buffer systems (i.e. conjugate acid-base-pairs).

As used herein, the term “buffer” shall mean any solid or liquid composition (preferably an aqueous, liquid composition) which when added to an aqueous formulation adjusts the pH of said formulation. One skilled in the art will recognize that a buffer may adjust the pH of the aqueous formulation in any direction (toward more acidic, more basic or more neutral pH). Preferably, the buffer is pharmaceutically acceptable.

Suitable examples of buffers which may be used in the aqueous formulations include, but are not limited to citric acid, sodium dihydrogen phosphate, disodium hydrogen phosphate, acetic acid, boric acid, sodium borate, succinic acid, tartaric acid, malic acid, lactic acid, furmaric acid, and the like. Preferably, the buffer or buffer system is selected from the group consisting of NaOH, citric acid, sodium dihydrogen phosphate and disodium hydrogen phosphate.

In an embodiment, the buffer is selected to adjust the pH of the S-ketamine hydrochloride pharmaceutical compositions (e.g. the aqueous formulations described herein) into a pH in the range of from about pH 3.5 to about pH 6.5, or any amount or range therein. Preferably, the buffer is selected to adjust the pH of the S-ketamine hydrochloride compositions to about in the range of from about pH 4.0 to about pH 5.5, or any amount or range therein, more preferably, in the range of from about pH 4.5 to about pH 5.0, or any amount or range therein.

Preferably, the concentration of the buffer and buffer system, respectively, preferably NaOH, is adjusted to provide a sufficient buffer capacity.

In an embodiment, pharmaceutical compositions comprising S-ketamine hydrochloride, water, and a buffer or buffer system, preferably NaOH are provided; wherein the buffer or buffer system is present in an amount sufficient to yield a formulation with a pH in the range of from about pH 4.0 to about pH 6.0, or any amount or range therein.

Optionally the pharmaceutical compositions may contain a preservative.

As used herein, unless otherwise noted, the terms “antimicrobial preservative” and “preservative” preferably refer to any substance that is usually added to pharmaceutical compositions in order to preserve them against microbial degradation or microbial growth. In this regard, microbial growth typically plays an essential role, i.e. the preservative serves the main purpose of avoiding microbial contamination. As a side aspect, it may also be desirable to avoid any effect of the microbes on the active ingredients and excipients, respectively, i.e. to avoid microbial degradation.

Representative examples of preservatives include, but are not limited to, benzalkonium chloride, benzethonium chloride, benzoic acid, sodium benzoate, benzyl alcohol, bronopol, cetrimide, cetylpyridinium chloride, chlorhexidine, chlorbutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, sodium propionate, thimerosal, methyl paraben, ethyl paraben, propyl paraben, butyl paraben, isobutyl paraben, benzyl paraben, sorbic acid, and potassium sorbate.

The complete absence of preservatives in the pharmaceutical compositions used herein is preferred when the content of S-ketamine hydrochloride is sufficiently high so that due to its preservative property the desired shelf life or in use stability can be achieved by the presence of the drug itself. Preferably, under these circumstances the concentration of S-ketamine hydrochloride is at least eq. 120 mg/mL, preferably in the range of from about eq, 120 mg/mL to about eq. 175 mg/ml, or any amount or range therein, more preferably in an amount in the range of from about eq. 125 mg/mL to about eq. 150 mg/mL, or any amount or range therein, for example at about eq. 126 mg/mL or at about eq. 140 mg/mL.

As used herein, the terms “penetration agent”, “penetration enhancer”, and “penetrant” refer to any substance that increases or facilitates absorption and/or bioavailability of the active ingredient (e.g. S-ketamine hydrochloride) of a pharmaceutical composition. Preferably, the penetration agents increases or facilitates absorption and/or bioavailability of the active ingredient (e.g. S-ketamine hydrochloride) of a pharmaceutical composition, following nasal administration (i.e. increases or facilitates absorption and/or bioavailability of the active ingredient through the mucosal membrane).

Suitable examples include, but are not limited to tetradecyl maltoside, sodium glycocholate, tauroursodeoxycholic acid (TUDCA), lecithines, and the like; and chitosan (and salts), and surface active ingredients such as benzalkonium chloride, sodium dodecyl sulfate, sodium docusate, polysorbates, laureth-9, oxtoxynol, sodium deoxycholate, polyarginine, and the like. Preferably, the penetration agent is tauroursodeoxycholic acid (TUDCA).

The penetration agent may work via any mechanism, including for example by increasing the membrane fluidity, creating transient hydrophilic pores in the epithelial cells, decreasing the viscosity of the mucus layer or opening up tight junctions. Some penetration agents (for example bile salts and fusidic acid derivatives) may also inhibit the enzymatic activity in the membrane, thereby improving bioavailability of the active ingredient.

Preferably, the penetration agent is selected to meet one or more, more preferably all, of the following general requirements:

(a) It is effective at increasing absorption (preferably nasal absorption) of the active ingredient, preferably in a temporary and/or reversible manner;

(b) It is pharmacologically inert;

(c) It is non-allergic, non-toxic and/or non-irritating;

(d) It is highly potent (effective in small amounts);

(e) It is compatible with the other components of the pharmaceutical composition;

(f) It is odorless, colorless and/or tasteless;

(g) It is accepted by regulatory agencies; and

(h) It is inexpensive and available in high purity.

In one embodiment, the penetration agent is selected to increase penetration (absorption and/or bioavailability of the S-ketamine hydrochloride) without nasal irritation. In another embodiment, the penetration agent is selected to improve absorption and/or bioavailability of the S-ketamine hydrochloride; and further selected to enhance uniform dosing efficacy.

In an embodiment, pharmaceutical compositions comprising S-ketamine and water are provided; wherein the pharmaceutical composition does not contain an antimicrobial preservative; and wherein the pharmaceutical compositions further contains a penetration enhancer, preferably TUDCA.

In another embodiment, pharmaceutical compositions comprising S-ketamine and water are provided, wherein the pharmaceutical composition does not contain an antimicrobial preservative; and wherein the pharmaceutical compositions further contains tauroursodeoxycholic acid (TUDCA); wherein the TUDCA is present in a concentration in the range of from about 1.0 mg/mL to about 25.0 mg/mL, or any amount or range therein, preferably in a concentration in the range of from about 2.5 mg/mL to about 15 mg/mL, or any amount or range therein, preferably in a concentration in the range of from about 5 mg/mL to about 10 mg/mL, or any amount or range therein. In another embodiment, pharmaceutical compositions are provided, wherein the TUDCA is present at a concentration of about 5 mg/mL. In another embodiment, pharmaceutical compositions are provided wherein the TUDCA is present at a concentration of about 10 mg/mL.

The pharmaceutical compositions for use herein may further contain one or more additional excipients for example, wetting agents, surfactant components, solubilizing agents, thickening agents, colorant agents, antioxidant components, and the like.

Examples of a suitable antioxidant component, if used, include, but are not limited to one or more of the following: sulfites; ascorbic acid; ascorbates, such as sodium ascorbate, calcium ascorbate, or potassium ascorbate; ascorbyl palmitate; fumaric acid; ethylene diamine tetraacetic acid (EDTA) or its sodium or calcium salts; tocopherol; gallates, such as propyl gallate, octyl gallate, or dodecyl gallate; vitamin E; and mixtures thereof. The antioxidant component provides long term stability to the liquid compositions. Addition of the antioxidant component can help enhance and ensure the stability of the compositions and renders the compositions stable even after six months at 40° C. A suitable amount of the antioxidant component, if present, is about 0.01 wt.-% to about 3 wt.-%, preferably about 0.05 wt.-% to about 2 wt.-%, of the total weight of the composition.

Solubilizing and emulsifying agents can be included to facilitate more uniform dispersion of the active ingredient or other excipient that is not generally soluble in the liquid carrier. Examples of a suitable emulsifying agent, if used, include, but are not limited to, for example, gelatin, cholesterol, acacia, tragacanth, pectin, methyl cellulose, carbomer, and mixtures thereof. Examples of a suitable solubilizing agent include polyethylene glycol, glycerin, D-mannitol, trehalose, benzyl benzoate, ethanol, trisaminomethane, cholesterol, triethanolamine, sodium carbonate, sodium citrate, sodium salicylate, sodium acetate, and mixtures thereof.

Preferably, the solubilizing agent includes glycerin. The solubilizing or emulsifying agent is/are generally present in an amount sufficient to dissolve or disperse the active ingredient, i.e. S-ketamine, in the carrier. Typical amounts when a solubilizing or an emulsifier are included are from about 1 wt.-% to about 80 wt.-%, preferably about 20 wt.-% to about 65 wt.-%, and more preferably about 25 wt.-% to about 55 wt.-%, of the total weight of the composition.

A suitable isotonizing agent, if used, includes sodium chloride, glycerin, D-mannitol, D-sorbitol, glucose, and mixtures thereof. A suitable amount of the isotonizing agent, when included, is typically about 0.01 wt.-% to about 15 wt.-%, more preferably about 0.3 wt.-% to about 4 wt.-%, and more preferably about 0.5 wt.-% to about 3 wt.-%, of the total weight of the composition.

A suspending agent or viscosity increasing agent can be added to the pharmaceutical compositions, to for example, increase the residence time in the nose. Suitably examples include, but are not limited to, hydroxypropyl methylcellulose, sodium carmellose, microcrystalline cellulose, carbomer, pectin, sodium alginate, chitosan salts, gellan gum, poloxamer, polyvinyl pyrrolidone, xanthan gum, and the like.

### Aspects

Aspect 1. A method of reducing symptoms of major depressive disorder, including suicidality, in a human patient assessed to be at imminent risk for suicide, comprising determining if the patient has previously attempted suicide, and if the patient has previously attempted suicide, treating the patient with (a) a standard of care treatment and (b) a therapeutically effective amount of esketamine.

Aspect 2. The method of Aspect 1, wherein if the patient is determined not to have attempted suicide, treating the patient with the standard of care treatment without treating the patient with esketamine.

Aspect 3. The method of Aspect 1 or 2, wherein the standard of care treatment comprises in-patient psychiatric hospitalization and initiation or optimization of standard antidepressant medication, as determined by a treating physician.

Aspect 4. The method of Aspect 3, wherein the standard of care treatment further comprises visits to an outpatient psychiatric facility after discharge from the in-patient psychiatric hospitalization.

Aspect 5. The method of any one of the preceding claims, wherein the symptoms comprise suicidal ideation with intent to commit suicide.

Aspect 6. The method of Aspect 1 or any one of Aspects 3-5, wherein treating the patient with a therapeutically effect amount of esketamine comprises administering about 56 mg to about 84 mg of esketamine per treatment session, wherein the treatment session occurs at a frequency of twice weekly for a treatment period that has a duration of about 4 weeks.

Aspect 7. The method of Aspect 6, wherein the previous suicide attempt was within one month prior to the first treatment session.

Aspect 8. The method of Aspect 6, wherein about 84 mg of esketamine is administered per treatment session.

Aspect 9. The method of any one of the preceding Aspects, wherein the esketamine is delivered intranasally.

Aspect 10. The method of Aspect 8, wherein the esketamine is delivered from an intranasal administration device in 2 or more sprays.

### Abbreviations

ANCOVA analysis of covariance

CGI-SR-I Clinical Global Impression—Imminent Suicide Risk

CGI-SS Clinical Global Impression—Severity of Suicidality

CGI-SS-R Clinical Global Impression—Severity of Suicidality—Revised

CrI credible interval

DB double-blind

DNA deoxyribonucleic acid

DSM-5 Diagnostic and Statistical Manual of Mental Disorders (5th edition)

ECG electrocardiogram

ECT electroconvulsive therapy

EDTA ethylenediaminetetraacetic acid

ER Emergency Room

ESK/Esk esketamine

FoST Frequency of Suicidal Thinking

ICD-10 International Statistical Classification of Diseases and Related Health Problems—10th revision

ICF informed consent form

IM intramuscular

IRT item response theory

IV Intravenous

LOCF last observation carried forward

LSD lysergic acid diethylamide

MADRS Montgomery Asberg Depression Rating Scale

MADRS-SI Montgomery Asberg Depression Rating Scale—Suicidal Thoughts Item

mAMP methamphetamine

MDD major depressive disorder

MDMA 3,4-methylenedioxy-methamphetamine

MedDRA Medical Dictionary for Regulatory Activities

MINI Mini International Psychiatric Interview

PCP phencyclidine

PD pharmacodynamic(s)

PK pharmacokinetic(s)

SIBAT Suicidal Ideation and Behavior Assessment Tool

SE standard error

SOC standard of care

TEAE treatment-emergent adverse event

TRD treatment-resistant depression

w/v weight/volume

The following Examples are set forth to aid in the understanding of the invention, and are not intended and should not be construed to limit in any way the invention set forth in the claims which follow thereafter.

### EXAMPLES

### Example 1: Enrolled Patients

Screening for eligible subjects should be performed within 48 hours prior to the first administration of intranasal study drug (if possible, screening should occur within 24 hours prior to the first administration of intranasal study drug).

The inclusion and exclusion criteria for enrolling subjects in this study are described in the following.

A. Inclusion Criteria

Each potential subject must satisfy all of the following criteria to be enrolled in the study:

1. Subject must be a man or woman, 18 to 64 years of age, inclusive.

2. Subject must meet DSM-5 diagnostic criteria for MDD, without psychotic features, based upon clinical assessment and confirmed by the MINI.

3. Subjects must have current suicidal ideation with intent, confirmed by a “Yes” response to Question B3 [Think (even momentarily) about harming or of hurting or of injuring yourself: with at least some intent or awareness that you might die as a result; or think about suicide (i.e., about killing yourself)?] AND Question B10 [Intend to act on thoughts of killing yourself?] obtained from the MINI. Note: the response to B3 must refer to the present, whereas the response to B10 may reflect the past 24 hours. If the screening period is longer than 24 hours, assessment of B3 and B10 of MINI must be repeated prior to randomization to confirm eligibility.

4. In the physician's opinion, acute psychiatric hospitalization is clinically warranted due to subject's imminent risk of suicide.

5. Subject has a MADRS total score of >28 predose on Day 1.

6. As part of standard of care treatment, subject agrees to be hospitalized voluntarily for a recommended period of 5 days after randomization (may be shorter or longer if clinically warranted in the investigator's opinion) and take prescribed noninvestigational antidepressant therapies for at least the duration of the double-blind treatment phase (Day 25).

7. Subject is comfortable with self-administration of intranasal medication and able to follow instructions provided.

8. Subject must be medically stable on the basis of physical examination, medical history, vital signs, and 12-lead ECG performed at screening. If there are abnormalities, the subject may be included only if the investigator judges the abnormalities to be not clinically significant. This determination must be recorded in the subject's source documents and initialed by the investigator.

9. Subject must be medically stable on the basis of clinical laboratory tests performed by the local laboratory at screening. If the results of the serum chemistry panel, hematology, or urinalysis are outside the normal reference ranges, the subject may be included only if the investigator judges the abnormalities or deviations from normal to be not clinically significant. This determination must be recorded in the subject's source documents and initialed by the investigator.
Incidental exclusionary laboratory values (“incidental” refers to duplicate results from a separate blood sample analyzed at the central laboratory that become available after the subject has satisfied the inclusion and exclusion criteria based on the local laboratory values) will be handled on a case-by-case basis to determine if the subject should be withdrawn from the study.

10. Contraceptive use by men or women should be consistent with local regulations regarding the use of contraceptive methods for subject participating in clinical studies.

Before randomization, a woman must be either:

a. Not of childbearing potential defined as:
postmenopausal (>45 years of age with amenorrhea for at least 12 months), permanently sterilized (e.g., bilateral tubal occlusion/ligation procedures, hysterectomy, bilateral salpingectomy, bilateral oophorectomy); or otherwise be incapable of pregnancy

b. Of childbearing potential and
practicing a highly effective method of contraception (failure rate of <1% per year when used consistently and correctly)

Examples of highly effective contraceptives include
user-independent methods: implantable progestogen-only hormone contraception associated with inhibition of ovulation; intrauterine device (IUD); intrauterine hormone-releasing system (IUS); vasectomized partner; sexual abstinence (sexual abstinence is considered a highly effective method only if defined as refraining from heterosexual intercourse during the entire period of risk associated with the study drug. The reliability of sexual abstinence needs to be evaluated in relation to the duration of the study and the preferred and usual lifestyle of the subject.)
user-dependent methods: combined (estrogen- and progestogen-containing) hormonal contraception associated with inhibition of ovulation: oral, intravaginal, and transdermal; progestogen-only hormone contraception associated with inhibition of ovulation: oral and injectable

11. A woman of childbearing potential must have a negative urine pregnancy test at screening.

12. During the study (i.e., from Day 1 of the double-blind phase) and for a minimum of 1 spermatogenesis cycle (defined as approximately 90 days) after receiving the last dose of study drug, a man who is sexually active with a woman of childbearing potential
must be practicing a highly effective method of contraception with his female partner from those listed above (see examples of highly effective methods of contraception provided for female subjects).
must use a condom if his partner is pregnant.
must agree not to donate sperm.

13. Subject must be willing and able to adhere to the prohibitions and restrictions specified in this protocol.

14. Each subject must sign an informed consent form (ICF) indicating that he or she understands the purpose of and procedures required for the study and is willing to participate in the study.

15. Each subject must sign a separate informed consent form if he or she agrees to provide an optional DNA sample for research (where local regulations permit). Refusal to give consent for the optional DNA research sample does not exclude a subject from participation in the study.

B. Exclusion Criteria Any potential subject who meets any of the following criteria will be excluded from participating in the study:

1. Subject has a current DSM-5 diagnosis of bipolar (or related disorders), antisocial personality disorder, or obsessive compulsive disorder.

2. Subject currently meets DSM-5 criteria for borderline personality disorder.
Subjects not meeting full DSM-5 criteria for borderline personality disorder but exhibiting recurrent suicidal gestures, threats, or self-mutilating behaviors should also be excluded.

3. Subject has a current clinical diagnosis of autism, dementia, or intellectual disability.

4. Subject has a current or prior DSM-5 diagnosis of a psychotic disorder, or MDD with psychotic features.

5. Subject meets the DSM-5 severity criteria for moderate or severe substance or alcohol use disorder (except for nicotine or caffeine) within the 6 months before screening.
A history (lifetime) of ketamine, phencyclidine (PCP), LSD, or MDMA hallucinogen-related use disorder is exclusionary.

6. Subject has any of the following conditions:
a history or current signs and symptoms of liver or renal insufficiency
clinically significant cardiac (including unstable coronary artery disease and congestive heart failure, tachyarrhythmias and recent myocardial infarction) or vascular, pulmonary, gastrointestinal, endocrine (including uncontrolled hyperthyroidism), neurologic (including current or past history of seizures except uncomplicated childhood febrile seizures with no sequelae), hematologic, rheumatologic, or metabolic (including severe dehydration/hypovolemia) disease.

7. Subject has uncontrolled hypertension (systolic blood pressure >140 mmHg or diastolic blood pressure >90 mmHg) despite diet, exercise or a stable dose of antihypertensive treatment for at least 2 weeks at screening; or any past history of hypertensive crisis.
Subjects with conditions in which the elevation of blood pressure could be a serious risk (including unstable heart failure, severe cardiovascular disease, recent cerebral injury, increased intracranial pressure/intracranial mass lesion, intracranial bleeding or acute stroke, untreated glaucoma or perforating eye injury) are excluded.
An abnormal blood pressure value at screening can be repeated once after 5 minutes of relaxation for subject eligibility. On Day 1 of the double-blind phase prior to randomization, a supine or semi-supine systolic blood pressure >140 mmHg or diastolic blood pressure >90 mmHg is exclusionary.

8. Subject has a positive urine test result(s) for phencyclidine (PCP), cocaine, or amphetamines (inclusive of amphetamine, mAMP, and MDMA) at screening.
Subjects who have a positive test due to the appropriate use of prescribed opiates, benzodiazepines, or barbiturates may be eligible for study participation per clinician judgment. In addition, subjects who have a positive test for opiates, benzodiazepines, or barbiturates used without a prescription, may be considered eligible per clinician judgment and in consultation with the sponsor's medical monitor. Subjects known to be using heroin should be excluded from the study.
Subjects who have a positive test due to opiates, benzodiazepines, or barbiturates taken in a suicide attempt (e.g., overdose) may be eligible for study participation per clinician judgment and in consultation with the sponsor's medical monitor.

9. Subject has a history of malignancy within 5 years before screening (exceptions are squamous and basal cell carcinomas of the skin and carcinoma in situ of the cervix, or malignancy that in the opinion of the investigator, with concurrence with the sponsor's medical monitor, is considered to have minimal risk of recurrence).

10. Subject has any anatomical or medical condition that, per the investigator's clinical judgment based on assessment, may impede delivery or absorption of intranasal study drug.

11. Subject has known allergies, hypersensitivity, intolerance or contraindications to esketamine or ketamine or its excipients.

12. Subject has taken any disallowed therapies as noted in Table 1.

TABLE 1
Prohibited Therapies

| | Episodic | Contin- | |
|---|---|---|---|
| | Use | uous | |
| Drug Class | (PRN) | Use | Comments |
| ADHD | N | Y | See also “Psychostimulants” |
| medications | | | row |
| (e.g., atomoxetine, | | | |
| guanfacine) | | | |
| Amantadine | N | N | |
| Anorexiants (e.g., | N | N | |
| phenteramine) | | | |
| Anticonvulsants | N | N | Subjects with seizures are |
| | | | excluded. Anticonvulsants |
| | | | used for other indications |
| | | | may be allowed e.g., |
| | | | valproate for migraine, |
| | | | lamotrigine for mood |
| | | | disorder). Approval for use |
| | | | can be discussed on a case- |
| | | | by-case basis with the |
| | | | sponsor’s medical monitor. |
| Antidepressants | N | Y | While continuous use of |
| (except | | | tricyclic antidepressants |
| monoamine | | | (TCAs) is not prohibited, |
| oxidase inhibitors) | | | given the target population |
| | | | (patients at imminent risk for |
| | | | suicide) and the known risk |
| | | | of lethality in TCA overdose, |
| | | | caution should be used if they |
| | | | are prescribed. |
| | | | Episodic use (PRN) of |
| | | | trazodone is permitted but |
| | | | should not be used within |
| | | | 8 hours prior to the start of |
| | | | each intranasal study drug |
| | | | administration. |
| Antidepressants: | N | N | Prohibited within the past |
| Monoamine | | | 2 weeks prior to intranasal |
| oxidase Inhibitors | | | study drug administration on |
| | | | Day 1 and are not permitted |
| | | | throughout the study. |
| Antipsychotics | Y | Y | Use of antipsychotics (except |
| | (for | | clozapine) for treatment of |
| | sleep | | depression is not excluded. It |
| | only) | | would be excluded if being |
| | | | used for psychotic symptoms. |
| | | | Episodic use (PRN) of |
| | | | antipsychotics (except |
| | | | clozapine) for sleep is |
| | | | permitted but should not be |
| | | | used within 8 hours prior to |
| | | | the start of each intranasal |
| | | | study drug administration. |
| Benzodiazepines | Y | Y | Prohibited within 8 hours |
| (at dosages | | | prior to the start of each |
| equal to or less | | | intranasal study drug |
| than the equivalent | | | administration. Additionally, |
| of 6 mg/day | | | no benzodiazepines should be |
| lorazepam) | | | used within 4 hours after the |
| | | | first intranasal study |
| | | | administration on Day 1 and |
| | | | within 8 hours of Day 2 |
| | | | assessments. |
| Chloral hydrate | N | N | |
| Clonidine | Y | Y | Prohibited within 8 hours |
| | | | prior to the start of each |
| | | | intranasal study drug |
| | | | administration. |
| Corticosteroids | Y | N | Inhaled, intranasal, topical, |
| | | | and ophthalmic steroids are |
| | | | not prohibited. Intermittent |
| | | | IM/IV corticosteroids are |
| | | | permitted (chronic use |
| | | | prohibited). Episodic or |
| | | | continuous oral use can be |
| | | | discussed on a case-by-case |
| | | | basis with sponsor’s medical |
| | | | monitor. |
| Cough/Cold/ | Y | Y | Intranasally-administered |
| Allergy | | | decongestants |
| preparations | | | (vasoconstrictors) should |
| (except those | | | not be used from 1 hour |
| containing | | | prior to each intranasal |
| dextromethorphan) | | | study medication |
| | | | administration. |
| | | | Pseudoephedrine-containing |
| | | | products should not be used |
| | | | within 12 hours prior to an |
| | | | intranasal treatment session. |
| Dextromethorphan | N | N | |
| DHEA | Y | Y | |
| Diphenhydramine | Y | N | PRN use is permitted, but |
| | | | should not be used within |
| | | | 8 hours prior to the start of |
| | | | each intranasal study drug |
| | | | administration. |
| Hypnotics | Y | Y | Do not use within 8 hours |
| (Non- | | | prior to the start of each |
| benzodiazepine | | | intranasal study drug |
| only) | | | administration. |
| Ketanserin | N | N | |
| Lithium | N | Y | Patients with bipolar disorder |
| | | | (i.e., lithium use for bipolar |
| | | | disorder) are excluded. |
| | | | Lithium use for another |
| | | | indication (e.g., augmentation |
| | | | treatment for treatment- |
| | | | resistant depression) is |
| | | | permitted. |
| Methyldopa | N | N | |
| Metyrosine | N | N | |
| Opioids | Y | Y | Prescription opioid |
| | | | medication(s) can be |
| | | | continued, per clinician’s |
| | | | judgment |
| Non-vitamin K | N | N | |
| antagonist oral | | | |
| anticoagulation | | | |
| agents (e.g., | | | |
| dabigatran, | | | |
| rivaroxaban, | | | |
| apixaban) | | | |
| Psychostimulants | N | Y | The use of amphetamines |
| (e.g., | | | (including prescribed |
| amphetamines, | | | amphetamines) can be |
| methylphenidate, | | | continued but must not be |
| and modafinil, | | | taken within 12 hours prior |
| armodafinil) | | | to the intranasal treatment |
| | | | session or for 2 hours after |
| | | | the intranasal treatment |
| | | | session. |
| Reserpine | N | N | |
| Scopolamine | N | N | |
| St. John’s Wort | N | N | |
| Thyroid hormone | N | N | Subjects needing supplements |
| Supplement | | | must be on a stable thyroid |
| | | | supplement dose for at least |
| | | | 4 weeks prior to Day 1 of the |
| | | | double-blind treatment phase. |
| Warfarin | N | N | |

13. Subject has received an investigational drug (including esketamine, ketamine, or investigational vaccines) or used an invasive investigational medical device within 60 days before the planned first dose of study drug or is currently enrolled in an investigational study.

14. Subject is a woman who is pregnant, breast-feeding, or planning to become pregnant while enrolled in this study or within 3 months after the last dose of study drug.

15. Subject has any situation or condition for which, in the opinion of the investigator, participation would not be in the best interest of the subject (e.g., compromise the wellbeing) or that could prevent, limit, or confound the protocol-specified assessments.

16. Subject is an employee of the investigator or study site, with direct involvement in the proposed study or other studies under the direction of that investigator or study site, as well as family members of the employees or the investigator.

### Example 2: Dosing, Administration, and Evaluations

A. Study Medication

All intranasal doses of study medication will be self-administered under the direct supervision of the investigator or designee. Instructions for use of the device will be provided as a separate document.

On Day 1, subjects will be randomized to treatment with either intranasal esketamine 84 mg or intranasal placebo, administered two times per week for 4 weeks. Intranasal treatment sessions should not take place on consecutive days.

Food will be restricted for at least 2 hours before each administration of study medication. Drinking of any fluids will be restricted at least 30 minutes before the first nasal spray on each dosing day. If the subject has nasal congestion on the dosing day, an intranasal decongestant can be used to reduce congestion, or with the exception of the Day 1 dose, the dosing day may be delayed. If an intranasal decongestant is used to reduce congestion, it cannot be used within 1 hour prior to intranasal study drug dosing.

The first dose of study medication will be administered in the ER or other permitted setting that has appropriate staffing to manage acutely suicidal subjects. If the first dose is administered in the ER, it is recommended that the subject not be transferred from the ER to the inpatient psychiatric unit after the 4-hour postdose assessments are completed. Subjects who have been admitted directly into the inpatient psychiatric unit due to imminent risk for suicide or transferred from a medical unit (following medical stabilization for recent suicide attempt) will receive their first dose of study medication in the inpatient psychiatric unit.

Subjects will remain in the inpatient psychiatry unit for a recommended duration of 5 days, with shorter or longer hospitalizations permitted if clinically warranted per local standard of care.

Following discharge from the inpatient psychiatric unit, subsequent visits for the double-blind treatment phase will be conducted twice-weekly at an outpatient psychiatric facility through Day 25.

On all outpatient intranasal dosing days, all subjects must remain at the clinical site until study procedures have been completed and the subject is ready for discharge per clinician's assessment. The minimum time required for post-dose monitoring is 1.5 hours. Subjects should be accompanied when released from the clinical study site. Subjects must not drive a car or work with machines for 24 hours after study drug dosing. Subjects should refrain from using alcohol within 24 hours before and after each intranasal treatment session. If a subject appears intoxicated, dosing should not occur (delayed per the permitted visit window).

On each dosing day: Subjects will self-administer 1 spray into each nostril (i.e. a total of 2 sprays using 1 intranasal device) at each of the following 3 time points: t=0, 5 minutes and 10 minutes; time=0 is defined as the time of the first 100-μL spray. Subjects will use a separate intranasal device at each of these 3 time points (i.e. a total of 3 devices). Sprays to each nostril should be delivered in rapid succession at the scheduled time points. Table 2 describes how esketamine 84 mg or placebo is administered in the double-blind treatment phase.

TABLE 2
Dose Administration of Esketamine 84 mg or Placebo

| Intranasal | Time of Intranasal Device Administration | | |
|---|---|---|---|
| Treatment | 0 ^{a} | 5 minutes | 10 minutes |
| Intranasal device ^{b} | 1^{st} | 2^{nd} | 3^{rd} |
| Placebo | 1 spray of | 1 spray of | 1 spray of |
| | placebo to | placebo to | placebo to |
| | each nostril | each nostril | each nostril |
| Esketamine | 1 spray of | 1 spray of | 1 spray of |
| 84 mg | esketamine to | esketamine to | esketamine to |
| | each nostril | each nostril | each nostril |

^{a} Time 0 is defined as the time of administration of the first intranasal spray to one nostril from the first intranasal device.
^{b} One device will be used at each time point. Each individual intranasal device contains 2 sprays. The intranasal devices containing esketamine deliver 14 mg per spray, for a total of 28 mg per individual device (i.e., 2 sprays).

After the first dose (i.e. starting with the Day 4 dose or later), if required due to tolerability issues, a one-time dose reduction to intranasal esketamine 56 mg or intranasal placebo is allowed for subsequent doses. No further dose adjustment is allowed during the double-blind treatment phase. The subject would receive the decreased dose at all remaining dosing days.

Table 3 describes how esketamine 56 mg or placebo is administered in the double-blind treatment phase.

TABLE 3
Dose Administration of Esketamine 56 mg or Placebo

| Intranasal | Time of Intranasal Device Administration | | |
|---|---|---|---|
| Treatment | 0 ^{a} | 5 minutes | 10 minutes |
| Intranasal device ^{b} | 1^{st} | 2^{nd} | 3^{rd} |
| Placebo | 1 spray of placebo | 1 spray of placebo | 1 spray of placebo |
| | to each nostril | to each nostril | to each nostril |
| Esketamine 84 mg | 1 spray of esketamine | 1 spray of esketamine | 1 spray of esketamine |
| | to each nostril | to each nostril | to each nostril |

^{a} Time 0 is defined as the time of administration of the first intranasal spray to one nostril from the first intranasal device.
^{b} One device will be used at each time point. Each individual intranasal device contains 2 sprays. The intranasal devices containing esketamine deliver 14 mg per spray, for a total of 28 mg per individual device (i.e. 2 sprays).

B. Standard of Care Antidepressant Treatment

All subjects will be treated in the context of comprehensive standard clinical care, including hospitalization and the initiation or optimization of antidepressant treatment, which will be determined by the treating physician(s) based on clinical judgment and practice guidelines. The standard of care antidepressant treatment (antidepressant monotherapy or antidepressant plus augmentation therapy) will be initiated or optimized for all subjects at the time of randomization on Day 1. Subjects who are on antidepressant monotherapy from Day 1 should remain on antidepressant monotherapy through the end of double-blind phase (Day 25) whereas subjects who are on antidepressant plus augmentation therapy from Day 1 will remain on antidepressants plus augmentation therapy through the end of double-blind phase (Day 25). Eligible subjects may or may not be receiving antidepressants at the time of study entry. Dose titration/adjustments of newly initiated or optimized standard of care antidepressant treatment should occur during the first 2 weeks of double-blind treatment (i.e. by Day 15), with doses remaining stable thereafter through the end of the double-blind phase (Day 25). Subjects who are currently taking a recently initiated antidepressant treatment at screening (initiated <2 weeks prior) may continue taking the antidepressant at the current dose or at an optimized dose (dose adjustment is allowed during the first 2 weeks of double-blind treatment) through the end of the double-blind phase (Day 25), if deemed clinically appropriate by the investigator. During the double-blind treatment phase, the investigator needs to consult with the sponsor's medical monitor in advance if additional changes on antidepressant treatment are clinically indicated.

During the follow-up phase, the antidepressant treatment will be managed based on the clinician's judgment.

C. Efficacy Evaluations

(i) Montgomery-Asberg Depression Rating Scale

The primary efficacy evaluation will be the MADRS total score. The MADRS will be performed using the Structured Interview Guide for the Montgomery Asberg Depression Rating Scale. The MADRS is a clinician-rated scale designed to be used in subjects with MDD to measure depression severity and detect changes due to antidepressant treatment. The test consists of 10 items, each of which is scored from 0 (item not present or normal) to 6 (severe or continuous presence of the symptoms), for a total possible score of 60. Higher scores represent a more severe condition. The MADRS evaluates apparent sadness, reported sadness, inner tension, sleep, appetite, concentration, lassitude, interest level, pessimistic thoughts, and suicidal thoughts. The test exhibits high inter-rater reliability.

The typical recall period for the MADRS is 7 days. In this study, the MADRS will also be administered using a since last assessment recall, a 4-hour recall on Day 1 and Day 25 postdose, and a 24-hour recall on Day 2. For the MADRS performed at 4 hours postdose on Days 1 and 25, the MADRS scores for the sleep item recorded predose on the same day will be carried forward.

Whenever possible, all efforts should be made to use the same raters for the MADRS at each site to assess the same subjects throughout the study. If this is not possible, review of the appropriate prior assessments and communication with prior raters should be conducted as needed.

(ii) Suicide Ideation and Behavior Assessment Tool (SIBAT)

The SIBAT is a suicide assessment tool that captures suicidal ideation and behavior(s) as reported by patients and reviewed by clinicians permitting efficient collection and documentation of clinical impression of severity of suicidality and imminent and long-term suicide risk and treatment plans.

The SIBAT is computerized and organized into 8 modules with branching logic to allow for efficient, comprehensive, and flexible data collection from a broad base of patients who may have a wide variety of demographic, cultural and demographic backgrounds. The 8 modules of the SIBAT are divided into patient-reported (Modules 1-5) and clinician-rated (Modules 6-8) sections. This modular structure allows for customization, and the administration of specific modules can be adjusted to meet clinical needs. Responses less susceptible to change (e.g., demographics, medical history) are segregated into modules distinct from those responses more likely to fluctuate over shorter time intervals (e, current suicidal ideation). In general, the patient-reported modules document information regarding the severity of suicidal ideation and risk and protective factors associated with suicide risk and specific suicidal behaviors. Information from the patient-reported modules, plus a brief semi-structured clinician interview in Module 6, represent a comprehensive profile for assessment of the Clinical Global Impressions in Module 7, which includes the Clinical Global Impression of Severity of Suicidality-Revised (CGI-SS-R), the Clinical Global Impression of Imminent Suicide Risk (CGI-SR-I), the Clinical Global Impression of Long-Term Suicide Risk, and assessment of the frequency of suicidal thinking. An assessment of the Clinical Global Judgment of Optimal Suicide Management is included in Module 8.

The SIBAT builds on prior work used to develop scales which are available for assessing suicidality; for example, the InterSePT Scale for Suicidal Thinking (ISST), a 12-item instrument designed for the assessment of current suicidal ideation in patients with schizophrenia and schizoaffective disorders, and the Clinical Global Impression of Severity of Suicidality (CGI-SS). Module 7 (Clinical Global Impressions) of the SIBAT includes a revised version of the CGI-SS (CGI-SS-R), which are used to evaluate the key secondary objective in this study, as well as a Clinical Global Impression of Imminent Suicide Risk (CGI-SR-I), which will be used to evaluate secondary and exploratory objectives. In addition, Question 3 (patient-reported frequency of suicidal thinking) in Module 5 (My Risk) from the SIBAT will be used to evaluate the secondary objective assessing patient-reported suicidality through the end of the double-blind treatment phase.

Formal testing of inter- and intra-rater reliability and assessments of construct validity and internal consistency are conducted on the SIBAT.

(iii) Clinical Global Impression-Severity of Suicidality.

The CGI-SS-R rating is scored on a 7-point scale from 0 (normal, not at all suicidal) to 6 (among the most extremely suicidal patients) and will be based on the totality of information available to the clinician, including information from the SIBAT. The CGI-SS-R summarizes the clinician's overall impression of severity of suicidality and will be used to assess the key secondary endpoint in this study. This rating operates like numerous other CGI-severity scales that have been used in other psychiatric studies. These instruments have shown clinical validity and sensitivity to change.

TABLE 4
Considering your total clinical experience with suicidal patients and all
information now available to you, how suicidal is this patient at this time?

| Rating | Guide to Rating |
|---|---|
| 0: Normal, not at all suicidal | Not suicidal |
| 1: Questionably suicidal | Minimal ideations; little if any |
| | impulsivity for suicide, few |
| | risk factors and many protective |
| | factors; and no impact on |
| | function. |
| 2: Mildly suicidal | Occasional ideations; little if any |
| | impulsivity for suicide; few risk |
| | factors; adequate protective factors |
| | and no or minimal impact on |
| | function |
| 3: Moderately suicidal | Intermittent ideations; with possible |
| | impulsivity for suicide; may or may |
| | not have plan or recent attempt*; |
| | several risk factors; protective factors |
| | may outweigh risk factors and |
| | some impact on function. |
| 4: Markedly suicidal | Regular ideations with intent or |
| | potential for impulsive actions |
| | for suicide; may or may not have |
| | plan or recent attempt*; multiple risk |
| | factors outweigh protective factors; |
| | and marked impact on function. |
| 5: Severely suicidal | Frequent ideations with intent; well |
| | worked out suicide plan; may or may |
| | not have recent attempt*; multiple risk |
| | factors out-weigh protective factors; |
| | and major impact on function. |
| 6: Among the most extremely | Nearly constant suicidal ideations and |
| suicidal patients | intent; well worked out plan and |
| | preparations underway or recent |
| | attempt*; and severe impact on function. |

The CGI-SS-R summarizes the clinician's overall impression of severity of suicidality and will be used to assess the key secondary endpoint in this study. This rating operates like numerous other CGI-severity scales that have been used in other psychiatric studies. These instruments have shown clinical validity and sensitivity to change. The CGI-SR-I summarizes the clinician's best assessment of the likelihood that a patient will attempt suicide in the next 7 days.

(iii) Clinical Global Impression of Imminent Suicide Risk (CGI-SR-I)

The CGI-SR-I is a scale summarizing the clinician's best assessment of the likelihood that the subject will attempt suicide in the next 7 days.

The CGI-SR-I will be used to evaluate secondary objectives assessing:
Change in imminent suicide risk at 4 hours postdose on Day 1, 24 hours postdose on Day 2, and through the end of the double-blind treatment phase and exploratory objectives assessing:
Change in imminent suicide risk through the end of the follow-up phase

(iv) Mini-International Neuropsychiatric Interview (MINI)

The MINI is a short, structured diagnostic interview developed for the fifth edition of the DSM-5 and 10^{th} revision of the ICD-10 psychiatric disorders. It has an administration time of approximately 15 to 30 minutes and provides an accurate structured psychiatric interview for multicenter clinical trials. The MINI is used to confirm the diagnosis of MDD with current suicidal ideation and to determine if there are other psychiatric conditions present.

(v) Frequency of Suicidal Thinking

The FoST describes the estimate of the frequency of the participant's suicidal thinking. The FoST rating is scored on a 6-point Likert scale: 0 (never), 1 (rarely), 2 (sometimes), 3 (often), 4 (most of the time), and 5 (all of the time). CGI-FoST (Clinician rated FoST) is one of the endpoints in Module 7 of the SIBAT that the investigator chooses an answer for, based on the totality of evidence form the SIBAT. Patient Reported FoST is in Module 5 and is reported directly by the patient.

### Example 3: Study Drug

The esketamine supplied for this study is available as a clear, colorless intranasal solution of esketamine hydrochloride (16.14% weight/volume [w/v]; equivalent to 14% w/v of esketamine base) in a nasal spray pump. The solution will consist of 161.4 mg/mL esketamine hydrochloride (equivalent to 140 mg of esketamine base) formulated in 0.12 mg/mL EDTA and 1.5 mg/mL citric acid at pH of 4.5. It is provided in a nasal spray pump, which delivers 16.14 mg esketamine hydrochloride (14 mg esketamine base) per 100 μL spray. Each individual nasal spray pump (device) contains a total of 28 mg (i.e., 2 sprays).

The placebo solution will be provided as a clear, colorless intranasal solution of water for injection with a bittering agent (denatonium benzoate [Bitrex®] at a final concentration of 0.001 mg/mL) added to simulate the taste of the intranasal solution with active drug. The placebo solution will be provided in matching nasal spray pump devices. Benzalkonium chloride is added as a preservative at a concentration of 0.3 mg/mL. Each individual nasal spray pump (device) contains 2 sprays.

### Example 4: Study 1

The sample size used in this example was calculated assuming an effect size of 0.45 points in MADRS total score between esketamine and placebo, a two-sided significance level of 0.05, and a drop-out rate at 24 hours of 5%. Approximately 112 subjects were planned to be randomized to each treatment group to achieve 90% power.

A. Primary Objectives

The primary objective is to evaluate the efficacy of intranasal esketamine 84 mg compared with intranasal placebo in addition to comprehensive standard of care in reducing the symptoms of MDD, including suicidal ideation, in subjects who are assessed to be at imminent risk for suicide, as measured by the change from baseline on the MADRS total score at 24 hours post first dose.

B. Subject and Treatment Information

This is a randomized, double-blind, placebo-controlled, multicenter study to evaluate the efficacy of intranasal esketamine 84 mg compared with intranasal placebo in addition to comprehensive standard of care in reducing the symptoms of MDD, including suicidal ideation, in adult male and female patients who were assessed to be at imminent risk for suicide.

The study consisted of a screening evaluation performed within 48 hours prior to the Day 1 intranasal dose, immediately followed by a 25-day double-blind treatment phase (Day 1 to 25), and a 65-day follow-up phase (Day 26 to Day 90). The total study duration for each subject was approximately 13 weeks.

A total of 270 subjects were screened across 51 sites in 10 countries throughout the study period. Of those, 226 subjects were randomized to 1 of 2 treatment groups in a 1:1 ratio (114 in esketamine 84 mg+SOC and 112 in placebo+SOC). The randomization was stratified by study center and by the physician's assessment of the participant's need of standard of care antidepressant treatment prior to randomization on Day 1 (i.e., antidepressant monotherapy or antidepressant plus augmentation therapy).

The study consisted of a screening evaluation performed within 24 to 48 hours prior to the Day 1 dose, immediately followed by a 25-day double-blind treatment phase (Day 1 to 25) with twice-weekly dosing sessions, and a 9-week follow up phase (Day 26 to Day 90). All participants received comprehensive SOC, including in-patient psychiatric hospitalization and the initiation or optimization of standard antidepressant medication (determined by the treating physician based on clinical judgment and practice guidelines).

The first dose of study medication will be administered in the ER or other permitted setting, including the inpatient psychiatric unit. All subjects will be treated in the context of comprehensive standard clinical care, including hospitalization and the initiation or optimization of antidepressant treatment, which will be determined by the treating physician(s). The standard of care antidepressant treatment will be initiated or optimized for all subjects on Day 1.

After the first dose (i.e. starting with the Day 4 dose or later), a one-time dose reduction to intranasal esketamine 56 mg or intranasal placebo is allowed if a subject is unable to tolerate the intranasal esketamine 84 mg or placebo dose assigned at randomization. No further dose adjustment is allowed during the double-blind treatment phase.

Dose titration/adjustments of newly initiated or optimized standard of care antidepressant treatment should occur during the first 2 weeks of double-blind treatment (i.e. by Day 15), with doses remaining stable thereafter through the end of the double-blind phase (Day 25). During the follow-up phase, the antidepressant treatment will be managed based on clinician's judgment.

Subjects will remain in the inpatient psychiatry unit for a recommended duration of 5 days, with shorter or longer hospitalizations permitted if clinically warranted per local standard of care. Discharge before 5 days must be discussed and approved by the sponsor's medical monitor. Following discharge from the inpatient psychiatric unit, subsequent visits for the double-blind treatment phase will be conducted twice-weekly at an outpatient psychiatric facility through Day 25. During the follow-up phase, subjects will be monitored twice weekly for the first two weeks (Days 28, 32, 35, and 39) after study drug treatment.

C. Baseline Clinical Characteristics

The majority of participants entered into the DB phase were female, and the mean age of all participants was approximately 39 years. The mean baseline MADRS total score was over 41 (corresponding to severe depression). Of note, this mean MADRS score is even higher than that observed in the short-term studies of esketamine in treatment-resistant depression (TRD). All participants had active suicidal ideation and intent within 24 hours of randomization. At randomization, the majority (89%) of participants were rated to be moderately to extremely suicidal at baseline, as measured by the CGI-SS-R scale derived from the SIBAT. Additionally, approximately two-thirds of participants had a prior suicide attempt, with about one-third of participants having made a suicide attempt within the past month. Both the very high mean MADRS score at entry and the recent suicide attempts underscore the population's high clinical risk and the need for a rapidly acting treatment. Slightly more (55.4%) participants were treated with antidepressant monotherapy, versus antidepressant plus augmentation therapy, as the newly initiated or optimized SOC antidepressant.

TABLE 5
Number of Subjects in Each Analysis Set;
All Randomized Analysis Set

| | Placebo + SOC | Esk 84 mg + SOC | Total |
|---|---|---|---|
| All randomized | 112 | 114 | 226 |
| Safety analysis | 112 (100.0%) | 113 (99.1%) | 225 (99.6%) |
| set | | | |
| Full efficacy | 112 (100.0%) | 112 (98.2%) | 224 (99.1%) |
| analysis set | | | |
| Follow-up | 91 (81.3%) | 101 (88.6%) | 192 (85.0%) |
| analysis set | | | |

TABLE 6
Treatment Disposition; Double-blind Treatment Phase;
All Randomized Analysis Set

| | Placebo + SOC | Esk 84 mg + SOC | Total |
|---|---|---|---|
| Analysis set: All Randomized | 112 | 114 | 226 |
| Completed study treatment | 93 (83.0%) | 102 (89.5%) | 195 (86.3%) |
| Discontinued study treatment | 19 (17.0%) | 12 (10.5%) | 31 (13.7%) |
| Reason for discontinuation | | | |
| Adverse event | 5 (4.5%) | 5 (4.4%) | 10 (4.4%) |
| Withdrawal by subject | 6 (5.4%) | 2 (1.8%) | 8 (3.5%) |
| Lack of efficacy | 6 (5.4%) | 1 (0.9%) | 7 (3.1%) |
| Other | 1 (0.9%) | 3 (2.6%) | 4 (1.8%) |
| Change from voluntary to | 0 | 1 (0.9%) | l (0.4%) |
| involuntary | | | |
| hospitalization | | | |
| Lost to follow-up | 1 (0.9%) | 0 | 1 (0.4%) |

1 subject in esk 84 mg + SOC who was randomized but did not receive any study medication is included in this summary.

TABLE 7
Summary of Demographics and Baseline Characteristics; Full Efficacy Analysis Set

| | Placebo + SOC | | Esk 84 mg + SOC | | Total | |
|---|---|---|---|---|---|---|
| Analysis set: Full Efficacy | 112 | | 112 | | 224 | |
| Age, years | | | | | | |
| N | 112 | | 112 | | 224 | |
| Mean (SD) | 37.9 | (12.54) | 40.8 | (13.17) | 39.3 | (12.91) |
| Median | 38.0 | | 39.0 | | 39.0 | |
| Range | (18; 64) | | (19; 62) | | (18; 64) | |
| 18-34 | 47 | (42.0%) | 44 | (39.3%) | 91 | (40.6%) |
| 35-54 | 53 | (47.3%) | 44 | (39.3%) | 97 | (43.3%) |
| 55-64 | 12 | (10.7%) | 24 | (21.4%) | 36 | (16.1%) |
| Sex | | | | | | |
| N | 112 | | 112 | | 224 | |
| Female | 73 | (65.2%) | 65 | (58.0%) | 138 | (61.6%) |
| Male | 39 | (34.8%) | 47 | (42.0%) | 86 | (38.4%) |
| Race | | | | | | |
| N | 112 | | 112 | | 224 | |
| Asian | 28 | (25.0%) | 28 | (25.0%) | 56 | (25.0%) |
| Black or African American | 7 | (6.3%) | 4 | (3.6%) | 11 | (4.9%) |
| Native Hawaiian or other | 0 | | 1 | (0.9%) | 1 | (0.4%) |
| Pacific Islander | | | | | | |
| White | 74 | (66.1%) | 77 | (68.8%) | 151 | (67.4%) |
| Other | 2 | (1.8%) | 1 | (0.9%) | 3 | (1.3%) |
| Multiple | 1 | (0.9%) | 1 | (0.9%) | 2 | (0.9%) |
| Ethnicity | | | | | | |
| N | 111 | | 112 | | 223 | |
| Hispanic or Latino | 7 | (6.3%) | 10 | (8.9%) | 17 | (7.6%) |
| Not Hispanic or Latino | 104 | (93.7%) | 102 | (91.1%) | 206 | (92.4%) |
| Body mass index, kg/m² | | | | | | |
| N | 111 | | 112 | | 223 | |
| Mean (SD) | 26.4 | (7.13) | 26.7 | (6.28) | 26.5 | (6.70) |
| Median | 24.8 | | 25.8 | | 25.1 | |
| Range | (17; 61) | | (18; 56) | | (17; 61) | |
| Underweight <18.5 | 4 | (3.6%) | 1 | (0.9%) | 5 | (2.2%) |
| Normal 18.5-<25 | 55 | (49.5%) | 49 | (43.8%) | 104 | (46.6%) |
| Overweight 25-<30 | 27 | (24.3%) | 34 | (30.4%) | 61 | (27.4%) |
| Obese ≥30 | 25 | (22.5%) | 28 | (25.0%) | 53 | (23.8%) |
| Country | | | | | | |
| N | 112 | | 112 | | 224 | |
| South Africa | 8 | (7.1%) | 7 | (6.3%) | 15 | (6.7%) |
| Bulgaria | 10 | (8.9%) | 9 | (8.0%) | 19 | (8.5%) |
| Estonia | 1 | (0.9%) | 3 | (2.7%) | 4 | (1.8%) |
| Germany | 10 | (8.9%) | 8 | (7.1%) | 18 | (8.0%) |
| Hungary | 8 | (7.1%) | 7 | (6.3%) | 15 | (6.7%) |
| Republic of Korea | 10 | (8.9%) | 10 | (8.9%) | 20 | (8.9%) |
| Malaysia | 9 | (8.0%) | 7 | (6.3%) | 16 | (7.1%) |
| Spain | 20 | (17.9%) | 24 | (21.4%) | 44 | (19.6%) |
| Taiwan | 8 | (7.1%) | 9 | (8.0%) | 17 | (7.6%) |
| United States | 28 | (25.0%) | 28 | (25.0%) | 56 | (25.0%) |
| Standard of Care | | | | | | |
| Antidepressant Treatment | | | | | | |
| as Randomized | | | | | | |
| N | 112 | | 112 | | 224 | |
| Antidepressant | 65 | (58.0%) | 59 | (52.7%) | 124 | (55.4%) |
| Monotherapy | | | | | | |
| Antidepressant Plus | 47 | (42.0%) | 53 | (47.3%) | 100 | (44.6%) |

Augmentation Therapy

TABLE 8
Baseline Psychiatric History; Full Efficacy Analysis Set

| | Placebo + SOC | | Esk 84 mg + SOC | | Total | |
|---|---|---|---|---|---|---|
| Analysis Set: Full Efficacy | 112 | | 121 | | 224 | |
| MADRS Total Score | | | | | | |
| N | 112 | | 111 | | 223 | |
| Mean (SD) | 41.0 | (6.29) | 41.3 | (5.87) | 41.1 | (6.07) |
| Median | 42.0 | | 41.0 | | 42.0 | |
| Range | (29; 58) | | (29; 56) | | (29; 58) | |
| MADRS Item 10-Suicidal | | | | | | |
| Thoughts | | | | | | |
| N | 112 | | 111 | | 223 | |
| 0 | 0 | | 0 | | 0 | |
| 1 | 0 | | 0 | | 0 | |
| 2 | 1 | (0.9%) | 10 | | 1 | (0.4%) |
| 3 | 8 | (7.1%) | 4 | (3.6%) | 12 | (5.4%) |
| 4 | 27 | (24.1%) | 31 | (27.9%) | 58 | (26.0%) |
| 5 | 43 | (38.4%) | 49 | (44.1%) | 92 | (41.3%) |
| 6 | 33 | (29.5%) | 127 | (24.3%) | 60 | (26.9%) |
| CGI-SS-R | | | | | | |
| N | 112 | | 111 | | 223 | |
| Normal, not at all suicidal | 0 | | 0 | | 0 | |
| Questionably suicidal | !3 | (2.7%) | 5 | (4.5%) | 8 | (3.6%) |
| Mildly suicidal | 11 | (9.8%) | 6 | (5.4%) | 17 | (7.6%) |
| Moderately suicidal | 28 | (25.0%) | 29 | (26.1%) | 57 | (25.6%) |
| Markedly suicidal | 42 | (37.5%) | 38 | (34.2%) | 80 | (35.9%) |
| Severely suicidal | 27 | (24.1%) | 29 | (26.1%) | 56 | (25.1%) |
| Among the most extremely | 1 | (0.9%) | 4 | (3.6%) | 5 | (2.2%) |
| suicidal patients | | | | | | |
| MINI: Think about suicide | | | | | | |
| N | 112 | | 112 | | 224 | |
| Yes | 112 | (100.0%) | 112 | (100.0%) | 224 | (100.0%) |
| No | 0 | | 0 | | 0 | |
| MINI: Think about suicide- | | | | | | |
| Frequency | | | | | | |
| N | 112 | | 112 | | 224 | |
| Occasionally | 14 | (12.5%) | 17 | (15.2%) | 31 | (13.8%) |
| Often | 54 | (48.2%) | 49 | (43.8%) | 103 | (46.0%) |
| Very Often | 44 | (39.3%) | 46 | (41.1%) | 90 | (40.2%) |
| MINI: Think about suicide- | | | | | | |
| Intensity | | | | | | |
| N | 112 | | 112 | | 224 | |
| Mild | 8 | (7.1%) | 8 | (7.1%) | 16 | (7.1%) |
| Moderate | 43 | (38.4%) | 41 | (36.6%) | 84 | (37.5%) |
| Severe | 61 | (54.5%) | 63 | (56.3%) | 124 | (55.4%) |
| MINI: Intend to act on | | | | | | |
| thoughts of killing yourself | | | | | | |
| N | 112 | | 112 | | 224 | |
| Yes | 112 | (100.0%) | 112 | (100.0%) | 224 | (100.0%) |
| No | 0 | | 0 | | 0 | |
| SIBAT: Prior Suicide | | | | | | |
| Attempt | | | | | | |
| N | 112 | | 111 | | 223 | |
| Yes | 68 | (60.7%) | 66 | (59.5%) | 134 | (60.1%) |
| No | 44 | (39.3%) | 45 | (40.5%) | 89 | (39.9%) |
| Suicide attempt within the | | | | | | |
| last month | | | | | | |
| N | 112 | | 112 | | 224 | |
| Yes | 31 | (27.7%) | 32 | (28.6%) | 63 | (28.1%) |
| No | 81 | (72.3%) | 80 | (71.4%) | 161 | (71.9%) |

MADRS total score ranges from 0 to 60; a higher score indicates a more severe condition;
MADRS item 10-suicide score ranges from 0 to 6; a higher score indicates a more severe condition;
SIBAT Prior Suicide Attempt from Module 1: “I have made one or more attempts to end my life”

TABLE 9
Summary of Exposure to Study Agent; Double-blind
Treatment Phase; Safety Analysis Set

| | Placebo + SOC | | Esk 84 mg + SOC | |
|---|---|---|---|---|
| Analysis set: Safety | 112 | | 113 | |
| Total duration of | | | | |
| exposure (days) ^{a} | | | | |
| N | 112 | | 113 | |
| Mean (SD) | 22.5 | (6.30) | 23.3 | (5.62) |
| Median | 25.0 | | 25.0 | |
| Range | (l; 30) | | (1; 29) | |
| Total duration of | | | | |
| exposure (days) ^{a} | | | | |
| <=7 | 5 | (4.5%) | 7 | (6.2%) |
| 8-14 | 9 | (8.0%) | 0 | |
| 15-21 | 4 | (3.6%) | 3 | (2.7%) |
| 22-25 | 66 | (58.9%) | 76 | (67.3%) |
| >25 | 28 | (25.0%) | 27 | (23.9%) |
| Total number of | | | | |
| days dosed | | | | |
| 1 | 3 | (2.7%) | 5 | (4.4%) |
| 2 | 1 | (0.9%) | 0 | |
| 3 | 5 | (4.5%) | 2 | (1.8%) |
| 4 | 5 | (4.5%) | 0 | |
| 5 | 2 | (1.8%) | 1 | (0.9%) |
| 6 | 2 | (1.8%) | 2 | (1.8%) |
| 7 | 3 | (2.7%) | 5 | (4.4%) |
| 8 | 91 | (81.3%) | 98 | (86.7%) |

^{a} Total duration of exposure is defined as time between the first and the last day of study agent in the double-blind phase.

Of the 226 randomized subjects, 1 subject in esketamine 84 mg+SOC did not receive any study medication and is therefore not included in the safety and full efficacy analysis sets. In addition, 1 subject in esketamine 84 mg+SOC had a treatment-emergent adverse event of “Hallucination, visual” leading to treatment discontinuation after the first dose of study medication and did not have any post-baseline MADRS or CGI-SS-R score and is therefore included in the safety analysis set but not the full efficacy analysis set. The mean age was 39.3 years, ranging from 18 to 64 years.

Of the 226 randomized subjects, 195 (86.3%) subjects completed the 25-day double-blind treatment phase. The most frequent reason for withdrawal was adverse event, reported by 10 (4.4%) subjects. Three subjects who completed the double-blind phase did not enter the follow-up phase. Subsequently, 192 subjects entered the follow-up phase. While 5 participants in each treatment group discontinued due to adverse events more participants in the PBO+SOC group discontinued due to lack of efficacy (6 participants in the PBO+SOC group versus 1 participant in the ESK+SOC group). Likewise, more participants in the PBO+SOC group discontinued due to withdraw by subject (6 participants in the PBO+SOC group versus 2 participants in the ESK+SOC group).

Approximately 87% of participants in the ESK+SOC group received all 8 doses of study medication, whereas 81% of participants in the PBO+SOC group received all doses of study medication.

D. Results

(i) Primary Endpoint

The change in MADRS total score was significantly greater in the ESK+SOC group than in the PBO+SOC group. The mean change from baseline (SD) 2 hours post-first dose was −16.4 (11.95) for ESK+SOC and −12.8 (10.73) for PBO+SOC. The difference between treatment groups in this LOCF ANCOVA analysis was clinically meaningful and statistically significant (LS mean [SD] difference −3.8 [1.39]; two-sided p=0.006). The effect size was 0.34 at the primary time point of 24 hours.

These positive results were consistent with the sensitivity analysis using the MMRM approach. This analysis also showed that the advantage for ESK+SOC, over PBO+SOC, in the change in MADRS total score was apparent at 4 hours post-dose, as well as at the end of the DB period.

(ii) Efficacy

Statistical analysis tests were conducted at the two-sided 0.05 significance level. The multiplicity, regarding testing multiple endpoints (the primary and the key secondary), was controlled by a fixed sequence testing procedure, i.e., the key secondary hypothesis was tested only after the null hypothesis for the primary endpoint was rejected.

(iii) Primary Efficacy Endpoint:

The primary efficacy analyses are based on the full efficacy analysis set which is defined as all randomized subjects who received at least 1 dose of double-blind study medication and have both a baseline and a post-baseline evaluation for the Montgomery Xsberg Depression Rating Scale (MADRS) total score or Clinical Global Impression-Severity of Suicidality-Revised (CGI-SS-R).

MADRS consists of 10 items that cover all of the core depressive symptoms: each item is scored from 0 (symptom is not present or is normal) to 6 (severe or continuous presence of the symptom). A total score (0 to 60) is calculated by summing the scores of all 10 items. A higher score represents a more severe condition.

Based on an ANCOVA last observation carried forward (LOCF) analysis of the primary efficacy variable (change in MADRS total score from baseline to 24 hours post first dose [Day 2]), the improvement in the esketamine 84 mg+SOC group reached statistical significance (2-sided p=0.006) when compared with the placebo+SOC group. The mean (SD) change from baseline to Day 2 (LOCF) in MADRS total score was −16.4 (11.95) for esketamine 84 mg+SOC and −12.8 (10.73) for placebo+SOC, where decreases from baseline represent improvement. Based on the ANCOVA analysis, the least-square mean difference (SE) between esketamine 84 mg+SOC and placebo+SOC was −3.8 (1.39). The effect size for the primary endpoint was 0.34. An effect size of 0.3 is considered clinically meaningful for major depressive disorder.

TABLE 10
Montgomery-Asberg Depression Rating Scale (MADRS)
Total Score: Change From Baseline to 24 Hours Post
First Dose: ANCOVA LOCF Analysis; Double-blind
Treatment Phase; Full Efficacy Analysis Set

| | Placebo + SOC | | Esk 84 mg + SOC | |
|---|---|---|---|---|
| Analysis set: Full Efficacy | 112 | | 112 | |
| Baseline (DB) | | | | |
| N | 112 | | 111 | |
| Mean (SD) | 41.0 | (6.29) | 41.3 | (5.87) |
| Median | 42.0 | | 41.0 | |
| Range | (29; 58) | | (29; 56) | |
| Day 2(DB) LOCF | | | | |
| N | 112 | | 112 | |
| Mean (SD) | 28.2 | (11.97) | 24.7 | (12.12) |
| Median | 29.0 | | 26.0 | |
| Range | (1; 54) | | (0; 50) | |
| Change from baseline | | | | |
| N | 112 | | 111 | |
| Mean (SD) | −12.8 | (10.73) | −16.4 | (11.95) |

| Median | −11.0 | −15.0 | |
|---|---|---|---|
| Range | (−44; 3) | (−56; 1) | |
| 2-sided p-value | | 0.006 | |
| (minus Placebo) ^{a} | | | |
| Diff. of LS Means (SE) | | −3.8 | (1.39) |

| | |
|---|---|
| 95% CI | (−6.56; −1.09) |

^{a} Based on analysis of covariance (ANCOVA) model with treatment (placebo, esketamine 84 mg), analysis center, standard of care antidepressant treatment as randomized (antidepressant monotherapy, antidepressant plus augmentation therapy) as factors and baseline value as a covariate; MADRS total score ranges from 0 to 60; a higher score indicates a more severe condition; Negative change in score indicates improvement.

TABLE 11
Montgomery-Asberg Depression Rating Scale (MADRS) Total Score: Change From Baseline
Over Time: MMRM Observed Case; Double-blind Treatment Phase; Full Efficacy Analysis Set

| | | | | | | | | | Testing- | |
|---|---|---|---|---|---|---|---|---|---|---|
| | Reference Group | | | Testing Group | | | | | Reference | |
| | | | LS | | | LS | M.S. | Error | LS | |
| | Treatment | N | Mean | Treatment | N | Mean | Error | DF | Mean | 95% CI ^{a} |
| Day 1 (DB): | Placebo + | 112 | −10.1 | Esk 84 mg + | 110 | −12.8 | 88.5 | 190 | −2.6 | (−5.15; |
| 4 H | SOC | | | SOC | | | | | | −0.11) |
| Day 2 (DB) | Placebo + | 111 | −12.0 | Esk 84 mg + | 111 | −15.7 | 104.5 | 190 | −3.7 | (−6.44; |
| | SOC | | | SOC | | | | | | −0.98) |
| Day 4 (DB) | Placebo + | 110 | −13.7 | Esk 84 mg + | 109 | −18.3 | 110.9 | 190 | −4.6 | (−7.43; |
| | SOC | | | SOC | | | | | | −1.79) |
| Day 8 (DB) | Placebo + | 108 | −16.6 | Esk 84 mg + | 104 | −19.0 | 127.3 | 190 | −2.5 | (−5.51; |
| | SOC | | | SOC | | | | | | 0.57) |
| Day 11 (DB) | Placebo + | 103 | −18.0 | Esk 84 mg + | 100 | −20.8 | 110.8 | 190 | −2.8 | (−5.61; |
| | SOC | | | SOC | | | | | | 0.11) |
| Day 15 (DB) | Placebo + | 99 | −20.0 | Esk 84 mg + | 104 | −21.6 | 114.9 | 190 | −1.5 | (−4.47; |
| | SOC | | | SOC | | | | | | 1.39) |
| Day 18 (DB) | Placebo + | 94 | −20.8 | Esk 84 mg + | 102 | −23.1 | 112.7 | 190 | −2.3 | (−5.23; |
| | SOC | | | SOC | | | | | | 0.61) |
| Day 22 (DB) | Placebo + | 92 | −20.3 | Esk 84 mg + | 103 | −23.5 | 122.1 | 190 | −3.2 | (−6.23; |
| | SOC | | | SOC | | | | | | −0.12) |
| Day 25 (DB): | Placebo + | 92 | −21.7 | Esk 84 mg + | 96 | −24.3 | 138.5 | 190 | −2.6 | (−5.86; |
| predose | SOC | | | SOC | | | | | | 0.68) |
| Day 25 (DB): | Placebo + | 88 | −24.7 | Esk 84 mg + | 94 | −28.8 | 90.7 | 190 | −4.1 | (−6.74; |
| 4 H | SOC | | | SOC | | | | | | −1.40) |

^{a} Based on MMRM analysis with treatment (placebo, esketamine 84 mg), time, analysis center, standard of care antidepressant treatment as randomized (antidepressant monotherapy, antidepressant plus augmentation therapy), time by treatment interaction as factors and baseline value as a covariate;
MADRS total score ranges from 0 to 60; a higher score indicates a more severe condition;
Negative change in scoreindicates improvement.

(iv) Key Secondary Efficacy Endpoint

Change in CGI-SS-R from baseline to 24 hours post first dose (Day 2). The CGI-SS-R, derived from the Suicidal Ideation and Behavior Assessment Tool (SIBAT) rating is scored on a 7-point scale from 0 (normal, not at all suicidal) to 6 (among the most extremely suicidal patients).

TABLE 12
Clinical Global Impression-Severity of Suicidality-
Revised (CGI-SS-R) Score: Change From Baseline to 24
Hours Post First Dose: ANCOVA LOCF on Ranks;
Double-blind Treatment Phase; Full Efficacy Analysis Set

| | Placebo + SOC | | Esk 84 mg + SOC | |
|---|---|---|---|---|
| Analysis set: Full Efficacy | 112 | | 112 | |
| Baseline(DB) | | | | |
| N | 112 | | 111 | |
| Median (Range) | 4.0 | (1; 6) | 14.0 | (1; 6) |
| Day 2(DB) LOCF | | | | |
| N | 112 | | 112 | |
| Median (Range) | 2.5 | (0; 5) | 2.0 | (0; 6) |
| Change from baseline | | | | |
| N | 112 | | 111 | |
| Median (Range) | −1.0 | (−5; 1) | −1.0 | (−6; 2) |

| 2-sided p-value | 0.107 | |
|---|---|---|
| (minus Placebo) ^{a} | | |
| Hodges-Lehmann Est. of | 0.0 | (−1.00; 0.00) |

Treatment Diff. (95% CI)

^{a} Based on analysis of covariance (ANCOVA) model on ranks with treatment (Placebo, Esketamine 84 mg), analysis center and standard of care antidepressant treatment as randomized (antidepressant monotherapy, antidepressant plus augmentation therapy) as factors, and baseline value (unranked) as a covariate; CGI-SS-R score ranges from 0 to 6; a higher score indicates a more severe condition; Negative change in score indicates improvement.

Based on an ANCOVA LOCF analysis of the rank of change in CGI-SS-R from baseline to 24 hours post first dose, the improvement in the esketamine 84 mg+SOC group did not reach statistical significance (2-sided p=0.107) when compared with the placebo+SOC group. The median (range) change from baseline ad Day 2 (LOCF) was −1.0 (−6; 2) for esketamine 84 mg+SOC and −1.0 (−5; 1) for placebo+SOC. The Hodges-Lehmann estimate (95% CI) of the difference between esketamine 84 mg+SOC and placebo+SOC was 0.0 (−1.00; 0.00).

(v) Safety

Intranasal ESK+SOC was safe and tolerated. Overall, 100 (88.5%) subjects in the esketamine 84 mg+SOC group and 83 (74.1%) subjects in the placebo+SOC group experienced at least one TEAE during the double-blind phase. The most common (>20%) TEAEs during the double-blind phase were dizziness (35.4%), dissociation (29.2%), nausea (20.4%) for the esketamine 84 mg+SOC group, and none for the placebo+SOC group.

There was 1 death in this study. One subject in the esketamine 84 mg+SOC group had a serious AE of completed suicide during the follow-up phase, which occurred 3 days after the last dose of study medication. This event is considered not related to study medication.

Ten subjects (4 [3.5%] in esketamine 84 mg+SOC, 6 [5.4%] in placebo+SOC) experienced serious TEAEs during the double-blind treatment phase. The SAEs in the ESK+SOC group included 1 participant with each of the following events: suicide attempt, depression suicidal, worsening of depression, and diabetic ketoacidosis. The SAEs in the PBO+SOC group included 1 participant with each of the following events: suicide attempt, depression suicidal, and hypertransaminasemia; 2 participants with worsening of suicidal ideation; 1 participant with worsening of depression and aggression.

There were 10 (8.8%) and 6 (5.4%) participants with severe TEAEs in the ESK+SOC group and PBO+SOC group, respectively. Most of the events in the ESK+SOC participants (7) were considered related to study medication, versus 1 subject in the PBO+SOC group with this investigator attribution.

Twenty three subjects (13 [12.9%] in esketamine 84 mg+SOC, 10 [11.0%] in placebo+SOC) experienced serious AEs during the follow-up phase; 13 (12.9%) in the ESK+SOC group and 10 (11.0%) in the PBO+SOC group. The SAEs in the 13 participants previously randomized to ESK+SOC group included the following events: 1 completed suicide, 3 suicide attempts, 7 suicidal ideation-related events, and 3 depression-related events. The SAEs in the 10 participants previously randomized to the PBO+SOC group included the following events: 2 suicide attempts, 6 suicidal ideation-related events, and 1 depression-related event.

Study medication was permanently stopped due to an adverse event with the following rates across treatment groups: 5 (4.4%) subjects in the esketamine 84 mg+SOC group, and 5 (4.5%) subjects in the placebo+SOC group.

TABLE 13
Overall Summary of Treatment-emergent Adverse Events;
Double-blind Treatment Phase; Safety Analysis Set

| | Placebo + SOC | | Esk 84 mg + SOC | |
|---|---|---|---|---|
| Analysis set: Safety | 112 | | 113 | |
| Subjects with 1 or more: | | | | |
| TEAEs | 83 | (74.1%) | 100 | (88.5%) |
| Related TEAEs ^{a} | 47 | (42.0%) | 90 | (79.6%) |
| TEAEs leading to death ^{b} | 0 | | 0 | |
| Serious TEAEs | 6 | (5.4%) | 4 | (3.5%) |
| Related serious TEAEs | 0 | | 0 | |
| Severe TEAEs | 6 | (5.4%) | 10 | (8.8%) |
| Related severe TEAEs | 1 | (0.9%) | 7 | (6.2%) |
| TEAEs leading to | 5 | (4.5%) | 5 | (4.4%) |

discontinuation of study agent

^{a} A TEAE is categorized as related if assessed by the investigator as possibly, probably, or very likely related to study agent;
^{b} TEAEs leading to death are based on TEAE outcome of Fatal; Incidence is based on the number of subjects experiencing at least one adverse event, not the number of events.

TABLE 14
Overall Summary of Adverse Events; Follow-up Phase;
Follow-up Analysis Set

| | Placebo + SOC | | Esk 84 mg + SOC | |
|---|---|---|---|---|
| Analysis set: Follow-up | 91 | | 101 | |
| Subjects with 1 or more: | | | | |
| AEs | 39 | (42.9%) | 49 | (48.5%) |
| AEs leading to death ^{a} | 0 | | 1 | (1.0%) |
| Serious AEs | 10 | (11.0%) | 13 | (12.9%) |
| Severe AEs | 6 | (6.6%) | 6 | (5.9%) |

^{a} AEs leading to death are based on AE outcome of Fatal; Incidence is based on the number of subjects experiencing at least one adverse event, not the number of events.

TABLE 15
Number of Subjects With Treatment-emergent
Adverse Events With Frequency of at Least 5% in Any
Treatment Group by System Organ Class and Preferred
Term; Double-blind Treatment Phase; Safety Analysis Set

| | Placebo + SOC | | Esk 84 mg + SOC | |
|---|---|---|---|---|
| Analysis set: Safety | 112 | | 113 | |
| Subjects with 1 or more | 83 | (74.1%) | 100 | (88.5%) |
| TEAEs | | | | |
| System organ class | | | | |
| Preferred term | | | | |
| Nervous system disorders | 48 | (42.9%) | 77 | (68.1%) |
| Dizziness | 10 | (8.9%) | 40 | (35.4%) |
| Headache | 20 | (17.9%) | 21 | (18.6%) |
| Somnolence | 11 | (9.8%) | 21 | (18.6%) |
| Dysgeusia | 11 | (9.8%) | 16 | (14.2%) |
| Hypoaesthesia | 2 | (1.8%) | 8 | (7.1%) |
| Sedation | 2 | (1.8%) | 7 | (6.2%) |
| Dizziness postural | 2 | (1.8%) | 6 | (5.3%) |
| Psychiatric disorders | 28 | (25.0%) | 51 | (45.1%) |
| Dissociation | 4 | (3.6%) | 33 | (29.2%) |
| Insomnia | 7 | (6.3%) | 7 | (6.2%) |
| Anxiety | 9 | (8.0%) | 6 | (5.3%) |
| Gastrointestinal disorders | 30 | (26.8%) | 43 | (38.1%) |
| Nausea | 15 | (13.4%) | 23 | (20.4%) |
| Constipation | 5 | (4.5%) | 15 | (13.3%) |
| Vomiting | 7 | (6.3%) | 8 | (7.1%) |
| Investigations | 11 | (9.8%) | 28 | (24.8%) |
| Blood pressure increased | 6 | (5.4%) | 19 | (16.8%) |
| Eye disorders | 5 | (4.5%) | 13 | (11.5%) |
| Vision blurred | 5 | (4.5%) | 10 | (8.8%) |
| Ear and labyrinth disorders | 3 | (2.7%) | 9 | (8.0%) |
| Vertigo | 1 | (0.9%) | 7 | (6.2%) |

Subjects are counted only once for any given event, regardless of the number of times they actually experienced the event; Adverse events are coded using MedDRA Version 21.1.

TABLE 16
Number of Subjects With Adverse Events With Frequency
of at Least 5% in Any Treatment Group by System Organ Class
and Preferred Term; Follow-up Phase; Follow-up Analysis Set

| | Placebo + SOC | | Esk 84 mg + SOC | |
|---|---|---|---|---|
| Analysis set: Follow-up | 91 | | 101 | |
| Subjects with 1 or more AEs | 39 | (42.9%) | 49 | (48.5%) |
| System organ class | | | | |
| Preferred term | | | | |
| Psychiatric disorders | 20 | (22.0%) | 29 | (28.7%) |
| Depression | 3 | (3.3%) | 9 | (8.9%) |
| Suicidal ideation | 5 | (5.5%) | 5 | (5.0%) |
| Anxiety | 9 | (9.9%) | 3 | (3.0%) |
| Nervous system disorders | 12 | (13.2%) | 12 | (11.9%) |
| Headache | 7 | (7.7%) | 6 | (5.9%) |

Subjects are counted only once for any given event, regardless of the number of times they actually experienced the event; Adverse events are coded using MedDRA Version 21.1.

TABLE 17
Number of Subjects With Treatment-emergent Serious
Adverse Events by System Organ Class and Preferred Term;
Double-blind Treatment Phase; Safety Analysis Set

| | Placebo + SOC | | Esk 84 mg + SOC | |
|---|---|---|---|---|
| Analysis set: Safety | 112 | | 113 | |
| Subjects with 1 or more | 6 | (5.4%) | 4 | (3.5%) |
| serious TEAEs | | | | |
| System organ class | | | | |
| Preferred term | | | | |
| Psychiatric disorders | 5 | (4.5%) | 3 | (2.7%) |
| Depression suicidal | 1 | (0.9%) | 2 | (1.8%) |
| Depression | 1 | (0.9%) | 1 | (0.9%) |
| Suicide attempt | 1 | (0.9%) | 1 | (0.9%) |

| | | | |
|---|---|---|---|
| Aggression | 1 | (0.9%) | 0 |
| Suicidal ideation | 2 | (1.8%) | 0 |

| | | | |
|---|---|---|---|
| Metabolism and nutrition | 0 | 1 | (0.9%) |
| disorders | | | |
| Diabetic ketoacidosis | 0 | 1 | (0.9%) |

| | | | |
|---|---|---|---|
| Hepatobiliary disorders | 1 | (0.9%) | 0 |
| Hypertransaminasaemia | 1 | (0.9%) | 0 |

Subjects are counted only once for any given event, regardless of the number of times they actually experienced the event; Adverse events are coded using MedDRA Version 21.1.

TABLE 18
Number of Subjects With Serious Adverse
Events by System Organ Class and Preferred Term;
Follow-up Phase; Follow-up Analysis Set

| | Placebo + SOC | | Esk 84 mg + SOC | |
|---|---|---|---|---|
| Analysis set: Follow-up | 91 | | 101 | |
| Subjects with 1 or more | 10 | (11.0%) | 13 | (12.9%) |
| SAEs | | | | |
| System organ class | | | | |
| Preferred term | | | | |
| Psychiatric disorders | 9 | (9.9%) | 13 | (12.9%) |
| Depression suicidal | 3 | (3.3%) | 5 | (5.0%) |
| Suicide attempt | 2 | (2.2%) | 3 | (3.0%) |
| Suicidal ideation | 3 | (3.3%) | 2 | (2.0%) |
| Completed suicide | 0 | | 1 | (1.0%) |
| Depression | 1 | (1.1%) | 1 | (1.0%) |

| | | | |
|---|---|---|---|
| Depressive symptom | 0 | 1 | (1.0%) |
| Major depression | 0 | 1 | (1.0%) |

| Musculoskeletal | 1 | (1.1%) | 0 |
|---|---|---|---|
| and connective | | | |
| tissue disorders | | | |
| Rhabdomyolysis | 1 | (1.1%) | 0 |

Subjects are counted only once for any given event, regardless of the number of times they actually experienced the event; Adverse events are coded using MedDRA Version 21.1.

TABLE 19
Number of Subjects With Treatment-emergent Adverse Events Leading to
Discontinuation of Study Agent by System Organ Class and Preferred Term; Double-
blind Treatment Phase; Safety Analysis Set

| | Placebo + SOC | Esk 84 mg + SOC |
|---|---|---|
| Analysis set: Safety | 112 | 113 |
| Subjects with 1 or more TEAEs | 5 (4.5%) | 5 (4.4%) |
| System organ class | | |
| Preferred term | | |
| Nervous system disorders | 0 | 3 (2.7%) |
| Dizziness | 0 | 1 (0.9%) |
| Headache | 0 | 1 (0.9%) |
| Hypoaesthesia | 0 | 1 (0.9%) |
| Sedation | 0 | 1 (0.9%) |
| Somnolence | 0 | 1 (0.9%) |
| Psychiatric disorders | 2 (1.8%) | 3 (2.7%) |
| Confusional state | 0 | 1 (0.9%) |
| Dissociation | 0 | 1 (0.9%) |
| Hallucination, visual | 0 | 1 (0.9%) |
| Aggression | 1 (0.9%) | 0 |
| Suicidal ideation | 1 (0.9%) | 0 |
| Investigations | 1 (0.9%) | 1 (0.9%) |
| Blood pressure increased | 0 | 1 (0.9%) |
| Blood pressure diastolic increased | 1 (0.9%) | 0 |
| Respiratory, thoracic and mediastinal disorders | 0 | 1 (0.9%) |
| Pharyngeal hypoaesthesia | 0 | 1 (0.9%) |
| Cardiac disorders | 1 (0.9%) | 0 |
| Atrioventricular block first degree | 1 (0.9%) | 0 |
| Hepatobiliary disorders | 1 (0.9%) | 0 |
| Hypertransaminasaemia | 1 (0.9%) | 0 |

Subjects are counted only once for any given event, regardless of the number of times they actually experienced the event; Adverse events are coded using MedDRA Version 21.1.

E. Summary

In meeting its primary endpoint, this first of two Phase 3 studies of esketamine in severely ill MDD patients assessed to be at imminent risk for suicide, demonstrated ESK+SOC to be superior to PBO+SOC in rapidly and robustly reducing symptoms of depression. ESK+SOC, compared to PBO+SOC, resulted a clinically meaningful and statistically significant improvement in the participants' MADRS total score at 24 hours post-first dose. Additionally, the benefit of ESK+SOC over PBO+SOC was apparent both at 4 hours post-initial dose, as well as at the DB endpoint (Day 25 4 hours post dose).

Although participants in both treatment groups showed clinically meaningful improvement from baseline on the CGI-SS-R, the difference in these changes did not reach statistical significance at 24 hours post-first dose of study medication.

The adverse events observed in this study are consistent with the established safety profile of esketamine. The one death, due to suicide, in a female subject previously randomized to esketamine occurred 3 days after the last dose of study medication. Although she reported minimal depressive symptoms and no suicidality at her last study visit, she did have a history of 5 prior lifetime suicide attempts with the most recent occurring within a month of randomization. The depression- and suicide-related adverse events occurring in the double-blind phase were all considered unrelated to study medication, and comparable in frequency between the two treatment groups. Both subjects (1 from each treatment group) who had a suicide attempt during the double-blind phase had a prior attempt within the past month. Although depression- and suicide-related events observed in the 9-week follow-up phase occurred in slightly more subjects previously randomized to esketamine, it is important to note that more participants discontinued due to lack of efficacy in the PBO+SOC group during the DB phase. Therefore, the slight preponderance of events in the previously esketamine-treated participants during the follow-up phase may be due to the earlier discontinuation of poor responders in the PBO+SOC group. The onset of these events was dispersed over the follow-up period and showed a similar timing across the two treatment groups. Of note, all 5 participants with a suicide attempt in the follow-up period had a previous suicide attempt in the prior one month.

Psychiatric SAEs related to suicidal ideation, suicide attempt, or worsening of depression occurring during the DB phase were infrequent (<5%), occurring in 3 and 5 participants in the ESK+SOC and PBO+SOC groups, respectively. During the 9-week follow-up phase psychiatric SAEs related to suicidal ideation, suicide attempt, completed suicide, or worsening of depression occurred in 13 and 9 participants in the ESK+SOC and PBO+SOC groups, respectively.

The positive results for the primary endpoint clearly demonstrate the rapid and robust efficacy of esketamine in reducing depressive symptoms in a severely ill patient population with a lethal condition for which there is no approved treatment. Despite the very high-risk status of the participants included in this study, the rate of suicide-related adverse events was extremely low compared to expected rates based on published literature.

These results demonstrate that intranasal esketamine is an efficacious treatment for the rapid reduction of the depressive symptoms in this very ill and vulnerable patient population. The clinical benefit of esketamine, evidenced by the improvement in depressive symptoms occurring within only a few hours after first dose, provides welcome relief to patients experiencing great mental pain and suffering. Whereas standard oral antidepressants typically require several weeks before conferring any benefit, esketamine provides robust and clinically meaningful improvement within hours. Indeed, the treatment effect size observed within 24 hours of the first dose of esketamine in this study is comparable to that seen only after 4-8 weeks of oral antidepressants or augmenting agents, as well as that observed at the end of induction phase in the Phase 3 esketamine studies in TRD. Moreover, the clinical benefit of esketamine was apparent while all participants received comprehensive standard of care, consisting of in-patient psychiatric hospitalization, 4 weeks of a newly initiated or optimized oral antidepressant, and intensive psychosocial support. The appreciable advantages of esketamine throughout the dosing period are especially notable given the large nonspecific benefits afforded by the comprehensive standard of care. Participants in this study also experienced clinically important, rapid reduction in the severity of their suicidality as measured by the CGI-SS-R.

### Example 5: Study 2

This example evaluates the efficacy of intranasal esketamine compared with placebo, in addition to comprehensive standard of care (SOC), in reducing symptoms of MDD, including suicidal ideation, in patients assessed to be at imminent risk for suicide.

This is a randomized, double-blind, placebo-controlled, multicenter study to evaluate the efficacy of intranasal ESK+SOC compared with intranasal PBO+SOC, in rapidly reducing the symptoms of MDD, including suicidal ideation, in adult patients who were assessed to be at imminent risk for suicide. The primary objective is to compare the efficacy of ESK+SOC to that of PBO+SOC in reducing the symptoms of MDD, including suicidal ideation, as measured by the change from baseline on the MADRS total score at 24 hours post-first dose.

The sample size planned for this study was calculated assuming an effect size of 0.45 points in MADRS total score between esketamine and placebo, a two-sided significance level of 0.05, and a drop-out rate at 24 hours of 5%. Approximately 112 subjects were planned to be randomized to each treatment group to achieve 90% power.

A. Primary Objectives

The primary objective is to evaluate the efficacy of intranasal esketamine 84 mg compared with intranasal placebo in addition to comprehensive standard of care in reducing the symptoms of MDD, including suicidal ideation, in subjects who are assessed to be at imminent risk for suicide, as measured by the change from baseline on the MADRS total score at 24 hours post first dose.

B. Subject and Treatment Information

A total of 273 subjects were screened across 50 sites in 12 countries throughout the study period. Of those, 230 subjects were randomized to 1 of 2 treatment groups in a 1:1 ratio (115 in esketamine 84 mg+SOC and 115 in placebo+SOC). Randomization was stratified by study center and by the physician's assessment of the subject's need of standard of care antidepressant treatment (i.e., antidepressant monotherapy or an antidepressant plus augmentation therapy); the standard of care was determined prior to randomization on Day 1. All participants received comprehensive SOC, including in-patient psychiatric hospitalization and the initiation or optimization of standard antidepressant medication (determined by the treating physician based on clinical judgment and practice guidelines). Of the 230 randomized subjects, 2 subjects in placebo+SOC and 1 subject in esketamine 84 mg+SOC did not receive any study medication and are therefore not included in the safety and full efficacy analysis sets. The mean age was 40.8 years, ranging from 18 to 64 years.

The majority of participants entered into the DB phase were female. The mean baseline Montgomery-Asberg Depression Rating Scale (MADRS) total score was nearly 40 (corresponding to severe depression). All participants had active suicidal ideation and intent within 24 hours of randomization. At randomization, the majority (91%) of participants were rated to be moderately to extremely suicidal at baseline, as measured by the CGI-SS-R scale derived from the SIBAT. Additionally, over two-thirds of participants had a prior suicide attempt, with over one-quarter of participants having made a suicide attempt within the past month. Additionally, over two-thirds of participants had a prior suicide attempt, with over one-quarter of participants having made a suicide attempt within the past month. A higher proportion of participants randomized to ESK+SOC (32%) versus PBO+SOC (21%) had a recent suicide attempt. More (61%) participants were randomized to receive treatment with antidepressant plus augmentation therapy versus antidepressant monotherapy, as the newly initiated or optimized SOC antidepressant.

The study consisted of a screening evaluation performed within 48 hours prior to the Day 1 intranasal dose, immediately followed by a 25-day double-blind treatment phase (Day 1 to 25) with twice-weekly dosing sessions, and a 65-day follow-up phase (Day 26 to Day 90). The total study duration for each subject was approximately 13 weeks.

Of the 230 randomized subjects, 184 (80.0%) subjects completed the 25-day double-blind treatment phase. Approximately 75% of participants in the ESK+SOC group received all 8 doses of study medication, whereas 83% of participants in the PBO+SOC group received all doses of study medication.

More participants in the ESK+SOC discontinued due to withdraw by subject (10 participants in the ESK+SOC group versus 5 participants in the PBO+SOC). While more participants in the ESK+SOC group discontinued due to adverse events than in PBO+SOC group (9 versus 3 participants, respectively), more participants in the PBO+SOC group discontinued due to lack of efficacy than in the ESK+SOC (6 versus 2 participants, respectively). Two most frequent reasons for withdrawal were withdrawal by subject, reported by 15 (6.5%) subjects, and adverse event, reported by 12 (5.2%) subjects. One subject who completed the double-blind phase did not enter the follow-up phase. Subsequently, 183 subjects entered the follow-up phase.

TABLE 16
Number of Subjects in Each Analysis Set;
All Randomized Analysis Set

| | Placebo + SOC | Esk 84 mg + SOC | Total |
|---|---|---|---|
| All randomized | 115 | 115 | 230 |
| Safety analysis set Full efficacy analysis set | 113 (98.3%) 113 (98.3%) | 114 (99.1%) 114 (99.1%) | 227 (98.7%) 227 (98.7%) |
| Follow-up analysis set | 94 (81.7%) | 89 (77.4%) | 183 (79.6%) |

TABLE 17
Treatment Disposition; Double-blind Treatment Phase; All Randomized Analysis Set

| | Placebo + | Esk 84 mg + | |
|---|---|---|---|
| | SOC | SOC | Total |
| Analysis set: All Randomized | 115 | 115 | 230 |
| Completed study treatment Discontinued study treatment | 94 (81.7%) 21 (18.3%) | 90 (78.3%) 25 (21.7%) | 184 (80.0%) 46 (20.0%) |
| Reason for discontinuation | | | |
| Withdrawal by subject | 5 (4.3%) | 10 (8.7%) | 15 (6.5%) |
| Adverse event | 3 (2.6%) | 9 (7.8%) | 12 (5.2%) |
| Lack of efficacy | 6 (5.2%) | 2 (1.7%) | 8 (3.5%) |
| Other | 4 (3.5%) | 3 (2.6%) | 7 (3.0%) |
| Lost to follow-up | 1 (0.9%) | 1 (0.9%) | 2 (0.9%) |
| Change from voluntary to involuntary hospitalization | 1 (0.9%) | 0 | 1 (0.4%) |
| Protocol violation | 1 (0.9%) | 0 | 1 (0.4%) |

2 subjects in placebo + SOC and 1 subject in esketamine 84 mg + SOC who were randomized but did not receive any study medication is included in this summary.

TABLE 18
Summary of Demographics and Baseline Characteristics; Full Efficacy Analysis Set

| | Placebo + SOC | Esk 84 mg + SOC | Total |
|---|---|---|---|
| Analysis set: Full Efficacy | 113 | 114 | 227 |
| Age, years | | | |
| N | 113 | 114 | 227 |
| Mean (SD) | 41.4 (13.43) | 40.2 (12.73) | 40.8 (13.07) |
| Median | 44.0 | 41.0 | 42.0 |
| Range | (18; 64) | (18; 61) | (18; 64) |
| 18-34 | 40 (35.4%) | 37 (32.5%) | 77 (33.9%) |
| 35-54 | 51 (45.1%) | 59 (51.8%) | 110 (48.5%) |
| 55-64 | 22 (19.5%) | 18 (15.8%) | 40 (17.6%) |
| Sex | | | |
| N | 113 | 114 | 227 |
| Female | 67 (59.3%) | 69 (60.5%) | 136 (59.9%) |
| Male | 46 (40.7%) | 45 (39.5%) | 91 (40.1%) |
| Race | | | |
| N | 105 | 108 | 213 |
| American Indian or Alaska | 1 (1.0%) | 0 | 1 (0.5%) |
| Native | | | |
| Asian | 2 (1.9%) | 1 (0.9%) | 3 (1.4%) |
| Black or African American | 8 (7.6%) | 7 (6.5%) | 15 (7.0%) |
| Native Hawaiian or other Pacific | 1 (1.0%) | 0 | 1 (0.5%) |
| Islander | | | |
| White | 87 (82.9%) | 92 (85.2%) | 179 (84.0%) |
| Other Multiple | 6 (5.7%) 0 | 6 (5.6%) 2 (1.9%) | 12 (5.6%) 2 (0.9%) |
| Ethnicity | | | |
| N | 106 | 108 | 214 |
| Hispanic or Latino Not Hispanic or Latino | 29 (27.40) 76 (71.7%) | 28 (25.9%) 79 (73.1%) | 57 (26.6%) 155 (72.4%) |
| Unknown | 1 (0.9%) | 1 (0.9%) | 2 (0.9%) |
| Body mass index, kg/m² | | | |
| N | 111 | 114 | 225 |
| Mean (SD) | 28.3 (7.56) | 27.6 (6.40) | 27.9 (6.99) |
| Median | 26.6 | 26.9 | 26.8 |
| Range | (18; 50) | (17; 49) | (17; 50) |
| Underweight <18.5 | 2 (1.8%) | 6 (5.3%) | 8 (3.6%) |
| Normal 18.5-<25 | 42 (37.8%) | 36 (31.6%) | 78 (34.7%) |
| Overweight 25-<30 | 28 (25.2%) | 36 (31.6%) | 64 (28.4%) |
| Obese ≥30 | 39 (35.1%) | 36 (31.6%) | 75 (33.3%) |
| Country | | | |
| N | 113 | 114 | 227 |
| Argentina | 9 (8.0%) | 7 (6.1%) | 16 (7.0%) |
| Austria | 5 (4.4%) | 4 (3.5%) | 9 (4.0%) |
| Belgium | 2 (1.8%) | 1 (0.9%) | 3 (1.3%) |
| Brazil | 18 (15.9%) | 18 (15.8%) | 36 (15.9%) |
| Canada | 1 (0.9%) | 0 | 1 (0.4%) |
| Czech Republic France | 2 (1.8%) 9 (8.0%) | 1 (0.9%) 15 (13.2%) | 3 (1.3%) 24 (10.6%) |
| Lithuania | 1 (0.9%) | 2 (1.8%) | 3 (1.3%) |
| Poland | 15 (13.3%) | 18 (15.8%) | 33 (14.5%) |
| Spain Turkey | 5 (4.4%) 10 (8.8%) | 5 (4.4%) 13 (11.4%) | 10 (4.4%) 23 (10.1%) |
| United States | 36 (31.9%) | 30 (26.3%) | 66 (29.1%) |
| Standard of Care Antidepressant Treatment as Randomized | | | |
| N | 113 | 114 | 227 |
| Antidepressant Monotherapy | 43 (38.1%) | 45 (39.5%) | 88 (38.8%) |
| Antidepressant Plus Augmentation Therapy | 70 (61.9%) | 69 (60.5%) | 139 (61.2%) |
| Standard of Care Antidepressant Treatment as Actually Received | | | |
| N | 113 | 114 | 227 |
| Antidepressant Monotherapy | 36 (31.9%) | 43 (37.7%) | 79 (34.8%) |
| Antidepressant Plus Augmentation Therapy | 68 (60.2%) | 67 (58.8%) | 135 (59.5%) |
| AD Monotherapy/AD Plus Augmentation Therapy^{a} | 9 (8.0%) | 4 (3.5%) | 13 (5.7%) |

^{a}Subjects received both antidepressant monotherapy and antidepressant augmentation therapy.

TABLE 19
Baseline Psychiatric History; Full Efficacy Analysis Set

| | Placebo + SOC | Esk 84 mg + SOC | Total |
|---|---|---|---|
| Analysis set: Full Efficacy | 113 | 114 | 227 |
| MADRS Total Score | | | |
| N | 113 | 114 | 227 |
| Mean (SD) | 39.9 (5.76) | 39.5 (5.19) | 39.7 (5.48) |
| Median | 40.0 | 39.0 | 39.0 |
| Range | (29; 54) | (29; 54) | (29; 54) |
| MADRS Item 10-Suicidal Thoughts | | | |
| N | 113 | 114 | 227 |
| 0 | 0 | 0 | 0 |
| 1 | 0 | 0 | 0 |
| 2 | 1 (0.9%) | 1 (0.9%) | 2 (0.9%) |
| 3 | 6 (5.3%) | 12 (10.5%) | 18 (7.9%) |
| 4 | 25 (22.1%) | 30 (26.3%) | 55 (24.2%) |
| 5 | 56 (49.6%) | 53 (46.5%) | 109 (48.0%) |
| 6 | 25 (22.1%) | 18 (15.8%) | 43 (18.9%) |
| Duration of Current Depressive Episode, months | | | |
| N | 106 | 100 | 206 |
| Mean (SD) | 49.9 (79.36) | 45.4 (71.07) | 47.7 (75.30) |
| Median | 21.2 | 16.5 | 17.1 |
| Range | (2; 445) | (2; 341) | (2; 445) |
| CGI-SS-R | | | |
| N | 113 | 114 | 227 |
| Normal, not at all suicidal | 0 | 0\| | 0 |
| Questionably suicidal | 3 (2.7%) | 1 (0.9%) | 4 (1.8%) |
| Mildly suicidal | 6 (5.3%) | 10 (8.8%) | 16 (7.0%) |
| Moderately suicidal | 33 (29.2%) | 35 (30.7%) | 68 (30.0%) |
| Markedly suicidal | 42 (37.2%) | 48 (42.1%) | 90 (39.6%) |
| Severely suicidal | 28 (24.8%) | 17 (14.9%) | 45 (19.8%) |
| Among the most extremely suicidal patients | 1 (0.9%) | 3 (2.6%) | 4 (1.8%) |
| CGI-SR-I | | | |
| N | 113 | 114 | 227 |
| No imminent suicide risk | 0 | 3 (2.6%) | 3 (1.3%) |
| Minimal imminent suicide risk | 4 (3.5%) | 4 (3.5%) | 8 (3.5%) |
| Mild imminent suicide risk | 10 (8.8%) | 8 (7.0%) | 18 (7.9%) |
| Moderate imminent suicide risk | 33 (29.2%) | 30 (26.3%) | 63 (27.8%) |
| Marked imminent suicide risk | 43 (38.1%) | 37 (32.5%) | 80 (35.2%) |
| Severely imminent suicide risk Extreme imminent suicide risk | 21 (18.6%) 2 (1.8%) | 28 (24.6%) 4 (3.5%) | 49 (21.6%) 6 (2.6%) |
| MINI Current Status: Think about suicide | | | |
| N | 113 | 114 | 227 |
| Yes | 113 (100.0%) | 114 (100.0%) | 227 (100.0%) |
| No | 0 | 0 | 0 |
| MINI Current Status: Think about suicide-Frequency | | | |
| N | 113 | 114 | 227 |
| Occasionally | 13 (11.5%) | 13 (11.4%) | 26 (11.5%) |
| Often | 53 (46.9%) | 58 (50.9%) | 111 (48.9%) |
| Very Often | 47 (41.6%) | 43 (37.7%) | 90 (39.6%) |
| MINI Current Status: Think about suicide-Intensity | | | |
| N | 113 | 114 | 227 |
| Mild Moderate | 5 (4.4%) 56 (49.6%) | 4 (3.5%) 52 (45.6%) | 9 (4.0%) 108 (47.6%) |
| Severe | 52 (46.0%) | 58 (50.9%) | 110 (48.5%) |
| MINI Current Status: Intend to act on thoughts of killing yourself | | | |
| N | 113 | 114 | 227 |
| Yes | 113 (100.0%) | 114 (100.0%) | 227 (100.0%) |
| No | 0 | 0 | 0 |
| MINI: Lifetime Suicide Attempt | | | |
| N | 113 | 114 | 227 |
| Yes | 74 (65.5%) | 75 (65.8%) | 149 (65.6%) |
| No | 39 (34.5%) | 39 (34.2%) | 78 (34.4%) |
| MINI: Number of Episodes of Depression in Life Time | | | |
| N | 113 | 114 | 227 |
| 1 | 26 (23.0%) | 32 (28.1%) | 58 (25.6%) |
| 2-5 | 67 (59.3%) | 58 (50.9%) | 125 (55.1%) |
| 16-10 | 10 (8.8%) | 18 (15.8%) | 28 (12.3%) |
| >10 | 10 (8.8%) | 6 (5.3%) | 16 (7.0%) |
| SIBAT: Patient Reported Frequency of Suicidal Thinking: | | | |
| N | 112 | 114 | 226 |
| I have no suicidal thoughts | 7 (6.3%) | 10 (8.8%) | 17 (7.5%) |
| I have suicidal thoughts a little of the time | 15 (13.4%) | 14 (12.3%) | 29 (12.8%) |
| I have suicidal thoughts some of the time | 33 (29.5%) | 35 (30.7%) | 68 (30.1%) |
| I have suicidal thoughts most of the time | 42 (37.5%) | 42 (36.8%) | 84 (37.2%) |
| I have suicidal thoughts all of the time | 15 (13.4%) | 13 (11.4%) | 28 (12.4%) |
| SIBAT: Prior Suicide Attempt | | | |
| N | 113 | 114 | 227 |
| Yes No | 72 (63.7%) 41 (36.3%) | 78 (68.4%) 36 (31.6%) | 150 (66.1%) 77 (33.9%) |
| Suicide attempt within the last month | | | |
| N | 113 | 114 | 227 |
| Yes | 24 (21.2%) | 36 (31.6%) | 60 (26.4%) |
| No | 89 (78.8%) | 78 (68.4%) | 167 (73.6%) |

MADRS total score ranges from 0 to 60; a higher score indicates a more severe condition; MADRS item 10-suicide score ranges from 0 to 6; a higher score indicates a more severe condition; Duration of current depressive episode is derived from MADRS euthymic baseline; SIBAT Prior Suicide Attempt from Module 1: “I have made one or more attempts to end my life”; SIBAT Patient Reported Frequency of Suicidal Thinking from Module 5, Question 3: “Which of the following ratings best describes your thinking about suicide right now?”

TABLE 20
Summary of Exposure to Study Agent; Double-blind Treatment Phase; Safety Analysis Set

| | Placebo + SOC | Esk 84 mg + SOC |
|---|---|---|
| Analysis set: Safety | 113 | 114 |
| Total duration of exposure (days) ^{a} | | |
| N | 113 | 114 |
| Mean (SD) | 22.5 (6.57) | 21.1 (8.36) |
| Median | 25.0 | 25.0 |
| Range | (1; 32) | (1; 34) |
| Total duration of exposure (days) ^{a} | | |
| <=7 | 7 (6.2%) | 16 (14.0%) |
| 8-14 | 7 (6.2%) | 7 (6.1%) |
| 15-21 | 5 (4.4%) | 1 (0.9%) |
| 22-25 | 61 (54.0%) | 56 (49.1%) |
| >25 | 33 (29.2%) | 34 (29.8%) |
| Total number of days dosed | | |
| N | 113 | 114 |
| Mean (SD) | 7.2 (1.85) | 6.8 (2.36) |
| Median | 8.0 | 8.0 |
| Range | (1; 8) | (1; 8) |
| Total number of days dosed | | |
| 1 | 3 (2.7%) | 8 (7.0%) |
| 2 | 3 (2.7%) | 7 (6.1%) |
| 3 | 4 (3.5%) | 3 (2.6%) |
| 4 | 5 (4.4%) | 3 (2.6%) |
| 5 | 0 | 3 (2.6%) |
| 6 | 4 (3.5%) | 0 |
| 7 | 0 | 4 (3.5%) |
| 8 | 94 (83.2%) | 86 (75.4%) |

^{a} Total duration of exposure is defined as time between the first and the last day of study agent in the double-blind phase.

C. Results

(i) Efficacy

Statistical analysis tests were conducted at the two-sided 0.05 significance level. The multiplicity, regarding testing multiple endpoints (the primary and the key secondary), was controlled by a fixed sequence testing procedure, i.e., the key secondary hypothesis was tested only after the null hypothesis for the primary endpoint was rejected.

(ii) Primary Efficacy Endpoint

The primary efficacy analyses are based on the full efficacy analysis set which is defined as all randomized subjects who received at least 1 dose of double-blind study medication and have both a baseline and a post-baseline evaluation for the Montgomery Asberg Depression Rating Scale (MADRS) total score or Clinical Global Impression-Severity of Suicidality-Revised (CGI-SS-R).

Primary efficacy variable/Primary timepoint: Change in MADRS total score from baseline to 24 hours post first dose (Day 2). The MADRS consists of 10 items that cover all of the core depressive symptoms: each item is scored from 0 (symptom is not present or is normal) to 6 (severe or continuous presence of the symptom). A total score (0 to 60) is calculated by summing the scores of all 10 items. A higher score represents a more severe condition.

Based on an ANCOVA last observation carried forward (LOCF) analysis of the primary efficacy variable (change in MADRS total score from baseline to 24 hours post first dose [Day 2]), the improvement in the esketamine 84 mg+SOC group reached statistical significance (2-sided p=0.006) when compared with the placebo+SOC group. The change in MADRS total score was significantly greater in the ESK+SOC group than in the PBO+SOC group. The mean (SD) change from baseline to Day 2 (LOCF) in MADRS total score was −15.7 (11.56) for esketamine 84 mg+SOC and −12.4 (10.43) for placebo+SOC, where decreases from baseline represent improvement. The difference between treatment groups in this LOCF ANCOVA analysis was clinically meaningful and statistically significant; based on the ANCOVA analysis, the least-square mean difference (SE) between esketamine 84 mg+SOC and placebo+SOC was −3.9 (1.39). The effect size for the primary endpoint was 0.35. An effect size of 0.3 is considered clinically meaningful for major depressive disorder.

These positive results were consistent with the sensitivity analysis using the MMRM approach. This analysis also showed that the advantage for ESK+SOC, over PBO+SOC, in the change in MADRS total score was apparent at 4 hours post-dose, but not at the end of the DB period.

TABLE 21
Montgomery-Asberg Depression Rating Scale (MADRS) Total Score: Change From Baseline to 24 Hours Post First Dose: ANCOVA LOCF; Double-blind Treatment Phase; Full Efficacy Analysis Set

| | Placebo + SOC | Esk 84 mg + SOC |
|---|---|---|
| Analysis set: Full Efficacy | 113 | 114 |
| Baseline(DB) | | |
| N | 113 | 114 |
| Mean (SD) | 39.9 (5.76) | 39.5 (5.19) |
| Median | 40.0 | 39.0 |
| Range | (29; 54) | (29; 54) |
| Day 2(DB) LOCF ^{a} | | |
| N | 113 | 113 |
| Mean (SD) | 27.5 (11.13) | 23.7 (11.75) |
| Median | 28.0 | 25.0 |
| Range | (1; 51) | (0; 49) |
| Change from baseline | | |
| N | 113 | 113 |
| Mean (SD) | −12.4 (10.43) | −15.7 (11.56) |
| Median | −10.0 | −14.0 |
| Range | (−51; 9) | (−48; 3) |
| 2-sided p-value (minus Placebo) ^{b} | | 0.006 |
| Diff of LS Means (SE) | | −3.9 (1.39) |
| 95% CI | | (−6.60; −1.11) |

^{a} Day 2(DB) is 24 hours post first dose;
^{b} Based on analysis of covariance (ANCOVA) model with treatment (placebo, esketamine 84 mg), analysis center, standard of care antidepressant treatment as randomized (antidepressant monotherapy, antidepressant plus augmentation therapy) as factors and baseline value as a covariate; MADRS total score ranges from 0 to 60; a higher score indicates a more severe condition; Negative change in score indicates improvement.

TABLE 22
Montgomery-Asberg Depression Rating Scale (MADRS) Total Score: Change From Baseline
Over Time: MMRM Observed Case; Double-blind Treatment Phase; Full Efficacy Analysis Set

| | | | | | | | | | Testing- | |
|---|---|---|---|---|---|---|---|---|---|---|
| | Reference Group | | | Testing Group | | | | | Reference | |
| | | | LS | | | LS | M.S. | Error | LS | |
| | Treatment | N | Mean | Treatment | N | Mean | Error | DF | Mean | 95% CI ^{a} |
| Day 1 (DB): | Placebo + | 112 | −8.2 | Esk 84 mg + | 112 | −12.4 | 68.1 | 195 | −4.2 | (−6.38; |
| 4 H | SOC | | | SOC | | | | | | −1.94) |
| Day 2 (DB) | Placebo + | 111 | −12.4 | Esk 84 mg + | 110 | −15.9 | 102.8 | 195 | −3.5 | (−6.21; |
| | SOC | | | SOC | | | | | | −0.78) |
| Day 4 (DB) | Placebo + | 111 | −15.8 | Esk 84 mg + | 107 | −17.5 | 119.3 | 195 | −1.7 | (−4.67; |
| | SOC | | | SOC | | | | | | 1.19) |
| Day 8 (DB) | Placebo + | 105 | −17.4 | Esk 84 mg + | 99 | −20.0 | 111.7 | 195 | −2.6 | (−5.53; |
| | SOC | | | SOC | | | | | | 0.26) |
| Day 11 (DB) | Placebo + | 99 | −19.4 | Esk 84 mg + | 95 | −21.5 | 122.4 | 195 | −2.1 | (−5.12; |
| | SOC | | | SOC | | | | | | 0.99) |
| Day 15 (DB) | Placebo + | 99 | −19.8 | Esk 84 mg + | 91 | −23.0 | 134.6 | 195 | −3.1 | (−6.35; |
| | SOC | | | SOC | | | | | | 0.07) |
| Day 18 (DB) | Placebo + | 90 | −21.1 | Esk 84 mg + | 84 | −22.3 | 141.4 | 195 | −1.2 | (−4.61; |
| | SOC | | | SOC | | | | | | 2.13) |
| Day 22 (DB) | Placebo + | 94 | −21.4 | Esk 84 mg + | 90 | −24.0 | 137.1 | 195 | −2.6 | (−5.89; |
| | SOC | | | SOC | | | | | | 0.72) |
| Day 25 (DB): | Placebo + | 88 | −21.5 | Esk 84 mg + | 85 | −25.3 | 138.1 | 195 | −3.7 | (−7.09; |
| predose | SOC | | | SOC | | | | | | −0.38) |
| Day 25 (DB): | Placebo + | 87 | −25.4 | Esk 84 mg + | 83 | −28.2 | 108.6 | 195 | −2.7 | (−5.75; |
| 4H | SOC | | | SOC | | | | | | 0.28) |

^{a} Based on MMRM analysis with treatment (placebo, esketamine 84 mg), time, analysis center, standard of care antidepressant treatment as randomized (antidepressant monotherapy, antidepressant plus augmentation therapy), time by treatment interaction as factors and baseline value as a covariate;
Note:
MADRS totalscore ranges from 0 to 60; a higher score indicates a more severe condition;
Negative change in score indicates improvement.

(iii) Key Secondary Efficacy Endpoint

Key secondary efficacy variable: Change in CGI-SS-R from baseline to 24 hours post first dose (Day 2). The CGI-SS-R, derived from the Suicidal Ideation and Behavior Assessment Tool (SIBAT) rating is scored on a 7-point scale from 0 (normal, not at all suicidal) to 6 (among the most extremely suicidal patients).

Based on an ANCOVA LOCF analysis of the rank of change in CGI-SS-R from baseline to 24 hours post first dose, the improvement in the esketamine 84 mg+SOC group did not reach statistical significance (2-sided p=0.379) when compared with the placebo+SOC group. The median (range) change from baseline to Day 2 (LOCF) was −1.0 (−6; 2) for esketamine 84 mg+SOC and −1.0 (−5; 2) for placebo+SOC. The Hodges-Lehmann estimate (95% CI) of the difference between esketamine 84 mg+SOC and placebo+SOC was 0.0 (0.00; 0.00).

TABLE 23
Clinical Global Impression-Severity of Suicidality-Revised (CGI-SS-R) Score: Change From Baseline to 24 Hours Post First Dose: ANCOVA LOCF on Ranks; Double-blind Treatment Phase; Full Efficacy Analysis Set

| | Placebo + SOC | Esk 84 mg + SOC |
|---|---|---|
| Analysis set: Full Efficacy | 113 | 114 |
| Baseline(DB) | | |
| N | 113 | 114 |
| Median (Range) | 14.0 (1; 6) | 4.0 (1; 6) |
| Day 2(DB) LOCF ^{a} | | |
| N | 113 | 113 |
| Median (Range) | 3.0 (0; 6) | 2.0 (0; 5) |
| Change from baseline | | |
| N | 113 | 113 |
| Median (Range) | 1.0 (−5; 2) | −1.0 (−6; 2) |
| 2-sided p-value (minus Placebo) ^{b} | | 0.379 |
| Hodges-Lehmann Est. of Treatment Diff (95% CI) | | 0.0 (0.00; 0.00) |

^{a} Day 2(DB) is 24 hours post first dose;
^{b} Based on analysis of covariance (ANCOVA) model on ranks with treatment (placebo, esketamine 84 mg), analysis center and standard of care antidepressant treatment as randomized (antidepressant monotherapy, antidepressant plus augmentation therapy) as factors, and baseline value (unranked) as a covariate.
Note:
CGI-SS-R score ranges from 0 to 6; a higher score indicates a more severe condition. Negative change in score indicates improvement.

(iv) Safety

The adverse events observed in this study are consistent with the safety profile of esketamine seen in the previous studies. Psychiatric SAEs related to suicidal ideation, suicide attempt, or worsening of depression occurring during the DB phase were infrequent (<5%); 3 participants in each treatment group made a suicide attempt. During the 9-week follow-up phase psychiatric SAEs related to suicidal ideation, suicide attempt, or worsening of depression occurred in 9 and 6 participants in the ESK+SOC and PBO+SOC groups, respectively. More suicide attempts were observed in participants previously treated with ESK+SOC (4) than in those treated with PBO+SOC (1).

Intranasal ESK+SOC was safe and tolerated. Overall, 104 (91.2%) subjects in the esketamine 84 mg+SOC group and 87 (77.0%) subjects in the placebo+SOC group experienced at least one TEAE during the double-blind phase. The most common (>20%) TEAEs during the double-blind phase were dizziness (41.2%), dissociation (38.6%), nausea (33.3%), dysgeusia (25.4%), somnolence (22.8%), headache (21.9%), paraesthesia (20.2%) for the esketamine 84 mg+SOC group, and headache (23.0%) for the placebo+SOC group. There were no deaths during this study.

Eleven subjects (5 [4.4%] in esketamine 84 mg+SOC, 6 [5.3%] in placebo+SOC) experienced serious TEAEs during the double-blind treatment phase. Twenty one subjects (9 [10.1%] in esketamine 84 mg+SOC, 12 [12.8%] in placebo+SOC) experienced serious AEs during the follow-up phase. Most of the events in the ESK+SOC participants (17) were considered related to study medication, versus no subjects in the PBO+SOC group with this investigator attribution.

The SAEs in the ESK+SOC group included the following events: 3 suicide attempts, and 1 participant each with suicidal ideation and depersonalization/derealization disorder. The SAEs in the PBO+SOC group included the following events: 3 suicide attempts, 2 suicidal ideation-related events, and 1 participant each with depression, arrhythmia, pericardial effusion and pneumothorax.

Twenty-one participants experienced an SAE in the 9-week follow-up phase; 9 (10.1%) in the ESK+SOC group and 12 (12.8%) in the PBO+SOC group. The SAEs in the 9 participants previously randomized to ESK+SOC group included the following events: 4 suicide attempts, 3 suicidal ideation-related events, and 1 participant each with a depression-related event, acute stress disorder and hemothorax. The SAEs in the 12 participants previously randomized to the PBO+SOC group included the following events: 1 suicide attempt, 5 suicidal ideation-related events, 3 infection-related events, and 1 participant each with homicidal ideation, overdose, thyroid cancer and encephalopathy.

Study medication was permanently stopped due to an adverse event with the following rates across treatment groups: 9 (7.9%) subjects in the esketamine 84 mg+SOC group, and 3 (2.7%) subjects in the placebo+SOC group.

TABLE 24
Overall Summary of Treatment-emergent Adverse Events; Double-blind Treatment Phase; Safety Analysis Set (Study 54135419SUI3002)

| | Placebo + SOC | Esk 84 mg + SOC |
|---|---|---|
| Analysis set: Safety | 113 | 114 |
| Subjects with 1 or more: | | |
| TEAEs | 87 (77.0%) | 104 (91.2%) |
| Related TEAEs ^{a} | 47 (41.6%) | 91 (79.8%) |
| TEAEs leading to death ^{b} | 0 | 0 |
| Serious TEAEs | 6 (5.3%) | 5 (4.4%) |
| Related serious TEAEs | 0 | 2 (1.8%) |
| Severe TEAEs | 7 (6.2%) | 21 (18.4%) |
| Related severe TEAEs | 0 | 17 (14.9%) |
| TEAEs leading to discontinuation of study agent | 3 (2.7%) | 9 (7.9%) |

^{a} A TEAE is categorized as related if assessed by the investigator as possibly, probably, or very likely related to study agent;
^{b} TEAEs leading to death are based on TEAE outcome of Fatal; Incidence is based on the number of subjects experiencing at least one adverse event, not the number of events.

TABLE 25
Overall Summary of Adverse Events; Follow-up Phase; Follow-up Analysis Set

| | Placebo + SOC | Esk 84 mg + SOC |
|---|---|---|
| Analysis set: Follow-up | 94 | 89 |
| Subjects with 1 or more: | | |
| AEs | 55 (58.5%) | 53 (59.6%) |
| AEs leading to death ^{a} | 0 | 0 |
| Serious AEs | 12 (12.8%) | 9 (10.1%) |
| Severe AEs | 10 (10.6%) | 6 (6.7%) |

^{a} AEs leading to death are based on AE outcome of Fatal; Incidence is based on the number of subjects experiencing at least one adverse event, not the number of events.

TABLE 26
Number of Subjects With Treatment-emergent Adverse Events With
Frequency of at Least 5% in Any Treatment Group by System Organ Class
and Preferred Term; Double-blind Treatment Phase; Safety Analysis Set

| | Placebo + SOC | Esk 84 mg + SOC |
|---|---|---|
| Analysis set: Safety | 113 | 114 |
| Subjects with 1 or more TEAEs | 87 (77.0%) | 104 (91.2%) |
| System organ class Preferred term | | |
| Nervous system disorders Dizziness | 54 (47.8%) 21 (18.6%) | 86 (75.4%) 47 (41.2%) |
| Dysgeusia | 18 (15.9%) | 29 (25.4%) |
| Somnolence | 12 (10.6%) | 26 (22.8%) |
| Headache Paraesthesia | 26 (23.0%) 7 (6.2%) | 25 (21.9%) 23 (20.2%) |
| Sedation | 3 (2.7%) | 16 (14.0%) |
| Hypoaesthesia | 1 (0.9%) | 12 (10.5%) |
| Dizziness postural | 1 (0.9%) | 9 (7.9%) |
| Psychiatric disorders | 34 (30.1%) | 74 (64.9%) |
| Dissociation | 9 (8.0%) | 44 (38.6%) |
| Anxiety | 7 (6.2%) | 17 (14.9%) |
| Euphoric mood | 1 (0.9%) | 13 (11.4%) |
| Depersonalisation/ derealisation disorder | 0 | 9 (7.9%) |
| Insomnia | 11 (9.7%) | 9 (7.9%) |
| Suicidal ideation | 6 (5.3%) | 5 (4.4%) |
| Gastrointestinal disorders | 36 (31.9%) | 59 (51.8%) |
| Nausea | 16 (14.2%) | 38 (33.3%) |
| Vomiting | 5 (4.4%) | 18 (15.8%) |
| Paraesthesia oral | 3 (2.7%) | 14 (12.3%) |
| Dry mouth | 5 (4.4%) | 8 (7.0%) |
| Constipation | 9 (8.0%) | 17 (6.1%) |
| Hypoaesthesia oral | 2(1.8%) | 7 (6.1%) |
| Respiratory, thoracic and mediastinal disorders | 24 (21.2%) | 32 (28.1%) |
| Nasal discomfort | 9 (8.0%) | 10 (8.8%) |
| Oropharyngeal pain | 3 (2.7%) | 6 (5.3%) |
| Throat irritation | 4 (3.5%) | 6 (5.3%) |
| General disorders and administration site conditions | 11 (9.7%) | 29 (25.4%) |
| Feeling drunk | 1 (0.9%) | 6 (5.3%) |
| Eye disorders | 8 (7.1%) | 21 (18.4%) |
| Vision blurred | 6 (5.3%) | 17 (14.9%) |
| Diplopia | 0 | 6 (5.3%) |
| Skin and subcutaneous tissue disorders | 9 (8.0%) | 14 (12.3%) |
| Hyperhidrosis | 3 (2.7%) | 16 (5.3%) |
| Investigations | 10 (8.8%) | 12 (10.5%) |
| Blood pressure increased | 3 (2.7%) | 7 (6.1%) |
| Ear and labyrinth disorders | 3 (2.7%) | 11 (9.6%) |
| Vertigo | 0 | 7 (6.1%) |

Subjects are counted only once for any given event, regardless of the number of times they actually experienced the event; Adverse events are coded using MedDRA Version 21.1.

TABLE 27
Number of Subjects With Adverse Events With Frequency of at Least 5% in Any Treatment Group by System Organ Class and Preferred Term; Follow-up Phase; Follow-up Analysis Set

| | Placebo + SOC | Esk 84 mg + SOC |
|---|---|---|
| Analysis set: Follow-up | 94 | 89 |
| Subjects with 1 or more AEs | 55 (58.5%) | 53 (59.6%) |
| System organ class | | |
| Preferred term | | |
| Psychiatric disorders | 28 (29.8%) | 30 (33.7%) |
| Anxiety | 9 (9.6%) | 8 (9.0%) |
| Insomnia | 7 (7.4%) | 8 (9.0%) |
| Suicidal ideation | 7 (7.4%) | 5 (5.6%) |
| Nervous system disorders | 19 (20.2%) | 13 (14.6%) |
| Headache | 10 (10.6%) | 7 (7.9%) |
| Gastrointestinal disorders | 14 (14.9%) | 11 (12.4%) |
| Diarrhoea | 5 (5.3%) | 3 (3.4%) |

Subjects are counted only once for any given event, regardless of the number of times they actually experienced the event; Adverse events are coded using MedDRA Version 21.1.

TABLE 28
Number of Subjects With Treatment-emergent Serious Adverse Events by System Organ Class and Preferred Term; Double-blind Treatment Phase; Safety Analysis Set

| | Placebo + SOC | Esk 84 mg + SOC |
|---|---|---|
| Analysis set: Safety | 113 | 114 |
| Subjects with 1 or more serious TEAEs | 6 (5.3%) | 5 (4.4%) |
| System organ class | | |
| Preferred term | | |
| Psychiatric disorders | 4 (3.5%) | 5 (4.4%) |
| Suicide attempt | 3 (2.70) | 3 (2.6%) |
| Depersonalisation/ derealisation disorder | 0 | 1 (0.9%) |
| Suicidal ideation | 2 (1.8%) | 1 (0.9%) |
| Depression | 1 (0.9%) | 0 |
| Cardiac disorders | 2 (1.8%) | 0 |
| Arrhythmia | 1 (0.9%) | 0 |
| Pericardial effusion | 1 (0.9%) | 0 |
| Respiratory, thoracic and mediastinal disorders | 1 (0.9%) | 0 |
| Pneumothorax | 1 (0.9%) | 0 |

Subjects are counted only once for any given event, regardless of the number of times they actually experienced the event; Adverse events are coded using MedDRA Version 21.1.

TABLE 29
Number of Subjects With Serious Adverse Events by System Organ Class
and Preferred Term; Follow-up Phase; Follow-up Analysis Set

| | Placebo + SOC | Esk 84 mg + SOC |
|---|---|---|
| Analysis set: Follow-up | 94 | 189 |
| Subjects with 1 or more SAEs | 12 (12.8%) | 9 (10.1%) |
| System organ class | | |
| Preferred term | | |
| Psychiatric disorders | 6 (6.4%) | 9 (10.1%) |
| Suicide attempt | 1 (1.1%) | 4 (4.5%) |
| Suicidal ideation | 3 (3.2%) | 3 (3.4%) |
| Acute stress disorder | 0 | 1 (1.1%) |
| Major depression | 0 | 1 (1.1%) |
| Depression suicidal | 2 (2.1%) | 0 |
| Homicidal ideation | 1 (1.1%) | 0 |
| Respiratory, thoracic and mediastinal disorders | 0 | 1 (1.1%) |
| Haemothorax | 0 | 1 (1.1%) |
| Infections and infestations | 3 (3.2%) | 0 |
| Erysipelas | 1 (1.1%) | 0 |
| Pyelonephritis | 1 (1.1%) | 0 |
| Staphylococcal bacteremia | 1 (1.1%) | 0 |
| Injury, poisoning and procedural complications | 1 (1.1%) | 0 |
| Overdose | 1 (1.1%) | 0 |
| Neoplasms benign, malignant and unspecified (incl cysts and polyps) | 1 (1.1%) | 0 |
| Papillary thyroid cancer | 1 (1.1%) | 0 |
| Nervous system disorders | 1 (1.1%) | 0 |
| Encephalopathy | 1 (1.1%) | 0 |

Subjects are counted only once for any given event, regardless of the number of times they actually experienced the event; Adverse events are coded using MedDRA Version 21.1.

TABLE 30
Number of Subjects With Treatment-emergent Adverse Events Leading to Discontinuation of Study Agent by System Organ Class and Preferred Term; Double-blind Treatment Phase; Safety Analysis Set

| | Placebo + SOC | Esk 84 mg + SOC |
|---|---|---|
| Analysis set: Safety | 113 | 114 |
| Subjects with 1 or more TEAEs | 3 (2.7%) | 9 (7.9%) |
| System organ class | | |
| Preferred term | | |
| Gastrointestinal disorders | 0 | 4 (3.5%) |
| Nausea | 0 | 2 (1.8%) |
| Dyspepsia | 0 | 1 (0.9%) |
| Paraesthesia oral | 0 | 1 (0.9%) |
| Vomiting | 0 | 1 (0.9%) |
| Psychiatric disorders | 1 (0.9%) | 4 (3.5%) |
| Depersonalisation/derealisation disorder | 0 | 2 (1.8%) |
| Dissociation | 0 | 2 (1.8%) |
| Depression suicidal | 1 (0.9%) | 0 |
| Respiratory, thoracic and mediastinal disorders | 1 (0.9%) | 2 (1.8%) |
| Nasal discomfort | 0 | 1 (0.9%) |
| Throat irritation | 0 | 1 (0.9%) |
| Pneumothorax | 1 (0.9%) | 0 |
| Investigations | 0 | 1 (0.9%) |
| Blood pressure increased | 0 | 1 (0.9%) |
| Nervous system disorders | 0 | 1 (0.9%) |
| Dizziness postural | 0 | 1 (0.9%) |
| Cardiac disorders | 2 (1.8%) | 0 |
| Arrhythmia | 1 (0.9%) | 0 |
| Pericardial effusion | 1 (0.9%) | 0 |

Subjects are counted only once for any given event, regardless of the number of times they actually experienced the event; Adverse events are coded using MedDRA Version 21.1.

D. Summary

The results show that intranasal esketamine is an efficacious treatment for the rapid reduction of the depressive symptoms in this very ill and vulnerable patient population. The clinical benefit of esketamine, evidenced by the improvement in depressive symptoms occurring within only a few hours after first dose, provides welcome relief to patients experiencing great mental pain and suffering. Whereas standard oral antidepressants typically require several weeks before conferring any benefit, esketamine provides clinically meaningful improvement within hours. Indeed, the treatment effect size observed within 24 hours of the first dose of esketamine in this study is comparable to that seen only after 4-8 weeks of oral antidepressants or augmenting agents. Moreover, the clinical benefit of esketamine was apparent while all participants received comprehensive standard of care, consisting of in-patient psychiatric hospitalization, 4 weeks of a newly initiated or optimized oral antidepressant, and intensive psychosocial support. The appreciable advantages of esketamine throughout the dosing period are especially notable given the large nonspecific benefits afforded by the comprehensive standard of care. Participants in this study also experienced clinically important, rapid reduction in the severity of their suicidality as measured by the CGI-SS-R.

The adverse events observed in this study are consistent with the established safety profile of esketamine. In the double-blind phase, the frequency of SAEs potentially related to suicidality was similar between the two treatment groups, with 3 suicide attempts in each group. During the 9-week follow-up phase the frequency of SAEs potentially related to suicidality was also similar across treatment groups, though more (4) participants previously randomized to ESK+SOC had a suicide attempt than those who received PBO+SOC (1). Since prior suicide attempt is the most important predictor of subsequent attempt, the greater rate of suicide attempts in the esketamine group may be related to the higher proportion of participants with a recent suicide attempt prior to randomization. The onset of the SAEs potentially related to suicidality was dispersed over the follow-up phase in both treatment groups.

The results of this Example are consistent with those of Example 1. Both studies of esketamine in MDD patients assessed to be at imminent risk for suicide reached their Primary Endpoint, clearly demonstrating the rapid efficacy of esketamine in reducing depressive symptoms in a severely ill patient population with a lethal condition for which there is no approved treatment.

In both Examples 1 and 2, there were more suicide attempts and one completed suicide in Example 1 during the follow-up among participants previously treated with ESK+SOC versus those in the PBO+SOC group. The dispersed onset of the SAEs potentially related to suicidality over the follow-up phase, is not suggestive of an acute withdrawal effect of esketamine. Additionally, the recurrence of suicidality may be an indicator of treatment resistant depression (TRD), suggesting the need for longer term treatment with esketamine in some patients.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1-10. (canceled)

11. A method of reducing symptoms of major depressive disorder, including suicidality, in a human patient assessed to be at imminent risk for suicide, comprising determining if the patient has previously attempted suicide, and if the patient has previously attempted suicide, treating the patient with (a) a standard of care treatment and (b) a therapeutically effective amount of esketamine.

12. The method of claim 11, wherein if the patient is determined not to have attempted suicide, treating the patient with the standard of care treatment without treating the patient with esketamine.

13. The method of claim 11, wherein the standard of care treatment comprises in-patient psychiatric hospitalization and initiation or optimization of standard antidepressant medication, as determined by a treating physician.

14. The method of claim 11, wherein the standard of care treatment further comprises visits to an outpatient psychiatric facility after discharge from the in-patient psychiatric hospitalization.

15. The method of claim 11, wherein the symptoms comprise suicidal ideation with intent to commit suicide.

16. The method of claim 11, wherein treating the patient with a therapeutically effect amount of esketamine comprises administering about 56 mg to about 84 mg of esketamine per treatment session, wherein the treatment session occurs at a frequency of twice weekly for a treatment period that has a duration of about 4 weeks.

17. The method of claim 16, wherein the previous suicide attempt was within one month prior to the first treatment session.

18. The method of claim 16, wherein about 84 mg of esketamine is administered per treatment session.

19. The method of claim 11, wherein the esketamine is delivered intranasally.

20. The method of claim 19, wherein the esketamine is delivered from an intranasal administration device in 2 or more sprays.

21. The method of claim 12, wherein the standard of care treatment comprises in-patient psychiatric hospitalization and initiation or optimization of standard antidepressant medication, as determined by a treating physician.

22. The method of claim 12, wherein the standard of care treatment further comprises visits to an outpatient psychiatric facility after discharge from the in-patient psychiatric hospitalization.

23. The method of claim 12, wherein the symptoms comprise suicidal ideation with intent to commit suicide.
